(19) United States
(12) Patent Application Publication
(10) Pub. No.: US 2024/0058338 A1
(43) Pub. Date: Feb. 22, 2024

(54) COMBINATION OF RAF INHIBITOR AND MEK INHIBITOR

(71) Applicant: DAY ONE BIOPHARMACEUTICALS, INC., Brisbane, CA (US)

(72) Inventors: Samuel C. BLACKMAN, Eastsound, WA (US); Eleni VENETSANAKOS, Oakland, CA (US)

(21) Appl. No.: 18/450,638

(22) Filed: Aug. 16, 2023

Related U.S. Application Data
(63) Continuation of application No. PCT/US22/16962, filed on Feb. 18, 2022.
(60) Provisional application No. 63/151,425, filed on Feb. 19, 2021.
(60) Provisional application No. 63/173,158, filed on Apr. 9, 2021.

(51) Int. Cl. A61K 31/506; A61K 31/44; A61P 35/00

(52) U.S. Cl. CPC A61K 31/506; A61K 31/44; A61P 35/00

(57) ABSTRACT

Described herein are methods and compositions for treating subjects suffering from cancer. In some aspects, herein is described a method of treating a patient suffering from cancer comprising administering to the subject: (i) (R)-2-(1-(6-amino-5-chloropyrimidine-4-carboxamido)ethyl)-N-(5-chloro-4-(trifluoromethyppyridin-2-yl)thiazole-5-carboxamide (Compound A) or a pharmaceutically acceptable salt thereof; and (ii) a MEK inhibitor as provided herein. In some aspects, the method of treating a subject suffering from cancer comprises: identifying a subject suffering from cancer, wherein the cancer has one or more of: a RAF alteration, a RAS mutation, an NF-1 mutation, or a genomic alteration that results in a dependence on signaling through the MAPK pathway; administering to a subject: (i) (R)-2-(1-(6-amino-5-chloropyrimidine-4-carboxamido)ethyl)-N-(5-chloro-4-(trifluoromethyppyridin-2-yl)thiazole-5-carboxamide (Compound A) or a pharmaceutically acceptable salt thereof; and (ii) a MEK inhibitor as provided herein.

### CROSS-REFERENCE

This application is a Continuation Application of International Patent Application PCT/US22/16962, filed Feb. 18, 2022, which claims the benefit of U.S. Provisional Patent Application No. 63/151,425, filed on Feb. 19, 2021, and U.S. Provisional Patent Application No. 63/173,158, filed on Apr. 9, 2021, each of which is incorporated by reference herein in its entirety.

### BACKGROUND OF THE INVENTION

In 2018, there were 18.1 million new cases and 9.5 million cancer-related deaths worldwide. By 2040, the number of new cancer cases per year is expected to rise to 29.5 million and the number of cancer-related deaths to 16.4 million. In 2020, there was an estimated 1.8 million new cancer cases diagnosed and 606,520 cancer deaths in the US. Cancer remains the second most common cause of death in the US, accounting for nearly 1 of every 24 deaths. Although medical advances have improved cancer survival rates, there is still a continuing need for new and more effective treatment.

### SUMMARY OF THE INVENTION

In certain aspects, the present disclosure provides a method of treating a subject suffering from cancer, comprising administering to the subject:
(i) (R)-2-(1-(6-amino-5-chloropyrimidine-4-carboxamido)ethyl)-N-(5-chloro-4-(trifluoromethyppyridin-2-yl)thiazole-5-carboxamide (Compound A), or a pharmaceutically acceptable salt thereof; and
(ii) a MEK inhibitor or a pharmaceutically acceptable salt thereof, wherein the MEK inhibitor is pimasertib,
   wherein Compound A or a pharmaceutically acceptable salt thereof and pimasertib or a pharmaceutically acceptable salt thereof are administered in a therapeutically effective amount for treating the cancer. In some embodiments, the Compound A or a pharmaceutically acceptable salt thereof is administered to the subject in an amount of about 50 mg to about 800 mg per week or in an amount of about 100 mg/m² to about 600 mg/m² per week, and wherein the pimasertib or a pharmaceutically acceptable salt thereof is administered to the subject in an amount of about 5 mg to about 150 mg daily. In some embodiments, the Compound A or a pharmaceutically acceptable salt thereof is administered to the subject in an amount of about 200 mg to about 600 mg per week or in an amount of about 140 mg/m² to about 420 mg/m² per week, and wherein the pimasertib or a pharmaceutically acceptable salt thereof is administered to the subject in an amount of about 10 mg to about 60 mg daily. In some embodiments, the method comprising administering Compound A. In some embodiments, the method comprising administering Pimasertib HCl. In some embodiments, the subject is identified as having one or more of the following fusions: AGK:BRAF, BRAF-AGAP3, AGAP3:BRAF, TNS3:BRAF, or KIAA1549:BRAF. In some embodiments, the method comprises identifying a subject having one or more of the following fusions: AGK:BRAF, BRAF-AGAP3, AGAP3:BRAF, TNS3:BRAF, or KIAA1549:BRAF. In some embodiments, the subject is identified as having a mutation selected from: PIK3CA H1047R, KRAS G12C, KRAS G12D, and KRAS G12S. In some embodiments, the method comprises identifying a subject having a mutation selected from: KRAS G12C, KRAS G12D, and KRAS G12S. In some embodiments, the method comprises identifying a subject having a mutation selected from: KRAS G12C, KRAS G12D, and KRAS G12S. In some embodiments, the subject is identified as having a BRAF mutation selected from: BRAF G464V, BRAF Indel, BRAF L597R, BRAF G466V, BRAF G469A, BRAF K601E, and BRAF G469R. In some embodiments, the method comprises identifying a subject having a BRAF mutation selected from: BRAF G464V, BRAF Indel, BRAF L597R, BRAF G466V, BRAF G469A, BRAF K601E, and BRAF G469R. In some embodiments, the cancer is a recurrent, progressive, or refractory solid tumor with mitogen-activated protein kinase (MAPK) pathway aberration.

In another aspect, the preset disclosure provides a method of treating a subject suffering from cancer, comprising administering to the subject:
(i) (R)-2-(1-(6-amino-5-chloropyrimidine-4-carboxamido)ethyl)-N-(5-chloro-4-(trifluoromethyppyridin-2-yl)thiazole-5-carboxamide (Compound A), or a pharmaceutically acceptable salt thereof; and
(ii) a MEK inhibitor or a pharmaceutically acceptable salt thereof, wherein a total amount of the Compound A or a pharmaceutically acceptable salt thereof and the MEK inhibitor or a pharmaceutically acceptable salt thereof is therapeutically effective in treating the cancer, and
   wherein the Compound A or a pharmaceutically acceptable salt thereof is administered to the subject in an amount of about 50 mg to about 800 mg per week or in an amount of about 100 mg/m² to about 600 mg/m² per week. In some embodiments, the MAPK pathway aberration is selected from one or more BRAF mutations or fusions and KRAS mutations or fusions. In some embodiments, the BRAF mutations or fusions and KRAS mutations for fusions is selected from the following gene mutations or gene fusions: BRAF V600E, BRAF G464V, BRAF G466V, BRAF G464V, BRAF K601E, KRAS Q61, KRAS G12S, BRAF G464V, BRAF Indel, BRAF L597R, BRAF G466V, BRAF G469A, BRAF K601E, BRAF G469R, KRAS G12C, KRAS G12D, KRAS G12S, AGK:BRAF, BRAF-AGAP3, AGAP3:BRAF, TNS3:BRAF, or KIAA1549:BRAF. In some embodiments, the MEK inhibitor is selected from: cobimetinib, selumetinib, pimasertib, PD0325901, refametinib, binimetinib, BI-847325, trametinib, GDC-0623, G-573, CH5126766, CIP-137401 and a compound having a structure of

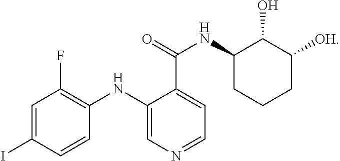 [SMILES: O=C(N[C@@H]1CCC[C@@H](O)[C@H]1O)c1ccncc1Nc1ccc(I)cc1F]

In some embodiments, the MEK inhibitor is selumetinib, binimetinib, or pimasertib. In some embodiments, the MEK inhibitor is pimasertib. In some embodiments, the cancer has one or more of the following mutations: RAS positive mutation, RAF positive mutation, MEK positive mutation, and ERK positive mutation. In some embodiments, the cancer has a RAS or RAF alteration. In some embodiments, the cancer has an NRAS mutation, a KRAS mutation, or HRAS mutation. In some embodiments, the cancer has a BRAF mutation, a BRAF fusion, or a CRAF fusion. In some embodiments, the BRAF mutation is a non-V600 BRAF mutation. In some embodiments, the BRAF mutation is a V600 BRAF mutation. In some embodiments, the cancer has a genomic alteration resulting in a dependency on signaling through the MAPK pathway. In some embodiments, the method further comprises identifying a subject suffering from cancer, wherein the cancer has one or more of: a RAF alteration, a RAS mutation, an NF-1 mutation, or a genomic alteration that results in a dependence on signaling through the MAPK pathway. In some embodiments, a cancer sample of the subject has been subjected to BRAF, KRAS, CRAF, HRAS, NF-1 and/or NRAS mutational testing prior to the administering of Compound A or a pharmaceutically acceptable salt thereof or the MEK inhibitor. In some embodiments, a cancer sample of the subject has been subjected to genomic testing prior to the administering of Compound A or a pharmaceutically acceptable salt thereof or the MEK inhibitor, wherein the genomic testing demonstrates that genomic alteration creates a dependence on MPAK signaling. In some embodiments, the patient is diagnosed with histologically confirmed non-hematologic tumor. In some embodiments, the cancer has a mutation in NF-1 resulting in NF-1 loss-of function. In some embodiments, the subject is identified having one or more of the following fusions: KIAA1549:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, AGK:BRAF, AGAP3:BRAF, TNS3:BRAF, TARDBP:BRAF, ARMC10:BRAF, CUL1:BRAF, TRIM24:BRAF, AKAP9:BRAF, FKBP15:BRAF, SKAP2:BRAF, ZKSCAN1:BRAF, KLHL7:BRAF, SEPT3:BRAF, SRGAP3:RAF1, QK1:RAF1, FYCO:RAF1, ATG7:RAF1, or NFIA:RAF1. In some embodiments, the subject is identified having one or more of the following fusions: AGK:BRAF, AGAP3:BRAF, TNS3:BRAF, or KIAA1549:BRAF. In some embodiments, the subject is identified as having AGAP3:BRAF fusion. In some embodiments, the subject is identified as having KIAA1549:BRAF fusion. In some embodiments, the non V600 BRAF mutation is selected from: V600E, G469A, G464V, G466V, K601E, G469R, and L597R. In some embodiments, the non V600 BRAF mutation is selected from: V600E, G464A, G464V, K601E, and G469R. In some embodiments, the non V600 BRAF mutation is selected from: G464V, K601E, G469A, and G466V. In some embodiments, the cancer has a KRAS mutation. In some embodiments, KRAS mutation is selected from: KRAS G12C, KRAS G12V, KRAS G12D, KRAS Q61K, KRAS Q61H, KRAS G13D, and KRAS G12S. In some embodiments, the KRAS mutation is selected from: KRAS G12C, KRAS G12D, KRAS G13D, and KRAS G12S. In some embodiments, Compound A is administered in an amount of about 100 mg to about 700 mg per week. In some embodiments, Compound A is administered at about 200 mg, about 400 mg, or 600 mg per week. In some embodiments, the subject is at least 18 years of age. In some embodiments, Compound A is administered in an amount between about 100 mg/m² to about 500 mg/m² per week. In some embodiments, Compound A is administered at about 140 mg/m², about 280 mg/m², or about 420 mg/m² per week. In some embodiments, the subject is 12, 13, 14, 15, 16, or 17 years of age. In some embodiments, Compound A is administered once weekly. In some embodiments, the MEK inhibitor or a pharmaceutically acceptable salt thereof is administered in an amount of about 10 mg to about 150 mg daily. In some embodiments, the MEK inhibitor or a pharmaceutically acceptable salt thereof is administered in an amount between about 5 mg to about 75 mg twice daily. In some embodiments, the MEK inhibitor or a pharmaceutically acceptable salt thereof is administered at about 15 mg, about 30 mg, about 45 mg, or about 60 mg twice daily. In some embodiments, the subject has not been previously administered a pan-RAF therapy. In some embodiments, the subject has not been previously administered a cytochrome P450 CYP3A4 inhibitor, a cytochrome P450 CYP2C19 inhibitor, a P450 CYP3A4 inducer, or a substrate of CYP2C9. In some embodiments, the cancer is a solid tumor. In some embodiments, the cancer is an advanced solid tumor. In some embodiments, the cancer is selected from lung cancer, colorectal cancer, pancreatic cancer, skin cancer, glioma, nonglioma brain cancer, bone sarcomas, gastrointestinal cancer, breast cancer, thyroid cancer, acute lymphocytic leukemia (ALL), acute myeloid leukemia (AML), and multiple myeloma (MM). In some embodiments, the cancer is a lung cancer, melanoma, cervix cancer, breast cancer, colorectal cancer or pancreatic cancer. In some embodiments, the cancer is a lung cancer. In some embodiments, the cancer is a recurrent or progressive solid tumor.In some embodiments, the subject has received at least one prior therapy that is considered standard of care treatment prior to the administration of Compound A or a pharmaceutically acceptable salt thereof, or the MEK inhibitor. In some embodiments, the prior therapy is a systemic therapy. In some embodiments, the prior therapy is chemotherapy therapy, hormone therapy, immunotherapy, or radiation therapy. In some embodiments, the subject has not previously received any cancer treatment. In some embodiments, a weekly dose of Compound A or a pharmaceutically acceptable salt thereof required to achieve IC80 of pERK inhibition as measured by PMA-induced peripheral blood mononuclear cell (PBMC) is at least about 5%, about 10%, about 15%, about 20%, about 30%, about 40%, or about 50% lower than the weekly dose of Compound A or a pharmaceutically acceptable salt thereof that is required in a monotherapy to achieve the same IC80 of pERK inhibition. In some embodiments, a weekly dose of the MEK inhibitor or a pharmaceutically acceptable salt thereof required to achieve IC80 of pERK inhibition as measured by PMA-induced peripheral blood mononuclear cell (PBMC) is at least about 5%, about 10%, about 15%, about 20%, about 30%, about 40%, or about 50% lower than the weekly dose of the MEK inhibitor or a pharmaceutically acceptable salt thereof that is required in a monotherapy to achieve the same IC80 of pERK inhibition.

In another aspect, the preset disclosure provides a method of treating a subject suffering from cancer, comprising administering to the subject:
(i) (R)-2-(1-(6-amino-5-chloropyrimidine-4-carboxamido)ethyl)-N-(5-chloro-4-(trifluoromethyppyridin-2-yl)thiazole-5-carboxamide (Compound A), or a pharmaceutically acceptable salt thereof; and
(ii) a MEK inhibitor or a pharmaceutically acceptable salt thereof, wherein a total amount of the Compound A or a pharmaceutically acceptable salt thereof and the MEK inhibitor or a pharmaceutically acceptable salt thereof is therapeutically effective in treating the cancer, and
   wherein the subject has one or more mitogen-activated protein kinase (MAPK) pathway aberration. In some embodiments, the MAPK pathway aberration is selected from one or more BRAF mutations or fusions and KRAS mutations or fusions. In some embodiments, the BRAF mutations or fusions and KRAS mutations for fusions is selected from the following gene mutations or gene fusions: BRAF V600E, BRAF G464V, BRAF G466V, BRAF G464V, BRAF K601E, KRAS Q61, KRAS G12S, BRAF G464V, BRAF Indel, BRAF L597R, BRAF G466V, BRAF G469A, BRAF K601E, BRAF G469R, KRAS G12C, KRAS G12D, KRAS G12S, AGK:BRAF, BRAF-AGAP3, AGAP3:BRAF, TNS3:BRAF, or KIAA1549:BRAF. In some embodiments, the MEK inhibitor is selected from: cobimetinib, selumetinib, pimasertib, PD0325901, refametinib, binimetinib, BI-847325, trametinib, GDC-0623, G-573, CH5126766, CIP-137401 and a compound having a structure of

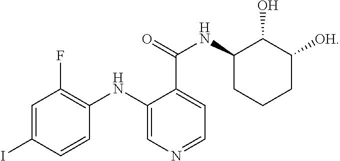 [SMILES: O=C(N[C@@H]1CCC[C@@H](O)[C@H]1O)c1ccncc1Nc1ccc(I)cc1F]

In some embodiments, the MEK inhibitor is selumetinib, binimetinib, or pimasertib. In some embodiments, the MEK inhibitor is pimasertib. In some embodiments, the cancer has one or more of the following mutations: RAS positive mutation, RAF positive mutation, MEK positive mutation, and ERK positive mutation. In some embodiments, the cancer has a RAS or RAF alteration. In some embodiments, the cancer has an NRAS mutation, a KRAS mutation, or HRAS mutation. In some embodiments, the cancer has a BRAF mutation, a BRAF fusion, or a CRAF fusion. In some embodiments, the BRAF mutation is a non-V600 BRAF mutation. In some embodiments, the BRAF mutation is a V600 BRAF mutation. In some embodiments, the cancer has a genomic alteration resulting in a dependency on signaling through the MAPK pathway. In some embodiments, the method further comprises identifying a subject suffering from cancer, wherein the cancer has one or more of: a RAF alteration, a RAS mutation, an NF-1 mutation, or a genomic alteration that results in a dependence on signaling through the MAPK pathway. In some embodiments, a cancer sample of the subject has been subjected to BRAF, KRAS, CRAF, HRAS, NF-1 and/or NRAS mutational testing prior to the administering of Compound A or a pharmaceutically acceptable salt thereof or the MEK inhibitor. In some embodiments, a cancer sample of the subject has been subjected to genomic testing prior to the administering of Compound A or a pharmaceutically acceptable salt thereof or the MEK inhibitor, wherein the genomic testing demonstrates that genomic alteration creates a dependence on MPAK signaling. In some embodiments, the patient is diagnosed with histologically confirmed non-hematologic tumor. In some embodiments, the cancer has a mutation in NF-1 resulting in NF-1 loss-of function. In some embodiments, the subject is identified having one or more of the following fusions: KIAA1549:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, AGK:BRAF, AGAP3:BRAF, TNS3:BRAF, TARDBP:BRAF, ARMC10:BRAF, CULLBRAF, TRIM24:BRAF, AKAP9:BRAF, FKBP15:BRAF, SKAP2:BRAF, ZKSCAN1:BRAF, KLHL7:BRAF, SEPT3:BRAF, SRGAP3:RAF1, QK1:RAF1, FYCO:RAF1, ATG7:RAF1, or NFIA:RAF1. In some embodiments, the subject is identified having one or more of the following fusions: AGK:BRAF, AGAP3:BRAF, TNS3:BRAF, or KIAA1549:BRAF. In some embodiments, the subject is identified as having AGAP3:BRAF fusion. In some embodiments, the subject is identified as having KIAA1549:BRAF fusion. In some embodiments, the non V600 BRAF mutation is selected from: V600E, G469A, G464V, G466V, K601E, G469R, and L597R. In some embodiments, the non V600 BRAF mutation is selected from: V600E, G464A, G464V, K601E, and G469R. In some embodiments, the non V600 BRAF mutation is selected from: G464V, K601E, G469A, and G466V. In some embodiments, the cancer has a KRAS mutation. In some embodiments, KRAS mutation is selected from: KRAS G12C, KRAS G12V, KRAS G12D, KRAS Q61K, KRAS Q61H, KRAS G13D, and KRAS G12S. In some embodiments, the KRAS mutation is selected from: KRAS G12C, KRAS G12D, KRAS G13D, and KRAS G12S. In some embodiments, Compound A is administered in an amount of about 100 mg to about 700 mg per week. In some embodiments, Compound A is administered at about 200 mg, about 400 mg, or 600 mg per week. In some embodiments, the subject is at least 18 years of age. In some embodiments, Compound A is administered in an amount between about 100 mg/m² to about 500 mg/m² per week. In some embodiments, Compound A is administered at about 140 mg/m², about 280 mg/m², or about 420 mg/m² per week. In some embodiments, the subject is 12, 13, 14, 15, 16, or 17 years of age. In some embodiments, Compound A is administered once weekly. In some embodiments, the MEK inhibitor or a pharmaceutically acceptable salt thereof is administered in an amount of about 10 mg to about 150 mg daily. In some embodiments, the MEK inhibitor or a pharmaceutically acceptable salt thereof is administered in an amount between about 5 mg to about 75 mg twice daily. In some embodiments, the MEK inhibitor or a pharmaceutically acceptable salt thereof is administered at about 15 mg, about 30 mg, about 45 mg, or about 60 mg twice daily. In some embodiments, the subject has not been previously administered a pan-RAF therapy. In some embodiments, the subject has not been previously administered a cytochrome P450 CYP3A4 inhibitor, a cytochrome P450 CYP2C19 inhibitor, a P450 CYP3A4 inducer, or a substrate of CYP2C9. In some embodiments, the cancer is a solid tumor. In some embodiments, the cancer is an advanced solid tumor. In some embodiments, the cancer is selected from lung cancer, colorectal cancer, pancreatic cancer, skin cancer, glioma, nonglioma brain cancer, bone sarcomas, gastrointestinal cancer, breast cancer, thyroid cancer, acute lymphocytic leukemia (ALL), acute myeloid leukemia (AML), and multiple myeloma (MM). In some embodiments, the cancer is a lung cancer, melanoma, cervix cancer, breast cancer, colorectal cancer or pancreatic cancer. In some embodiments, the cancer is a lung cancer. In some embodiments, the cancer is a recurrent or progressive solid tumor.In some embodiments, the subject has received at least one prior therapy that is considered standard of care treatment prior to the administration of Compound A or a pharmaceutically acceptable salt thereof, or the MEK inhibitor. In some embodiments, the prior therapy is a systemic therapy. In some embodiments, the prior therapy is chemotherapy therapy, hormone therapy, immunotherapy, or radiation therapy. In some embodiments, the subject has not previously received any cancer treatment. In some embodiments, a weekly dose of Compound A or a pharmaceutically acceptable salt thereof required to achieve IC80 of pERK inhibition as measured by PMA-induced peripheral blood mononuclear cell (PBMC) is at least about 5%, about 10%, about 15%, about 20%, about 30%, about 40%, or about 50% lower than the weekly dose of Compound A or a pharmaceutically acceptable salt thereof that is required in a monotherapy to achieve the same IC80 of pERK inhibition. In some embodiments, a weekly dose of the MEK inhibitor or a pharmaceutically acceptable salt thereof required to achieve IC80 of pERK inhibition as measured by PMA-induced peripheral blood mononuclear cell (PBMC) is at least about 5%, about 10%, about 15%, about 20%, about 30%, about 40%, or about 50% lower than the weekly dose of the MEK inhibitor or a pharmaceutically acceptable salt thereof that is required in a monotherapy to achieve the same IC80 of pERK inhibition.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates Phase 1b/2 trial design using a combination of Compound A and Pimasertib. The multi-center, open label sub-study will consist of patients ≥12 years of age, with recurrent or progressive solid tumors with aberrations in key proteins of MAPK pathway, such as tumors that harbor RAS or RAF alterations. Compound A will be administered once weekly (Days 1, 8, 15, and 22) and Pimasertib will be administered once (QD) or twice daily (BID), with cycles repeating every 28 days in the absence of disease progression or unacceptable toxicity. Abbreviations: BRAF (v-raf murine sarcoma viral oncogene homolog B); F/U (follow-up); KRAS (Kirsten rat sarcoma viral oncogene); MAPK (mitogen-activated protein kinase); and NRAS (neuroblastoma sarcoma viral oncogene).

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Aryl” refers to an aromatic mono- or polycyclic moiety with preferably 6 to 20 carbon atoms which is preferably selected from phenyl, biphenyl, naphthyl, tetrahydronaphthyl, fluorenyl, indenyl or phenanthrenyl, more preferably phenyl or naphthyl.

“Heteroaryl” refers to an aromatic moiety having 6 to 20 carbon atoms with at least one ring containing a heteroatom selected from O, N and/or S, or heteroaryl is an aromatic ring containing at least one heteroatom selected from O, N and/or S and 1 to 6 carbon atoms. Preferably, heteroaryl contains 1 to 4, more preferably 1, 2 or 3 heteroatoms selected from O and/or N and is preferably selected from pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Spiro moieties are also included within the scope of this definition. Preferred heteroaryl include pyridinyl, imidazolyl, pyrimidinyl, pyrazolyl, triazolyl, pyrazinyl, tetrazolyl, isoxazolyl, oxazolyl, isothiazolyl, oxadiazolyl, triazolyl. Heteroaryl groups are optionally mono-, di-, or trisubstituted with, e.g., halogen, lower alkyl, lower alkoxy, haloalkyl, aryl, heteroaryl, and hydroxy.

“Heterocyclyl” refers to a saturated or unsaturated ring containing at least one heteroatom selected from O, N and/or S and 1 to 6 carbon atoms. Preferably, heterocyclyl contains 1 to 4, more preferably 1, 2 or 3 heteroatoms selected from O and/or N and is preferably selected from pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, azetidin-2-one-1-yl, pyrrolidin-2-one-1-yl, piperid-2-one-1-yl, azepan-2-one-1-yl, 3-azabicyco[3.1.0]hexanyl, 3-azabicyclo[4.1.0]heptanyl, azabicyclo[2.2.2]hexanyl, 3H-indolyl and quinolizinyl. Spiromoieties are also included within the scope of this definition.

“Carbocyclyl” refers to a monocyclic or polycyclic ring system of 3 to 20 carbon atoms which may be saturated, unsaturated or aromatic.

“Alkyl” refers to a saturated hydrocarbon moiety, namely straight chain or branched alkyl having 1 to 10, preferably 1 to 8 carbon atoms, more preferably 1 to 4 carbon atoms, such as methyl, ethyl, n-propyl, isopropyl, n-butyl, isobutyl, tert-butyl, n-pentyl, isopentyl, neopentyl, hexyl or heptyl.

“Cycloalkyl” refers to an alkyl ring having 3 to 10, preferably 3 to 8 carbon atoms, more preferably 3 to 6 carbon atoms, such as cyclopropyl, cyclobutyl, cyclopentyl, cyclohexyl, cycloheptyl or cyclooctyl.

“Alkenyl” refers to an unsaturated hydrocarbon moiety with one or more double bonds, preferably one double bond, namely straight chain or branched alkenyl having 1 to 10, preferably 2 to 8 carbon atoms, more preferably 2 to 4 atoms, such as vinyl, allyl, methallyl, buten-2-yl, buten-3-yl, penten-2-yl, penten-3-yl, penten-4-yl, 3-methyl-but-3-enyl, 2-methyl-but-3-enyl, 1-methyl-but-3-enyl, hexenyl or heptenyl.

“Alkynyl” refers to an unsaturated hydrocarbon moiety with one or more triple bonds, preferably one triple bond, namely straight chain or branched alkynyl having 1 to 10, preferably 2 to 8 carbon atoms, more preferably 2 to 4 atoms, such as ethynyl, propynyl, butyn-2-yl, butyn-3-yl, pentyn-2-yl, pentyn-3-yl, pentyn-4-yl, 2-methyl-but-3-ynyl, 1-methyl-but-3-ynyl, hexynyl or heptynyl.

“Halo” or “halogen” refers to a halogen atom preferably selected from F, Cl, Br and I, preferably F, Cl and Br.

In the definitions cycloalkylalkyl, arylalkyl, heretoarylalkyl and heterocyclylalkyl it is contemplated that cycloalkyl, aryl, heretoaryl and heterocyclyl are bonded via an alkylene moiety. This alkylene moiety may be a straight chain or branched chain group. Said alkylene moiety preferably has 1 to 6 carbon atoms. Examples thereof include methylene, ethylene, n-propylene, n-butylene, n-pentylene, n-hexylene, iso-propylene, sec.-butylene, tert.-butylene, 1,1-dimethyl propylene, 1,2-dimethyl propylene, 2,2-dimethyl propylene, 1,1-dimethyl butylene, 1,2-dimethyl butylene, 1,3-dimethyl butylene, 2,2-dimethyl butylene, 2,3-dimethyl butylene, 3,3-dimethyl butylene, 1-ethyl butylene, 2-ethyl butylene, 3-ethyl butylene, 1-n-propyl propylene, 2-n-propyl propylene, 1-iso-propyl propylene, 2-iso-propyl propylene, 1-methyl pentylene, 2-methyl pentylene, 3-methyl pentylene and 4-methyl pentylene. More preferably, said alkylene moiety has 1 to 3 carbon atoms, such as methylene, ethylene, n-propylene and iso-propylene. Most preferred is methylene.

“Acyl” refers to the group —C(O)R where R includes “C₁-C₆-alkyl”, “aryl”, “heteroaryl”, “C₃-C₈-cycloalkyl”, “C₃-C₈-heterocycloalkyl”, “C₁-C₆-alkyl aryl” or “C₁-C₆-alkyl heteroaryl”. “Acyloxy” refers to the group —OC(O)R where R includes “C₁-C₆-alkyl”, “aryl”, “hetero-aryl”, “C₁-C₆-alkyl aryl” or “C₁-C₆-alkyl heteroaryl”.

“Aryl acyl” refers to aryl groups having an acyl substituent, including 2-acetylphenyl and the like.

“Heteroaryl acyl” refers to heteroaryl groups having an acyl substituent, including 2-acetylpyridyl and the like.

“Alkoxy” refers to the group —O—R where R includes “C₁-C₆-alkyl”, “C₂-C₆-alkenyl”, “C₂-C₆-alkynyl”, “C₃-C₈-cycloalkyl”, “Heterocycloalkyl”, “heterocycloalkyl”, “aryl”, “heteroaryl”, “C₂-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred alkoxy groups include by way of example, methoxy, ethoxy, phenoxy and the like.

“Alkoxycarbonyl” refers to the group C(O)OR where R includes “C₁-C₆-alkyl” or “aryl” or “heteroaryl” or “C₁-C₆-alkyl aryl” or “C₁-C₆-alkyl heteroaryl”.

“Alkoxycarbonylamino” refers to the group —NR′C(O)OR where R includes “C₁-C₆-alkyl” or “aryl” or “heteroaryl” or “C₁-C₆-alkyl aryl” or “C₁-C₆-alkyl heteroaryl” a and R′ includes hydrogen or “C₁-C₆-alkyl

“Aminocarbonyl” refers to the group C(O)NRR′ where each R, R′ includes independently hydrogen or C₁-C₆-alkyl or aryl or heteroaryl or “C₁-C₆-alkyl aryl” or “C₁-C₆-alkyl hetero-aryl”.

“Acylamino” refers to the group —NR(CO)R′ where each R. R′ is independently hydrogen or “C₁-C₆-alkyl” or “aryl” or “heteroaryl” or “C₁-C₆-alkyl aryl” or “C₁-C₆-alkyl heteroaryl”.

“Sulfonyloxy” refers to a group —OSO₂—R wherein R is selected from H, “C₁-C₆-alkyl”, “C₁-C₆-alkyl” substituted with halogens, e.g., an —OSO₂—CF₃group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Sulfinyl” refers to a group “—(O)—R” wherein R is selected from H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “Heterocycloalkyl”, “heterocycloalkyl”³, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Sulfanyl” refers to groups —S—R where R includes H, “C₁-C₆-alkyl”, “C₁-C₆-alkyl” optionally substituted with halogens., e.g a —S—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Amino” refers to the group —NRR′ where each R, R′ is independently hydrogen, “C₁-C₆-alkyl”, “C₂-C₆-alkenyl”, “C₂-C₆-alkynyl”, “C₃-C₈-cycloalkyl”, “Heterocycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₈-alkynyl aryl”, “C₂-C₆-alkynylheteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered hetero-cycloalkyl ring.

“Substituted or unsubstituted”: Unless otherwise constrained by the definition of the individual substituent, the above set out groups, like “alkyl”, “alkenyl”, “alkynyl”, “alkoxy”, “aryl” and “heteroaryl” etc. groups can optionally be independently substituted with from 1 to 5 substituents selected from the group consisting of “C₁-C₆-alkyl”, “C₁-C₆-alkyl aryl”, “C₁-C₆-alkyl heteroaryl”, “C₂-C₆-alkenyl”, “C₂-C₆-alkynyl”, primary, secondary or tertiary amino groups or quaternary ammonium moieties, “acyl”, “acyloxy”, “acylamino”, “aminocarbonyl”, “alkoxycarbonylamino”, “alkoxycarbonyl”, “aryl”, “aryloxy”, “heteroaryl”, “heteroaryloxy”, carboxyl, cyano, halogen, hydroxy, nitro, sulfanyl, sulphoxy, sulphonyl, sulfonamide, alkoxy, thioalkoxy, trihalomethyl and the like. Within the framework of this invention, said “substitution” is meant to also comprise situations where neighboring substituents undergo ring closure, in particular when vicinal functional substituents are involved, thus forming e.g. lactams, lactons, cyclic anhydrides, but also acetals, thioacetals, animals formed by ring closure for instance in an effort to obtain a protective group.

Where tautomerism, (e.g., keto-enol tautomerism) of compounds of the present invention or their prodrugs may occur, the individual forms (e.g., the keto, enol form, and together as mixtures in any ratio. Same applies for stereoisomers (e.g., enantiomers, cis/trans isomers, conformers and the like.)

Isomers can be separated by methods well known in the art, e.g. by liquid chromatography. Same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a “mutation” includes an amino acid residue deletion, an amino acid residue insertion, and/or an amino acid residue substitution of at least one amino acid residue in a defined primary amino acid sequence, such as a primary amino acid sequence of a target protein. An amino acid “substitution” means that at least one amino acid component of a defined primary amino acid sequence is replaced with another amino acid (for example, a cysteine residue or a lysine residue). Desirably, mutation or substitution of one or more amino acid residues (such as a conservative mutation or substitution) in a primary amino acid sequence does not result in substantial changes in the susceptibility of a target protein encoded by that amino acid sequence to undergo a conformational change upon binding to a ligand of that target protein or upon binding to an unknown candidate agent capable of allosterically binding a target protein. Methods for engineering a mutation or substitution into the primary amino acid sequence of a protein such as a target protein are well known in the art via standard techniques.

As used herein, the term “Raf kinase” refers to any one of a family of serine/threonine-protein kinases. The family consists of three isoform members (B-Raf, C-Raf (Raf-1), and A-Raf). Raf protein kinases are involved in the MAPK signaling pathway consisting of a kinase cascade that relays extracellular signals to the nucleus to regulate gene expression and key cellular functions. Unless otherwise indicated by context, the term “Raf kinase” is meant to refer to any Raf kinase protein from any species, including, without limitation. In one aspect, the Raf kinase is a human Raf kinase

The term “Raf inhibitor” or “inhibitor of Raf” is used to signify a compound which is capable of interacting with one or more isoform members (B-Raf, C-Raf (Raf-1) and/or A-Raf) of the serine/threonine-protein kinase, Raf including mutant forms. Some examples of Raf mutant forms include, but are not limited to B-Raf V600E, B-Raf V600D, B-Raf V600K, B-Raf V600E+T5291 and/or B-Raf V600E+G468A.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes place in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”, “administered”, “administers” and “administering” a compound should be understood to mean providing a compound of the disclosure or a prodrug of a compound of the disclosure to the individual in need.

The term “effective amount” or “therapeutically effective amount” refers to that amount of a compound or salt described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including, but not limited to, a therapeutic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. Treating can include, for example, reducing, delaying or alleviating the severity of one or more symptoms of the disease or condition, or it can include reducing the frequency with which symptoms of a disease, defect, disorder, or adverse condition, and the like, are experienced by a patient.

As used herein, “synergy,” “synergetic,” “synergism,” or “synergistic effect” refer to two or more compounds or compositions, that individually produce an effect, however, together produce a combined effect that is greater than their individual effects.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “about” or “approximately” can mean within an acceptable error range for the particular value as determined by one of ordinary skill in the art, which will depend in part on how the value is measured or determined, i.e. , the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The section headings used herein are for organizational purposes only and are not to be construed as limiting the subject matter described.

Any aspect or embodiment described herein can be combined with any other aspect or embodiment as disclosed herein.

### Methods of Treating a Subject Suffering from Cancer

In one aspect, the present disclosure provides methods of treating a subject suffering from cancer, comprising administering to the subject a RAF inhibitor or a pharmaceutically acceptable salt thereof and a MEK inhibitor or a pharmaceutically acceptable salt thereof. In one aspect, the present disclosure provides methods of treating a subject suffering from cancer, comprising administering to the subject a RAF inhibitor and a MEK inhibitor, wherein a total amount of the RAF inhibitor and the MEK inhibitor is therapeutically effective in treating the cancer. In one aspect, the present disclosure provides methods of treating a subject suffering from cancer, comprising administering to the subject:
(i)(R)-2-(1-(6-amino-5-chloropyrimidine-4-carboxamido)ethyl)-N-(5-chloro-4-(trifluoromethyppyridin-2-yl)thiazole-5-carboxamide (Compound A), or a pharmaceutically acceptable salt thereof and
(ii) a MEK inhibitor as provided herein;
   wherein a total amount of the Compound A or a pharmaceutically acceptable salt thereof and the MEK inhibitor is therapeutically effective in treating the cancer.
   In some embodiments, the cancer is a recurrent, progressive, or refractory solid tumor with mitogen-activated protein kinase (MAPK) pathway aberration. In some embodiments, the subject has a gene mutation or gene fusion described in Tables 1-7. In some embodiments, the cancer can not been previously treated. In some embodiments, the administration of Compound A or a pharmaceutically acceptable salt thereof and a MEK inhibitor or a pharmaceutically acceptable salt, as disclosed herein, may provide synergistic effects. In some embodiments, the synergetic effect is measured using a method as disclosed herein. In some embodiments, the synergistic effects may be determined using a mutated cell line. In some embodiments, the mutated cell line is a non V600 BRAF mutant cell line. In some embodiments, the mutated cell line is a KRAS mutant cell line. In some embodiments, the mutated cell line is a NRAS mutant cell line. In some embodiments, the synergistic effect is measured using models harboring BRAF fusions. In some embodiments, the BRAF fusions are BRAF fusions as disclosed herein. In some embodiments, the mutated cell line has a mutation provided in Table 1 or Table 2. In some embodiments, the mutated cell is a cell line provided in Table 1 or Table 2.

### A. Raf Inhibitors

In one aspect, a RAF inhibitor described herein is a B-Raf and/or C-Raf kinases inhibitor. In some embodiments, the Raf inhibitor is selective for B-Raf and C-Raf kinases. In some embodiments, the Raf inhibitor is selective for B-Raf(wild type), B-Raf V600E and C-Raf. In some embodiments, the Raf inhibitor is selective for B-Raf (wild type), B-Raf V600D and C-Rat In some embodiments, the Raf inhibitor is selective for B-Raf (wild type), B-Raf V600K and C-Raf. In some embodiments, the Raf inhibitor is selective for mutant B-Raf In some embodiments, the Raf inhibitor is selective for mutant B-Raf V600E. In some embodiments, the Raf inhibitor is selective for mutant B-Raf V600D. In some embodiments, the Raf inhibitor is selective for mutant B-Raf V600K.

Compounds capable of inhibiting the activity of a Raf kinase maybe be used in the methods of the instant disclosure. In some embodiments, the Raf inhibitor inhibits more isoforms of Raf kinase proteins than B-Raf V600. In some embodiment, the Raf inhibitor inhibits more isoforms of Raf kinase proteins than B-Raf V600E. In some embodiments, the Raf inhibitor inhibits B-Raf (wild-type), mutant B-Raf, A- Raf, and C-Raf. In some embodiments, the Raf inhibitor is selective for B-Raf (wild-type), B-Raf V600E, A-Raf and/or C-Raf In some embodiments, the Raf inhibitor is selective for B-Raf (wild-type), B- Raf V600K, A-Raf and/or C-Raf In some embodiments, the Raf inhibitor is selective for B-Raf (wild-type), B-Raf V600D, A-Raf and/or C-Raf In some embodiments, the Raf inhibitor is selective for B-Raf (wild- type), B-Raf V600K, and C-Raf In some embodiments, the Raf inhibitor is selective for B-Raf (wild-type), B-Raf V600E and C-Raf In some embodiments, the Raf inhibitor is selective for B-Raf (wild- type), B-Raf V600D and C-Raf In some embodiments, the Raf inhibitor is selective for B-Raf (wild- type), B-Raf V600K and C-Raf In some embodiments, the Raf inhibitor is selective for mutant B-Raf. In some embodiments, the Raf inhibitor is selective for mutant B-Raf V600E. In some embodiments, the Raf inhibitor is selective for mutant B-Raf V600D. In some embodiments, the Raf inhibitor is selective for mutant B-Raf V600K.

The present disclosure provides RAF inhibitors useful for the methods disclosed herein. In some embodiments, the RAF inhibitor is (R)-2-(1-(6-amino-5-chloropyrimidine-4-carboxamido)ethyl)-N-(5-chloro-4-(trifluoromethyppyridin-2-yl)thiazole-5-carboxamide (Compound A), or a pharmaceutically acceptable salt thereof. In some embodiments, the RAF inhibitor is represented by:

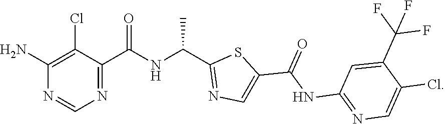 [SMILES: C[C@@H](NC(=O)c1ncnc(N)c1Cl)c1ncc(C(=O)Nc2cc(C(F)(F)F)c(Cl)cn2)s1]

In some embodiments, the RAF inhibitor is described in U.S. Pat. No. 8,293,752, which is hereby incorporated by reference in its entirety.

In some embodiments, Compound A inhibits RAF monomers and dimers without the activation of the MAPK pathway. In some embodiments, Compound A does not induce the activation of MAPK signaling in wild-type BRAF. In some embodiments, Compound A does not induce the activation of MAPK activation in BRAF fusions. In some embodiments, the BRAF fusion is KIAA1549-BRAF. In some embodiments, Compound A inhibits RAF monomers and dimers without inducing MAPK signaling.

In some embodiments, a RAF inhibitor is administered to a subject at about 50 mg to about 800 mg. In some embodiments, the RAF inhibitor is administered to a subject at about 100 mg to about 600 mg. In some embodiments, the RAF inhibitor is administered to a subject at about 100 mg to about 500 mg. In some embodiments, the RAF inhibitor is administered to a subject at about 200 mg to about 600 mg. In some embodiments, the RAF inhibitor is administered to a subject at about 50 mg, about 100 mg, about 200 mg, about 300 mg, about 400 mg, about 500 mg, about 600 mg, or about 700 mg. In some embodiments, the RAF inhibitor is administered to a subject at about 200 mg, about 400 mg, or about 600 mg. In some embodiments, the RAF inhibitor is administered to a subject at about 200 mg. In some embodiments, the RAF inhibitor is administered to a subject at about 400 mg. In some embodiments, the RAF inhibitor is administered to a subject at about 600 mg. In some embodiments, the RAF inhibitor is administered to a subject at 100 mg to 800 mg. In some embodiments, the RAF inhibitor is administered to a subject at 100 mg to 700 mg. In some embodiments, the RAF inhibitor is administered to a subject at 100 mg to 600 mg. In some embodiments, the RAF inhibitor is administered to a subject at 100 mg to 500 mg. In some embodiments, the RAF inhibitor is administered to a subject at 200 mg to 600 mg. In some embodiments, the RAF inhibitor is administered to a subject at about 50 mg, 100 mg, 200 mg, 300 mg, 400 mg, 500 mg, 600 mg, 700 mg, or 800 mg. In some embodiments, the RAF inhibitor is administered to a subject at about 200 mg, 400 mg, or 600 mg. In some embodiments, the RAF inhibitor is administered to a subject at 200 mg. In some embodiments, the RAF inhibitor is administered to a subject at 400 mg. In some embodiments, the RAF inhibitor is administered to a subject at 600 mg. In some embodiments, the subject is an adult. In some embodiments, the subject is greater than or equal to 18 years old.

In some embodiments, a RAF inhibitor (such as Compound A) is administered to a subject at about 50 mg to about 800 mg, per week. In some embodiments, the RAF inhibitor is administered to a subject at about 100 mg to about 700 mg, per week. In some embodiments, the RAF inhibitor is administered to a subject at about 100 mg to about 600 mg, per week. In some embodiments, the RAF inhibitor is administered to a subject at about 100 mg to about 500 mg, per week. In some embodiments, the RAF inhibitor is administered to a subject at about 200 mg to about 600 mg, per week. In some embodiments, the RAF inhibitor is administered to a subject at about 50 mg, about 100 mg, about 200 mg, about 300 mg, about 400 mg, about 500 mg, about 600 mg, 700 mg, or about 800 mg, per week. In some embodiments, the RAF inhibitor is administered to a subject at about 200 mg, about 400 mg, or about 600 mg, per week. In some embodiments, the RAF inhibitor is administered to a subject at about 200 mg, per week. In some embodiments, the RAF inhibitor is administered to a subject at about 400 mg, per week. In some embodiments, the RAF inhibitor is administered to a subject at about 600 mg, per week. In some embodiments, the RAF inhibitor is administered to a subject at 100 mg to 800 mg, per week. In some embodiments, the RAF inhibitor is administered to a subject at 100 mg to 700 mg, per week. In some embodiments, the RAF inhibitor is administered to a subject at 100 mg to 600 mg, per week. In some embodiments, the RAF inhibitor is administered to a subject at 100 mg to 500 mg, per week. In some embodiments, the RAF inhibitor is administered to a subject at 200 mg to 600 mg, per week. In some embodiments, the RAF inhibitor is administered to a subject at 50 mg, 100 mg, 200 mg, 300 mg, 400 mg, 500 mg, 600 mg, 700 mg, or 800 mg, per week. In some embodiments, the RAF inhibitor is administered to a subject at 200 mg, 400 mg, or 600 mg, per week. In some embodiments, the RAF inhibitor is administered to a subject at 200 mg, per week. In some embodiments, the RAF inhibitor is administered to a subject at 400 mg, per week. In some embodiments, the RAF inhibitor is administered to a subject at 600 mg, per week. In some embodiments, the per week dosing is once a week. In some embodiments, the subject is an adult. In some embodiments, the subject is greater than or equal to 18 years old.

In some embodiments, a RAF inhibitor (such as Compound A) is administered to a subject at about 100 mg/m² to about 600 mg/m². In some embodiments, the RAF inhibitor is administered to a subject at about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², about 500 mg/m², about 520 mg/m², about 540 mg/m², about 560 mg/m², about 580 mg/m², about 600 mg/m². In some embodiments, the RAF inhibitor is administered to a subject at about 140 mg/m², about 280 mg/m², or about 420 mg/m². In some embodiments, the RAF inhibitor is administered to a subject at about 140 mg/m². In some embodiments, the RAF inhibitor is administered to a subject at about 280 mg/m². In some embodiments, the RAF inhibitor is administered to a subject at about 480 mg/m². In some embodiments, the RAF inhibitor is administered to a subject at a dose of at least about 25 mg/m². In some embodiments, the RAF inhibitor is administered to a subject at 50 mg/m² to 600 mg/m². In some embodiments, the RAF inhibitor is administered to a subject at 100 mg/m² to 600 mg/m². In some embodiments, the RAF inhibitor is administered to a subject at 100 mg/m², 120 mg/m², 140 mg/m², 160 mg/m², 180 mg/m², 200 mg/m², 220 mg/m², 240 mg/m², 260 mg/m², 280 mg/m², 300 mg/m², 320 mg/m², 340 mg/m², 360 mg/m², 380 mg/m², 400 mg/m², 420 mg/m², 440 mg/m², 460 mg/m², 480 mg/m², 500 mg/m², 520 mg/m², 540 mg/m², 560 mg/m², 580 mg/m², or 600 mg/m². In some embodiments, the RAF inhibitor is administered to a subject at 140 mg/m², 280 mg/m², or 420 mg/m². In some embodiments, the RAF inhibitor is administered to a subject at 140 mg/m². In some embodiments, the RAF inhibitor is administered to a subject at 280 mg/m². In some embodiments, the RAF inhibitor is administered to a subject at 480 mg/m². In some embodiments, the subject is between 12 years old and 18 years old. In some embodiments, the subject is from about 12 years old to about 18 years old. In some embodiments, the subject is greater than or equal to 12 years old to less than or equal to 18 years. In some embodiments, the subject is younger than 12 years old. In some embodiments, the subject is at least 6 months old.

In some embodiments, a RAF inhibitor is administered to a subject at about 50 mg/m² to about 800 mg/m², per week. In some embodiments, a RAF inhibitor is administered to a subject at about 100 mg/m² to about 500 mg/m², per week. In some embodiments, the RAF inhibitor is administered to a subject at about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², or about 500 mg/m², about 520 mg/m², about 540 mg/m², about 560 mg/m², about 580 mg/m², or about 600 mg/m², per week. In some embodiments, the RAF inhibitor is administered to a subject at about 140 mg/m², about 280 mg/m², or about 420 mg/m², per week. In some embodiments, the RAF inhibitor is administered to a subject at about 140 mg/m², per week. In some embodiments, the RAF inhibitor is administered to a subject at about 280 mg/m², per week. In some embodiments, the RAF inhibitor is administered to a subject at about 480 mg/m², per week. In some embodiments, the RAF inhibitor is administered to a subject at 100 mg/m² to 600 mg/m², per week. In some embodiments, a RAF inhibitor is administered to a subject at about 100 mg/m², about 120 mg/m², about 140 mg/m², about 160 mg/m², about 180 mg/m², about 200 mg/m², about 220 mg/m², about 240 mg/m², about 260 mg/m², 280 mg/m², 300 mg/m², 320 mg/m², 340 mg/m², 360 mg/m², 380 mg/m², 400 mg/m², 420 mg/m², 440 mg/m², 460 mg/m², 480 mg/m², or 500 mg/m², 520 mg/m², 540 mg/m², 560 mg/m², 580 mg/m², or 600 mg/m², per week. In some embodiments, the RAF inhibitor is administered to a subject at about 140 mg/m², 280 mg/m², or 420 mg/m², per week. In some embodiments, the RAF inhibitor is administered to a subject at 140 mg/m², per week. In some embodiments, the RAF inhibitor is administered to a subject at 280 mg/m², per week. In some embodiments, the RAF inhibitor is administered to a subject at 480 mg/m², per week. In some embodiments, the per week dosing is once a week. In some embodiments, the subject is between 12 years old and 18 years old. In some embodiments, the subject is from about 12 years old to about 18 years old. In some embodiments, the subject is greater than or equal 12 years old to less than or equal to 18 years. In some embodiments, the subject is younger than 12 years old. In some embodiments, the subject is at least 6 months old.

In some embodiments, Compound A is administered to a subject at about 50 mg to about 800 mg. In some embodiments, Compound A is administered to a subject at about 100 mg to about 600 mg. In some embodiments, Compound A is administered to a subject at about 100 mg to about 500 mg. In some embodiments, Compound A is administered to a subject at about 200 mg to about 600 mg. In some embodiments, Compound A is administered to a subject at about 50 mg, about 100 mg, about 200 mg, about 300 mg, about 400 mg, about 500 mg, about 600 mg, 700 mg, or about 800 mg. In some embodiments, Compound A is administered to a subject at about 200 mg, about 400 mg, or about 600 mg. In some embodiments, Compound A is administered to a subject at about 200 mg. In some embodiments, Compound A is administered to a subject at about 400 mg. In some embodiments, Compound A is administered to a subject at about 600 mg. In some embodiments, Compound A is administered to a subject at 100 mg to 800 mg. In some embodiments, Compound A is administered to a subject at 100 mg to 600 mg. In some embodiments, Compound A is administered to a subject at 100 mg to 500 mg. In some embodiments, Compound A is administered to a subject at 200 mg to 600 mg. In some embodiments, Compound A is administered to a subject at 50 mg, 100 mg, 200 mg, 300 mg, 400 mg, 500 mg, 600 mg, 700 mg, or 800 mg. In some embodiments, Compound A is administered to a subject at 200 mg, 400 mg, or 600 mg. In some embodiments, Compound A is administered to a subject at 200 mg. In some embodiments, Compound A is administered to a subject at 400 mg. In some embodiments, Compound A is administered to a subject at 600 mg. In some embodiments, the subject is an adult. In some embodiments, the subject is greater than or equal to 18 years old.

In some embodiments, Compound A is administered to a subject at about 100 mg to about 800 mg, per week. In some embodiments, Compound A is administered to a subject at about 100 mg to about 600 mg, per week. In some embodiments, Compound A is administered to a subject at about 100 mg to about 500 mg, per week. In some embodiments, Compound A is administered to a subject at about 200 mg to about 600 mg, per week. In some embodiments, Compound A is administered to a subject at about 50 mg, about 100 mg, about 200 mg, about 300 mg, about 400 mg, about 500 mg, about 600 mg, or about 700 mg, about 800 mg, per week. In some embodiments, Compound A is administered to a subject at about 200 mg, about 400 mg, or about 600 mg, per week. In some embodiments, Compound A is administered to a subject at about 200 mg. In some embodiments, Compound A is administered to a subject at about 400 mg, per week. In some embodiments, Compound A is administered to a subject at about 600 mg, per week. In some embodiments, Compound A is administered to a subject at 100 mg to 700 mg, per week. In some embodiments, Compound A is administered to a subject at 100 mg to 600 mg, per week. In some embodiments, Compound A is administered to a subject at 100 mg to 500 mg, per week. In some embodiments, Compound A is administered to a subject at 200 mg to 600 mg, per week. In some embodiments, Compound A is administered to a subject at 50 mg, 100 mg, 200 mg, 300 mg, 400 mg, 500 mg, 600 mg, or 700 mg, per week. In some embodiments, Compound A is administered to a subject at 200 mg, 400 mg, or 600 mg, per week. In some embodiments, Compound A is administered to a subject at 200 mg, per week. In some embodiments, Compound A is administered to a subject at 400 mg, per week. In some embodiments, Compound A is administered to a subject at 600 mg, per week. In some embodiments, the per week dosing is once a week. In some embodiments, the subject is an adult. In some embodiments, the subject is greater than or equal to 18 years old.

In some embodiments, Compound A is administered to a subject at about 500 mg/m² to about 800 mg/m². In some embodiments, Compound A is administered to a subject at about 100 mg/m² to about 500 mg/m². In some embodiments, Compound A is administered to a subject at about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², or about 500 mg/m², about 520 mg/m², about 540 mg/m², about 560 mg/m², about 580 mg/m², or about 600 mg/m². In some embodiments, Compound A is administered to a subject at about 140 mg/m², about 280 mg/m², or about 420 mg/m². In some embodiments, Compound A is administered to a subject at about 140 mg/m². In some embodiments, Compound A is administered to a subject at about 280 mg/m². In some embodiments, Compound A is administered to a subject at about 480 mg/m². In some embodiments, Compound A is administered to a subject at 100 mg/m² to 500 mg/m². In some embodiments, Compound A is administered to a subject at 100 mg/m², 120 mg/m², 140 mg/m², 160 mg/m², 180 mg/m², 200 mg/m², 220 mg/m², 240 mg/m², 260 mg/m², 280 mg/m², 300 mg/m², 320 mg/m², 340 mg/m², 360 mg/m², 380 mg/m², 400 mg/m², 420 mg/m², 440 mg/m², 460 mg/m², 480 mg/m², or 500 mg/m², 520 mg/m², 540 mg/m², 560 mg/m², 580 mg/m², or 600 mg/m². In some embodiments, Compound A is administered to a subject at 140 mg/m², 280 mg/m², or 420 mg/m². In some embodiments, Compound A is administered to a subject at 140 mg/m². In some embodiments, Compound A is administered to a subject at 280 mg/m². In some embodiments, Compound A is administered to a subject at 480 mg/m². In some embodiments, the subject is between 12 years old and 18 years old. In some embodiments, the subject is from about 12 years old to 18 years old. In some embodiments, the subject is greater than or equal 12 years old and less than or equal to 18 years. In some embodiments, the subject is younger than 12 years old. In some embodiments, the subject is at least 6 months old.

In some embodiments, Compound A is administered to a subject at about 100 mg/m² to about 500 mg/m², per week. In some embodiments, Compound A is administered to a subject at about 100 mg/m², about 120 mg/m², about 140 mg/m², about 160 mg/m², about 180 mg/m², about 200 mg/m², about 220 mg/m², about 240 mg/m², about 260 mg/m², about 280 mg/m², about 300 mg/m², about 320 mg/m², about 340 mg/m², about 360 mg/m², about 380 mg/m², about 400 mg/m², about 420 mg/m², about 440 mg/m², about 460 mg/m², about 480 mg/m², or about 500 mg/m², about 520 mg/m², about 540 mg/m², about 560 mg/m², about 580 mg/m², or about 600 mg/m², per week. In some embodiments, Compound A is administered to a subject at about 140 mg/m², about 280 mg/m², or about 420 mg/m², per week. In some embodiments, Compound A is administered to a subject at about 140 mg/m², per week. In some embodiments, Compound A is administered to a subject at about 280 mg/m², per week. In some embodiments, Compound A is administered to a subject at about 480 mg/m², per week. In some embodiments, Compound A is administered to a subject at 100 mg/m² to 500 mg/m², per week. In some embodiments, Compound A is administered to a subject at 100 mg/m², 120 mg/m², 140 mg/m², 160 mg/m², 180 mg/m², 200 mg/m², 220 mg/m², 240 mg/m², 260 mg/m², 280 mg/m², 300 mg/m², 320 mg/m², 340 mg/m², 360 mg/m², 380 mg/m², 400 mg/m², 420 mg/m², 440 mg/m², 460 mg/m², 480 mg/m², or 500 mg/m², 520 mg/m², 540 mg/m², 560 mg/m², 580 mg/m², or 600 mg/m², per week. In some embodiments, Compound A is administered to a subject at 140 mg/m², 280 mg/m², or 420 mg/m², per week. In some embodiments, Compound A is administered to a subject at 140 mg/m², per week. In some embodiments, Compound A is administered to a subject at 280 mg/m², per week. In some embodiments, Compound A is administered to a subject at 480 mg/m², per week. In some embodiments, the per week dosing is once a week. In some embodiments, the subject is between 12 years old and 18 years old. In some embodiments, the subject is between greater than or equal 12 years old and less than or equal to 18 years. In some embodiments, the subject is younger than 12 years old. In some embodiments, the subject is at least 6 months old.

In some embodiments, Compound A is administered at a dose once weekly (QW). In some embodiments, the administered dose once weekly (QW) of Compound A is higher than 600 mg. In some embodiments, the administered dose once weekly (QW) of Compound A is at most 600 mg. In some embodiments, the administered dose once weekly (QW) of Compound is at most 530 mg. In some embodiments, the administered dose once weekly (QW) of Compound A is at most 420 mg. In some embodiments, the administered dose once weekly (QW), is at most 350 mg. In some embodiments, the administered dose once weekly (QW) of Compound A is at most 280 mg. In some embodiments, the administered dose once weekly (QW) of Compound A is 600 mg. In some embodiments, the administered dose once weekly (QW) of Compound A is 530 mg. In some embodiments, the administered dose once weekly (QW) of Compound A is 420 mg. In some embodiments, the administered dose once weekly (QW), is 350 mg. In some embodiments, the administered dose once weekly (QW) of Compound A is 280 mg.

In some embodiments, Compound A is orally administered (PO) up to a maximum dose once weekly (QW). In some embodiments, the maximum oral dose (PO) administered once weekly (QW) of Compound A is higher than 600 mg. In some embodiments, the maximum oral dose (PO) administered once weekly (QW) of Compound A is at most 600 mg. In some embodiments, the maximum oral dose (PO) administered once weekly (QW) of Compound A is at most 530 mg. In some embodiments, the maximum oral dose (PO) administered once weekly (QW) of Compound A is at most 420 mg. In some embodiments, the maximum oral dose (PO) administered once weekly (QW), is at most 350 mg. In some embodiments, the maximum oral dose (PO) administered once weekly (QW) of Compound A is at most 280 mg. In some embodiments, the maximum oral dose (PO) administered once weekly (QW) of Compound A is 600 mg. In some embodiments, the maximum oral dose (PO) administered once weekly (QW) of Compound A is 530 mg. In some embodiments, the maximum oral dose (PO) administered once weekly (QW) of Compound A is 420 mg. In some embodiments, the maximum oral dose (PO) administered once weekly (QW) of Compound A is 350 mg. In some embodiments, the maximum oral dose (PO) administered once weekly (QW) of Compound A is 280 mg. In some embodiments, the Compound A or a pharmaceutically acceptable salt or solvate thereof is Compound A.

In some embodiments, about 140 mg/m² of Compound A is chronically administered once a week to the subject. In some embodiments, about 280 mg/m² of Compound A is chronically administered once a week to the subject. In some embodiments, about 350 mg/m² of Compound A is chronically administered once a week to the subject. In some embodiments, about 420 mg/m² of Compound A is chronically administered once a week to the subject. In some embodiments, about 530 mg/m² of Compound A is chronically administered once a week to the subject. In some embodiments, about 140 mg/m² of Compound A is chronically administered once a week over the course of 360 days. In some embodiments, about 280 mg/m² of Compound A is chronically administered once a week over the course of 360 days. In some embodiments, about 350 mg/m² of Compound A is chronically administered once a week over the course of 360 days. In some embodiments, about 420 mg/m² of Compound A is chronically administered once a week over the course of 360 days. In some embodiments, about 530 mg/m² of Compound A is chronically administered once a week over the course of 360 days. .In some embodiments, about 140 mg/m² of Compound A is chronically administered once a week for at least 1 year. In some embodiments, about 280 mg/m² of Compound A is chronically administered once a week for at least 1 year. In some embodiments, about 350 mg/m² of Compound A is chronically administered once a week for at least 1 year. In some embodiments, about 420 mg/m² of Compound A is chronically administered once a week for at least 1 year. In some embodiments, about 530 mg/m² of Compound A is chronically administered once a week for at least 1 year.

In some embodiments, about 200 mg of Compound A is chronically administered once a week to the subject. In some embodiments, about 400 mg of Compound A is chronically administered once a week to the subject. In some embodiments, about 600 mg of Compound A is chronically administered once a week to the subject. In some embodiments, about 200 mg of Compound A is chronically administered once a week over the course of 360 days. In some embodiments, about 400 mg of Compound A is chronically administered once a week over the course of 360 days. In some embodiments, about 420 mg/m² of Compound A is chronically administered once a week over the course of 360 days. In some embodiments, about 600 mg of Compound A is chronically administered once a week over the course of 360 days. In some embodiments, about 200 mg of Compound A is chronically administered once a week for at least 1 year. In some embodiments, about 400 mg of Compound A is chronically administered once a week for at least 1 year. In some embodiments, about 600 mg of Compound A is chronically administered once a week for at least 1 year.

In some embodiments, the dosing described herein for Compound A or a salt thereof is based on the weight of Compound A. In some embodiments, the dosing described herein for Compound A or a salt thereof corresponds to the weight of the free base form of Compound A. For example, in some embodiments, the dosing of the Compound A or a salt thereof describes the weight of the Compound A in such dosing. In some embodiments, the dosing described herein for Compound A or a salt thereof corresponds to the weight of the salt of Compound A.

### B. MEK Inhibitors

The present disclosure provides MEK inhibitors for the methods as disclosed herein. In some embodiments, the MEK inhibitor is a compound having a structure of Formula (I) or a pharmaceutically acceptable salt thereof,

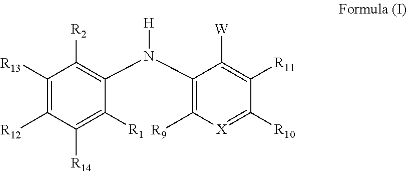

wherein,
R₁, R₂, R₉, R₁₀, R₁₁ R₁₂, R₁₃ and R₁₄ are independently selected from: hydrogen, halogen, cyano, nitro, azido, —OR₃, —NR₄C(O)OR₆, —OC(O)R₃, —NR₄S(O)ⱼR₆, —S(O)ⱼNR₃R₄, —S(O)ⱼNR₄C(O)R₃, —C(O)NR₄S(O)ⱼR₆, —S(O)ⱼR₆, —NR₄C(O)R₃, —C(O)NR₃R₄, —NR₅C(O)NR₃R₄, —NR₅C(NCN)NR₃R₄, —NR₃R₄, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR₄R₅)ₘ-aryl, aryl, arylalkyl, heteroaryl, heteroarylalkyl, heterocyclyl, heterocyclylalkyl, —O(CR₄R₅)ₘ-aryl, —NR₄(CR₄R₅)ₘ-aryl, —O(CR₄R₅)ₘ-heteroaryl, —NR₄(CR₄R₅)ₘ, heteroaryl, —O(CR₄R₅)ₘ-heterocyclyl, —NR₄(CR₄R₅)ₘ-heterocyclyl and —S(C₁-C₂ alkyl) substituted with 1 to 5 fluorines, wherein each alkyl, alkenyl, alkynyl, cycloalkyl, aryl, heteroaryl and heterocyclyl is independently substituted or unsubstituted;
R₃ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, arylalkyl, heteroaryl, heteroarylalkyl, heterocyclyl, heterocyclylalkyl, and aryl; wherein each alkyl, alkenyl, alkynyl, cycloalkyl, heteroaryl and heterocyclyl is independently substituted or unsubstituted; and wherein aryl is optionally substituted with 1 to 5 groups independently selected from: oxo, halogen, nitro, CF₃, CHF₂, CH₂F, OCF₃, OCHF₂, OCH₂F, azido, NR′SO₂R″″, SO₂N″, C(O)R′, C(O)OR′, OC(0)R′, NR′C(O)OR″″, NR′C(O)R″, C(O)NR′R″, SR″″, S(0)R″″, SO₂R′, NR′R″, NR′C(O)NR″R′″, NR′C(NCN)N″R″′, OR′, aryl, heteroaryl, arylalkyl, heteroarylalkyl, heterocyclyl, and heterocyclylalkyl;
R₄ is selected from hydrogen or C₁₋₆ alkyl, wherein alkyl may be substituted or unsubstituted; or
R₃ and R₄ can be taken together with the atom to which they are attached to form a 4 to 10 membered heteroaryl or heterocyclic ring, each of which is substituted or unsubstituted;
R₅ is hydrogen or C₁-C₆ alkyl, wherein alkyl may be substituted or unsubstituted; or
R₄ and R₅ can be taken together with the atom to which they are attached to form a 4 to 10 membered carbocyclic, heteroaryl or heterocyclic ring, each of which is substituted or unsubstituted;
R₆ is selected from: trifluoromethyl, C₁-C₁₀ alkyl, C₃-C₁₀ cycloalkyl, aryl, arylalkyl, heteroaryl, heteroarylalkyl, heterocyclyl, and heterocyclylalkyl, wherein each alkyl, cycloalkyl, aryl, heteroaryl and heterocyclyl is independently substituted or unsubstituted;
R′, R″ and R″′ are independently selected from: hydrogen, C₁-C₄ alkyl, C₂-C₄ alkenyl, aryl and arylalkyl;
R″″ is selected from C₁-C₄ alkyl, C₂-C₄ alkenyl, aryl and arylalkyl;
W is selected from 1) heteroaryl containing 1-4 heteroatoms or herterocyclyl containing 1-4 heteroatoms each of which is unsubstituted or substituted by 1 to 5 substituents ZR₁₅; and 2) —C(O)OR₁₅, —C(O)NR₄R₁₅, —C(O)NR₄OR₁₅, —C(O)NR₄S(O)ⱼR₆, —C(O)NR₄NR₄NR₁₅, —NR′R″, —NR′C(O)R′, —NR′S(O)ⱼR′, —NRC(O)NR′R″, NR′S(O)ⱼNR′R″, or —C(O)NR₄NR₄C(O)R₁₅; provided that W is not —C(O)OH;
Z is a bond, NR₁₆,O, NR₁₆SO₂ or S;
R₁₅ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl; wherein each alkyl, alkenyl, alkynyl, cycloalkyl, aryl, heteroaryl and heterocyclyl is independently substituted or unsubstituted;
R₁₆ is selected from hydrogen or C₁-C₁₀ alkyl; or R₁₅ and R₁₆ taken together with the atom to which they are attached form a 4 to 10 membered cyclic ring with 1 or 2 nitrogen atoms and optionally an oxygen atom, said ring being substituted or unsubstituted;
X is N or N⁺O⁻;
m is 0, 1, 2, 3,4 or 5; and
j is 1 or 2;
   wherein a total amount of the Compound A or a pharmaceutically acceptable salt thereof and the MEK inhibitor is therapeutically effective in treating the cancer. In some embodiments, the MEK inhibitor is not 3-(4-methoxy-phenylamino)-isonicotinic acid, 3-phenylamino-isonicotinic acid methyl ester, 2,3,6-trifluoro-5-phenylamino-isonicotinic acid, or 3-oxo-3-(3-phenylamino-pyridin-4-yl)-propionic acid ethyl ester.

In some embodiments of a compound having a structure of Formula (I) or a pharmaceutically acceptable salt thereof, Ri is selected from H and F. In some embodiments, R₁ is H.

In some embodiments of a compound having a structure of Formula (I) or a pharmaceutically acceptable salt thereof, R₂ is selected from: hydrogen, F, Cl, and Me; wherein the methyl group is optionally substituted with one to three fluorines. In some embodiments, R₂ is F.

In some embodiments of a compound having a structure of Formula (I) or a pharmaceutically acceptable salt thereof, R₉ is selected from: H, F, and Cl. In some embodiments, R₉ is H.

In some embodiments of a compound having a structure of Formula (I) or a pharmaceutically acceptable salt thereof, R₁₀ is selected from: H, F, Cl, Br, nitro, —SO₂NR₃R₄ or —C(O)NR₃R₄, —Me, and —OMe, wherein the methyl groups are optionally substituted with one to three fluorines. In some embodiments, R₁₀ is H.

In some embodiments of a compound having a structure of Formula (I) or a pharmaceutically acceptable salt thereof, R₁₁ is selected from: H, F, Cl, Br, Me, and —OMe; wherein the methyl groups are optionally substituted with one to three fluorines. In some embodiments, R₁₁ is H.

In some embodiments of a compound having a structure of Formula (I) or a pharmaceutically acceptable salt thereof, R₁₂ is selected from: H, F, Cl, Br, nitro, Me, —SCF₃, —SCHF₂, —SCH₂F, —SO₂NR₃R₄, —C(O)NR₃R₄ and —OMe; wherein the methyl groups are optionally substituted with one to three fluorines. In some embodiments, R₁₂ is I.

In some embodiments of a compound having a structure of Formula (I) or a pharmaceutically acceptable salt thereof, R₁₃ is H or F. In some embodiments, R₁₃ is H. In some embodiments of a compound having a structure of Formula (I) or a pharmaceutically acceptable salt thereof, R₁₄ is H or F. In some embodiments, R₁₄ is H.

In some embodiments of a compound having a structure of Formula (I) or a pharmaceutically acceptable salt thereof, W is —C(O)OR₁₅, —C(O)NR₄R₁₅, —C(O)NR₄OR₁₅, —C(O)(C₂-C₁₀ alkyl), or —C(O)NR₄S(O)ⱼR₆. In some embodiments, W is —C(O)NHR₁₅. In some embodiments, R₁₅ is C₁-C₄ alkyl or C₁-C₄ alkenyl; wherein each is independently and optionally substituted with 1 to 3 —OH, —OMe, —NH₂, —N(methyl)₂ or —N(ethyl)₂. In some embodiments, R₁₅ is C₁-C₄ alkyl substituted with 1 to 3 —OH. In some embodiments, W is

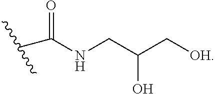 [SMILES: *C(=O)NCC(O)CO]

In some embodiments, W is

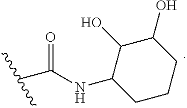 [SMILES: *C(=O)NC1CCCC(O)C1O]

In some embodiments of a compound having a structure of Formula (I) or a pharmaceutically acceptable salt thereof, X is N.

In some embodiments, the MEK inhibitor is a compound having a structure of Formula (Ia) or a pharmaceutically acceptable salt thereof:

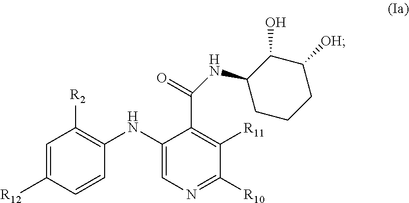

wherein,
R₂, R₁₂, R₁₀, and R₁₁ are independently selected from: hydrogen, halogen, cyano, nitro, azido, —OR₃, —NR₄C(O)OR₆, —OC(O)R₃, —NR₄S(O)ⱼR₆, —S(O)ⱼNR₃R₄, —S(O)ⱼNR₄C(O)R₃, —C(O)NR₄S(O)ⱼR₆, —S(O)ⱼR₆, —NR₄C(O)R₃, —C(O)NR₃R₄, —NR₅C(O)NR₃R₄, —NR₅C(NCN)NR₃R₄, —NR₃R₄, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR₄R₅)ₘ-aryl, aryl, arylalkyl, heteroaryl, heteroarylalkyl, heterocyclyl, heterocyclylalkyl, —O(CR₄R₅)ₘ-aryl, —NR₄(CR₄R₅)ₘ-aryl, —O(CR₄R₅)ₘ-heteroaryl, —NR₄(CR₄R₅)ₘ, heteroaryl, —(CR₄R₅)ₘ-heterocyclyl, —NR₄(CR₄R₅)ₘ-heterocyclyl and —S(C₁-C₂ alkyl) substituted with 1 to 5 fluorines, wherein each alkyl, alkenyl, alkynyl, cycloalkyl, aryl, heteroaryl and heterocyclyl is independently substituted or unsubstituted;
R₃ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, arylalkyl, heteroaryl, heteroarylalkyl, heterocyclyl, heterocyclylalkyl, and aryl; wherein each alkyl, alkenyl, alkynyl, cycloalkyl, heteroaryl and heterocyclyl is independently substituted or unsubstituted; and wherein aryl is optionally substituted with 1 to 5 groups independently selected from: oxo, halogen, nitro, CF₃, CHF₂, CH₂F, OCF₃, OCHF₂, OCH₂F, azido, NR′SO₂R″″, SO₂N″, C(O)R′, C(O)OR′, OC(0)R′, NR′C(O)OR″″, NR′C(O)R″, C(O)NR′R″, SR″″, S(0)R″″, SO₂R′, NR′R″, NR′C(O)NR″R′″, NR′C(NCN)N″R″′, OR′, aryl, heteroaryl, arylalkyl, heteroarylalkyl, heterocyclyl, and heterocyclylalkyl;
R₄ is selected from hydrogen or C₁₋₆ alkyl, wherein alkyl may be substituted or unsubstituted; or
R₃ and R₄ can be taken together with the atom to which they are attached to form a 4 to 10 membered heteroaryl or heterocyclic ring, each of which is substituted or unsubstituted;
R₅ is hydrogen or C₁-C₆ alkyl, wherein alkyl may be substituted or unsubstituted; or
R₄ and R₅ can be taken together with the atom to which they are attached to form a 4 to 10 membered carbocyclic, heteroaryl or heterocyclic ring, each of which is substituted or unsubstituted;
R₆ is selected from: trifluoromethyl, C₁-C₁₀ alkyl, C₃-C₁₀ cycloalkyl, aryl, arylalkyl, heteroaryl, heteroarylalkyl, heterocyclyl, and heterocyclylalkyl, wherein each alkyl, cycloalkyl, aryl, heteroaryl and heterocyclyl is independently substituted or unsubstituted;
R′, R″ and R″′ are independently selected from: hydrogen, C₁-C₄ alkyl, C₂-C₄ alkenyl, aryl and arylalkyl;
R″″ is selected from C₁-C₄ alkyl, C₂-C₄ alkenyl, aryl and arylalkyl;
m is 0, 1, 2, 3,4 or 5; and
j is 1 or 2.

In some embodiments, the MEK inhibitor is a compound having a structure of Formula (Ia) or a pharmaceutically acceptable salt thereof:

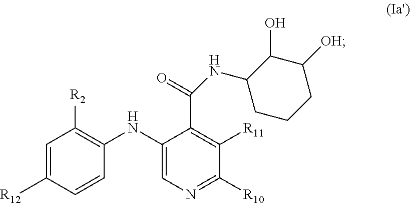

wherein,
R₂, R₁₂, R₁₀, and R₁₁ are independently selected from: hydrogen, halogen, cyano, nitro, azido, —OR₃, —NR₄C(O)OR₆, —OC(O)R₃, —NR₄S(O)ⱼR₆, —S(O)ⱼNR₃R₄, —S(O)ⱼNR₄C(O)R₃, —C(O)NR₄S(O)ⱼR₆, —S(O)ⱼR₆, —NR₄C(O)R₃, —C(O)NR₃R₄, —NR₅C(O)NR₃R₄, —NR₅C(NCN)NR₃R₄, —NR₃R₄, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR₄R₅)ₘ-aryl, aryl, arylalkyl, heteroaryl, heteroarylalkyl, heterocyclyl, heterocyclylalkyl, —(CR₄R₅)ₘ-aryl, —NR₄(CR₄R₅)ₘ-aryl, —O(CR₄R₅)ₘ-heteroaryl, —NR₄(CR₄R₅)ₘ, heteroaryl, —O(CR₄R₅)ₘ-heterocyclyl, —NR₄(CR₄R₅)ₘ-heterocyclyl and —S(C₁-C₂ alkyl) substituted with 1 to 5 fluorines, wherein each alkyl, alkenyl, alkynyl, cycloalkyl, aryl, heteroaryl and heterocyclyl is independently substituted or unsubstituted;
R₃ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, arylalkyl, heteroaryl, heteroarylalkyl, heterocyclyl, heterocyclylalkyl, and aryl; wherein each alkyl, alkenyl, alkynyl, cycloalkyl, heteroaryl and heterocyclyl is independently substituted or unsubstituted; and wherein aryl is optionally substituted with 1 to 5 groups independently selected from: oxo, halogen, nitro, CF₃, CHF₂, CH₂F, OCF₃, OCHF₂, OCH₂F, azido, NR′SO₂R″″, SO₂N″, C(O)R′, C(O)OR′, OC(0)R′, NR′C(O)OR″″, NR′C(O)R″, C(O)NR′R″, SR″″, S(0)R″″, SO₂R′, NR′R″, NR′C(O)NR″R′″, NR′C(NCN)N″R″′, OR′, aryl, heteroaryl, arylalkyl, heteroarylalkyl, heterocyclyl, and heterocyclylalkyl;
R₄ is selected from hydrogen or C₁₋₆ alkyl, wherein alkyl may be substituted or unsubstituted; or
R₃ and R₄ can be taken together with the atom to which they are attached to form a 4 to 10 membered heteroaryl or heterocyclic ring, each of which is substituted or unsubstituted;
R₅ is hydrogen or C₁-C₆ alkyl, wherein alkyl may be substituted or unsubstituted; or
R₄ and R₅ can be taken together with the atom to which they are attached to form a 4 to 10 membered carbocyclic, heteroaryl or heterocyclic ring, each of which is substituted or unsubstituted;
R₆ is selected from: trifluoromethyl, C₁-C₁₀ alkyl, C₃-C₁₀ cycloalkyl, aryl, arylalkyl, heteroaryl, heteroarylalkyl, heterocyclyl, and heterocyclylalkyl, wherein each alkyl, cycloalkyl, aryl, heteroaryl and heterocyclyl is independently substituted or unsubstituted;
R′, R″ and R″′ are independently selected from: hydrogen, C₁-C₄ alkyl, C₂-C₄ alkenyl, aryl and arylalkyl;
R″″ is selected from C₁-C₄ alkyl, C₂-C₄ alkenyl, aryl and arylalkyl;
m is 0, 1, 2, 3,4 or 5; and
j is 1 or 2.

In some embodiments of a compound having a structure of Formula (Ia), Formula (Ia′) or a pharmaceutically acceptable salt thereof, R₂ is selected from: hydrogen, F, Cl, and Me; wherein the methyl group is optionally substituted with one to three fluorines. In some embodiments, R₂ is F.

In some embodiments of a compound having a structure of Formula (Ia), Formula (Ia′) or a pharmaceutically acceptable salt thereof, R₁₂ is selected from: H, F, Cl, Br, nitro, Me, —SCF₃, —SCHF₂, —SCH₂F, —SO₂NR₃R₄, —C(O)NR₃R₄ and —OMe; wherein the methyl groups are optionally substituted with one to three fluorines. In some embodiments, R₁₂ is I.

In some embodiments of a compound having a structure of Formula (Ia), Formula (Ia′) or a pharmaceutically acceptable salt thereof, R₁₀ is selected from: H, F, Cl, Br, nitro, —SO₂NR₃R₄ or —C(O)NR₃R₄, —Me, and —OMe, wherein the methyl groups are optionally substituted with one to three fluorines. In some embodiments, R₁₀ is H.

In some embodiments of a compound having a structure of Formula (Ia), Formula (Ia′) or a pharmaceutically acceptable salt thereof, R₁₁ is selected from: H, F, Cl, Br, Me, and —OMe; wherein the methyl groups are optionally substituted with one to three fluorines. In some embodiments, R₁₁ is H.

In some embodiments, the MEK inhibitor is a compound having a structure of Formula (Ib) or a pharmaceutically acceptable salt thereof:

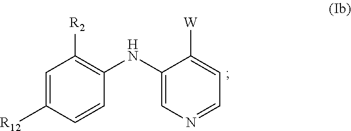

wherein,
R₂ and R₁₂, are independently selected from: hydrogen, halogen, cyano, nitro, azido, —OR₃, —C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl, wherein each alkyl, alkenyl, alkynyl, cycloalkyl, aryl, heteroaryl and heterocyclyl is independently substituted or unsubstituted;
W is selected from —C(O)NR₄R₁₅, —C(O)NR₄OR₁₅, —C(O)NR₄NR₄NR₁₅, —NR′R″, —NR′C(O)R′, —NRC(O)NR′R″, or —C(O)NR₄NR₄C(O)R₁₅;
R₃ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, arylalkyl, heteroaryl, heteroarylalkyl, heterocyclyl, heterocyclylalkyl, and aryl; wherein each alkyl, alkenyl, alkynyl, cycloalkyl, heteroaryl and heterocyclyl is independently substituted or unsubstituted; and wherein aryl is optionally substituted with 1 to 5 groups independently selected from: oxo, halogen, nitro, CF₃, CHF₂, CH₂F, OCF₃, OCHF₂, OCH₂F, azido, NR′SO₂R″″, SO₂N″, C(O)R′, C(O)OR′, OC(0)R′, NR′C(O)OR″″, NR′C(O)R″, C(O)NR′R″, SR″″, S(0)R″″, SO₂R′, NR′R″, NR′C(O)NR″R′″, NR′C(NCN)N″R″′, OR′, aryl, heteroaryl, arylalkyl, heteroarylalkyl, heterocyclyl, and heterocyclylalkyl;
R₄ is selected from hydrogen or C₁₋₆ alkyl, wherein alkyl may be substituted or unsubstituted;
R₁₅ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl; wherein each alkyl, alkenyl, alkynyl, cycloalkyl, aryl, heteroaryl and heterocyclyl is independently substituted or unsubstituted;
R′, R″ and R″′ are independently selected from: hydrogen, C₁-C₄ alkyl, C₂-C₄ alkenyl, aryl and arylalkyl; and
R″″ is selected from C₁-C₄ alkyl, C₂-C₄ alkenyl, aryl and arylalkyl.

In some embodiments of a compound having a structure of Formula (Ib) or a pharmaceutically acceptable salt thereof, R₂ is selected from: hydrogen, F, Cl, and Me; wherein the methyl group is optionally substituted with one to three fluorines. In some embodiments, R₂ is F.

In some embodiments of a compound having a structure of Formula (Ib) or a pharmaceutically acceptable salt thereof, R₁₂ is selected from: H, F, Cl, Br, nitro, Me, —SCF₃, —SCHF₂, —SCH₂F, —SO₂NR₃R₄, —C(O)NR₃R₄ and —OMe; wherein the methyl groups are optionally substituted with one to three fluorines. In some embodiments, R₁₂ is I.

In some embodiments of a compound having a structure of Formula (Ib) or a pharmaceutically acceptable salt thereof, W is —C(O)OR₁₅, —C(O)NR₄R₁₅, —C(O)NR₄OR₁₅, —C(O)(C₂-C₁₀ alkyl), or —C(O)NR₄S(O)ⱼR₆. In some embodiments, W is —C(O)NHR₁₅. In some embodiments, R₁₅ is C₁-C₄ alkyl or C₁-C₄ alkenyl; wherein each is independently and optionally substituted with 1 to 3 —OH, —OMe, —NH₂, —N(methyl)₂ or —N(ethyl)₂. In some embodiments, R₁₅ is C₁-C₄ alkyl substituted with 1 to 3 —OH. In some embodiments, W is

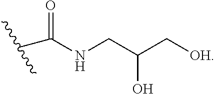 [SMILES: *C(=O)NCC(O)CO]

In some embodiments, the MEK inhibitor is

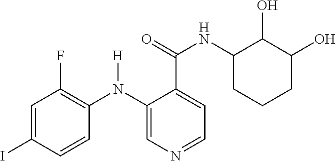 [SMILES: O=C(NC1CCCC(O)C1O)c1ccncc1Nc1ccc(I)cc1F]

or a pharmaceutically acceptable salt thereof. In some embodiments, the MEK inhibitor is

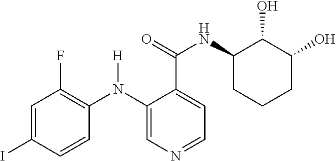 [SMILES: O=C(N[C@@H]1CCC[C@@H](O)[C@H]1O)c1ccncc1Nc1ccc(I)cc1F]

or a pharmaceutically acceptable salt thereof. In some embodiments, the MEK inhibitor or a salt thereof is N-[(2S)-2,3-dihydroxypropyl]-3-[(2-fluoro-4-iodophenyl) amino] isonicotinamide hydrochloride (i.e., N-[(2S)-2,3-dihydroxypropyl]-3-[(2-fluoro-4-iodophenyl) amino] pyridine-4-carboxamide hydrochloride). In some embodiments, the MEK inhibitor or a salt thereof has a structure of

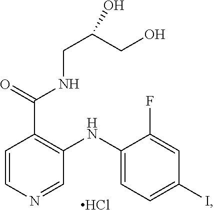 [SMILES: O=C(NC[C@H](O)CO)c1ccncc1Nc1ccc(I)cc1F ; Cl]

In some embodiments, the MEK inhibitor is N-((1R,2S,3R)-2,3-dihydroxycyclohexyl)-3-((2-fluoro-4-iodophenyl)amino)isonicotinamide, or a pharmaceutically acceptable salt thereof.

In some embodiments, the MEK inhibitor is

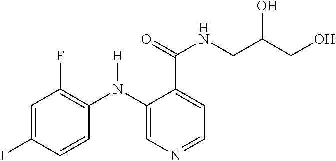 [SMILES: O=C(NCC(O)CO)c1ccncc1Nc1ccc(I)cc1F]

or a pharmaceutically acceptable salt thereof. In some embodiments, the MEK inhibitor is

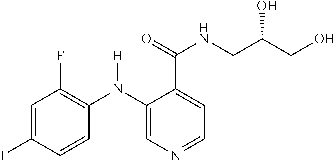 [SMILES: O=C(NC[C@H](O)CO)c1ccncc1Nc1ccc(I)cc1F]

or a pharmaceutically acceptable salt thereof. In some embodiments, the MEK inhibitor is N-((S)-2,3-Dihydroxy-propyl)-3-(2-fluoro-4-iodo-phenylamino)-isonicotinamide (pimasertib) or a pharmaceutically acceptable salt thereof.

In some embodiments, the MEK inhibitor or a pharmaceutically acceptable salt thereof is selected from cobimetinib, selumetinib, pimasertib, PD0325901, refametinib, binimetinib, BI-847325, trametinib, GDC-0623, G-573, CH5126766, CI-1040, PD035901 and TAK-933. In some embodiments, the MEK inhibitor or a pharmaceutically acceptable salt thereof is selected from cobimetinib, selumetinib, pimasertib, PD0325901, refametinib, binimetinib, BI-847325, trametinib, GDC-0623, G-573, CH5126766, CI-1040, PD035901, TAK-933, and CIP-137401. In some embodiments, the MEK inhibitor is selected from:

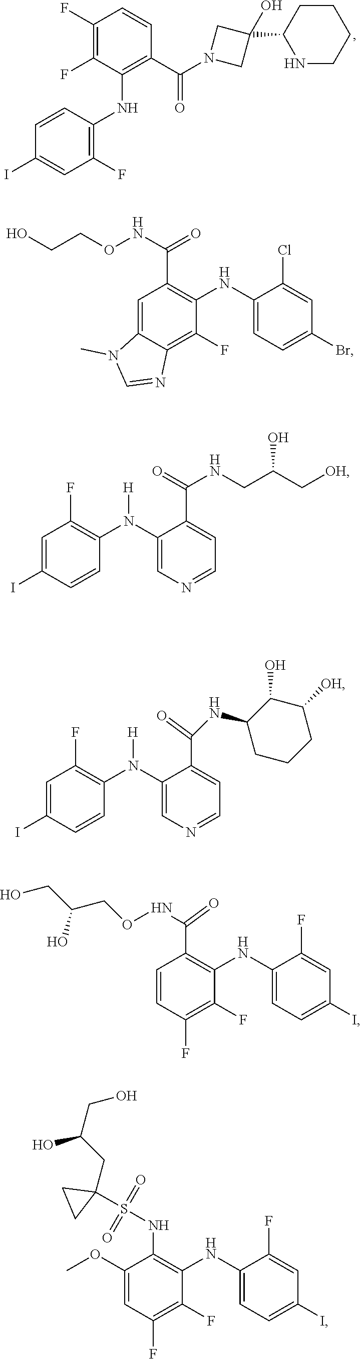 [SMILES: O=C(c1ccc(F)c(F)c1Nc1ccc(I)cc1F)N1CC(O)([C@@H]2CCCCN2)C1 ; Cn1cnc2c(F)c(Nc3ccc(Br)cc3Cl)c(C(=O)NOCCO)cc21 ; O=C(NC[C@H](O)CO)c1ccncc1Nc1ccc(I)cc1F ; O=C(N[C@@H]1CCC[C@@H](O)[C@H]1O)c1ccncc1Nc1ccc(I)cc1F ; O=C(NOC[C@H](O)CO)c1ccc(F)c(F)c1Nc1ccc(I)cc1F ; COc1cc(F)c(F)c(Nc2ccc(I)cc2F)c1NS(=O)(=O)C1(C[C@@H](O)CO)CC1]

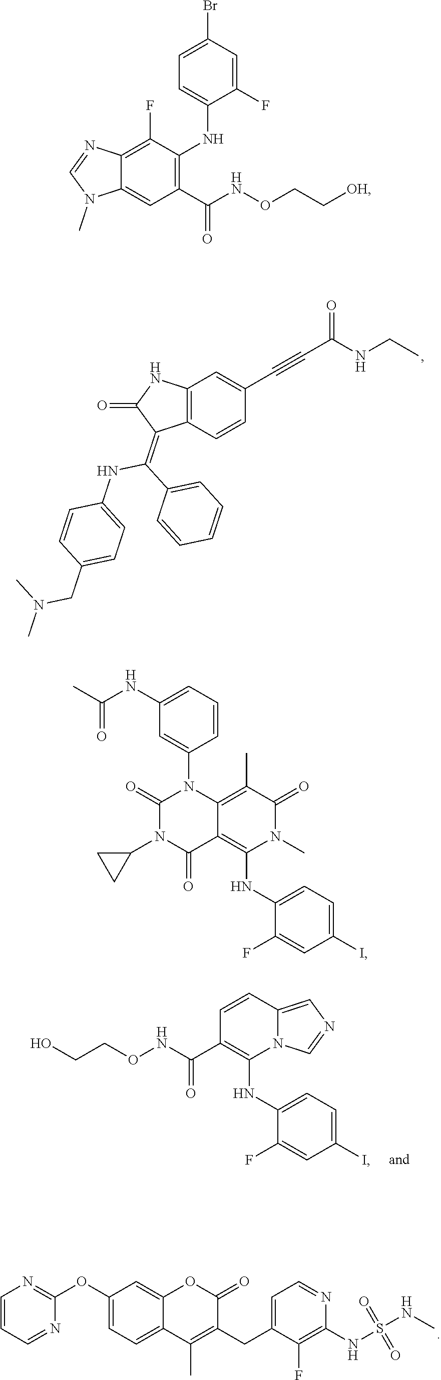

In some embodiments, the MEK inhibitor is a MEK inhibitor as described in U.S. Pat. Nos. 7,777,050, 8,178,693, 9,562,016, 7,425,637, 8,178,693, 9,156,795, 9,562,017, 7,378,423, 8,703,781, 9,290,468, each of which are hereby individually incorporated by reference in their entirety. In some embodiments, the MEK inhibitor is selected from:

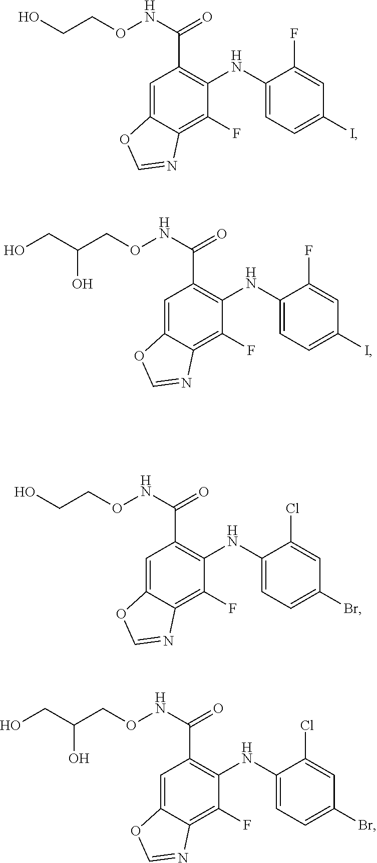 [SMILES: O=C(NOCCO)c1cc2ocnc2c(F)c1Nc1ccc(I)cc1F ; O=C(NOCC(O)CO)c1cc2ocnc2c(F)c1Nc1ccc(I)cc1F ; O=C(NOCCO)c1cc2ocnc2c(F)c1Nc1ccc(Br)cc1Cl ; O=C(NOCC(O)CO)c1cc2ocnc2c(F)c1Nc1ccc(Br)cc1Cl]

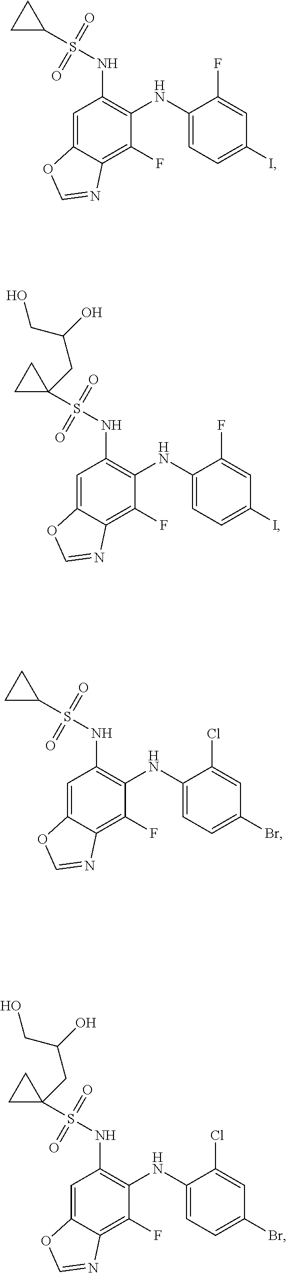 [SMILES: O=S(=O)(Nc1cc2ocnc2c(F)c1Nc1ccc(I)cc1F)C1CC1 ; O=S(=O)(Nc1cc2ocnc2c(F)c1Nc1ccc(I)cc1F)C1(CC(O)CO)CC1 ; O=S(=O)(Nc1cc2ocnc2c(F)c1Nc1ccc(Br)cc1Cl)C1CC1 ; O=S(=O)(Nc1cc2ocnc2c(F)c1Nc1ccc(Br)cc1Cl)C1(CC(O)CO)CC1]

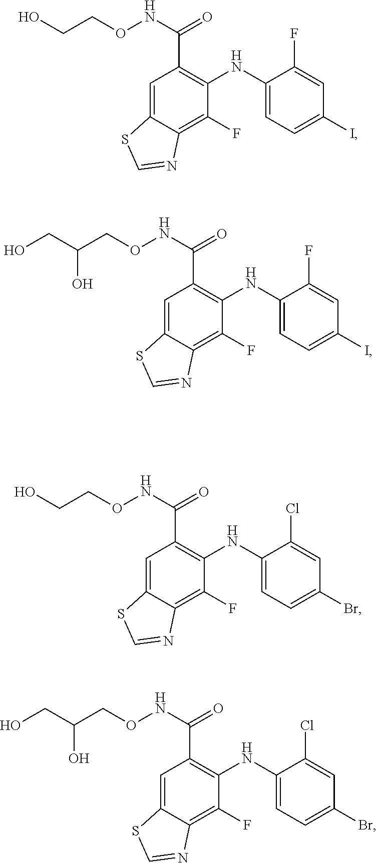 [SMILES: O=C(NOCCO)c1cc2scnc2c(F)c1Nc1ccc(I)cc1F ; O=C(NOCC(O)CO)c1cc2scnc2c(F)c1Nc1ccc(I)cc1F ; O=C(NOCCO)c1cc2scnc2c(F)c1Nc1ccc(Br)cc1Cl ; O=C(NOCC(O)CO)c1cc2scnc2c(F)c1Nc1ccc(Br)cc1Cl]

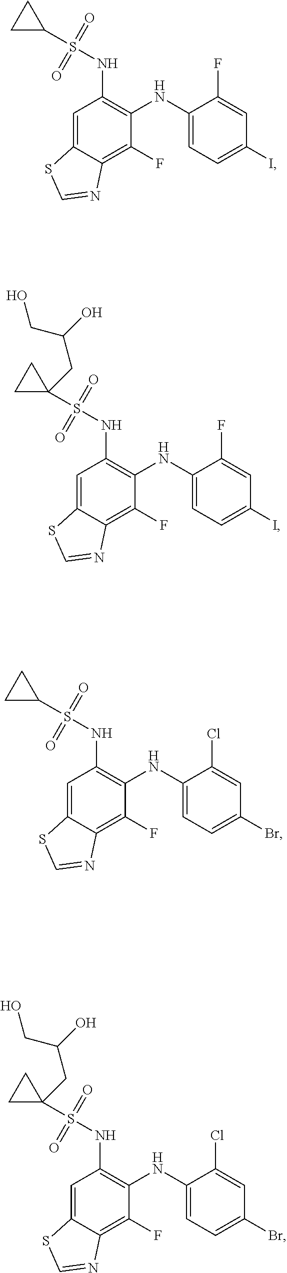 [SMILES: O=S(=O)(Nc1cc2scnc2c(F)c1Nc1ccc(I)cc1F)C1CC1 ; O=S(=O)(Nc1cc2scnc2c(F)c1Nc1ccc(I)cc1F)C1(CC(O)CO)CC1 ; O=S(=O)(Nc1cc2scnc2c(F)c1Nc1ccc(Br)cc1Cl)C1CC1 ; O=S(=O)(Nc1cc2scnc2c(F)c1Nc1ccc(Br)cc1Cl)C1(CC(O)CO)CC1]

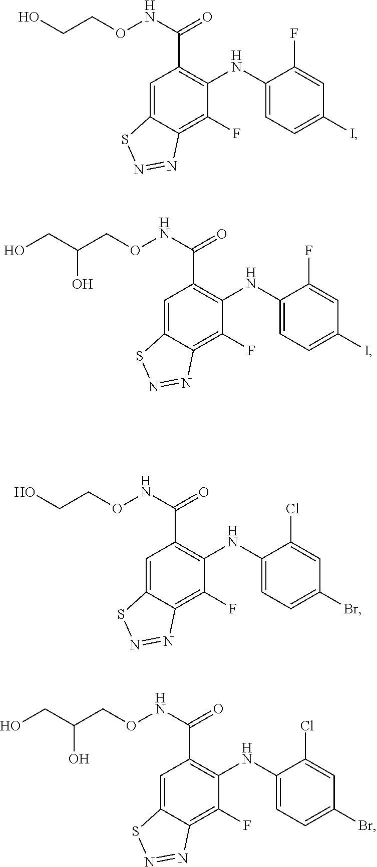 [SMILES: O=C(NOCCO)c1cc2snnc2c(F)c1Nc1ccc(I)cc1F ; O=C(NOCC(O)CO)c1cc2snnc2c(F)c1Nc1ccc(I)cc1F ; O=C(NOCCO)c1cc2snnc2c(F)c1Nc1ccc(Br)cc1Cl ; O=C(NOCC(O)CO)c1cc2snnc2c(F)c1Nc1ccc(Br)cc1Cl]

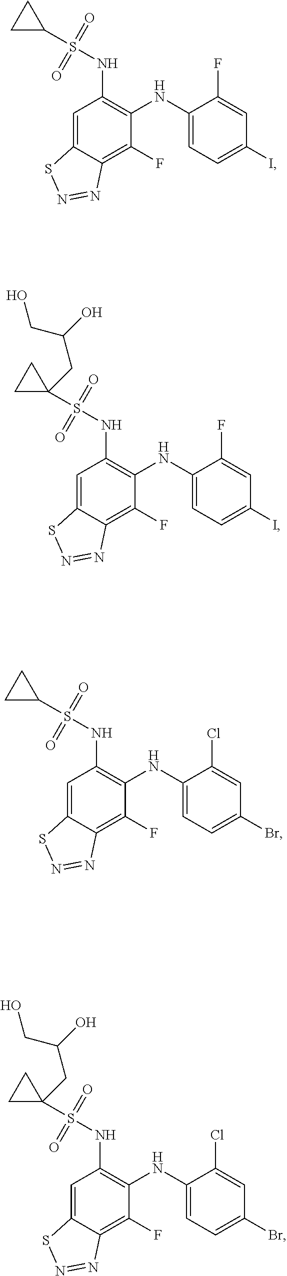 [SMILES: O=S(=O)(Nc1cc2snnc2c(F)c1Nc1ccc(I)cc1F)C1CC1 ; O=S(=O)(Nc1cc2snnc2c(F)c1Nc1ccc(I)cc1F)C1(CC(O)CO)CC1 ; O=S(=O)(Nc1cc2snnc2c(F)c1Nc1ccc(Br)cc1Cl)C1CC1 ; O=S(=O)(Nc1cc2snnc2c(F)c1Nc1ccc(Br)cc1Cl)C1(CC(O)CO)CC1]

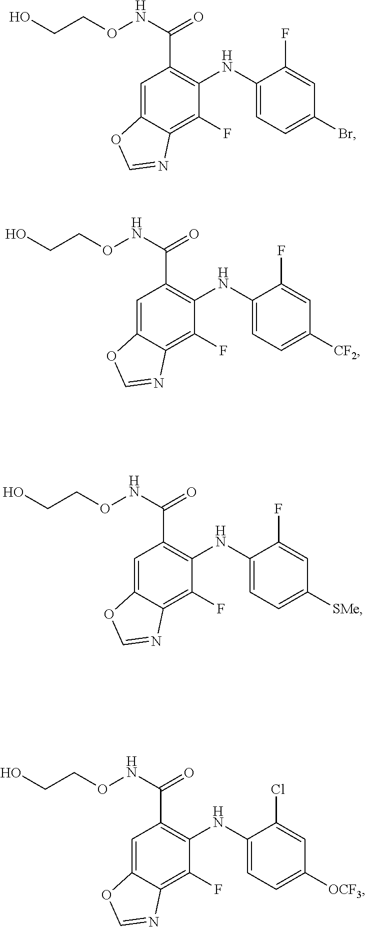

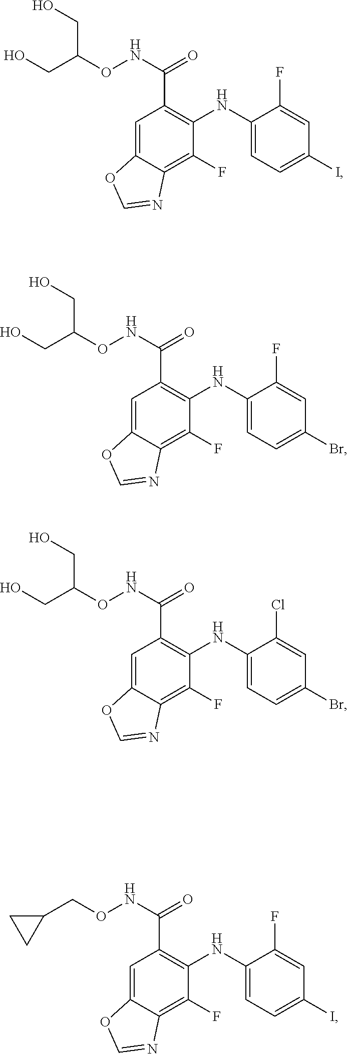 [SMILES: O=C(NOC(CO)CO)c1cc2ocnc2c(F)c1Nc1ccc(I)cc1F ; O=C(NOC(CO)CO)c1cc2ocnc2c(F)c1Nc1ccc(Br)cc1F ; O=C(NOC(CO)CO)c1cc2ocnc2c(F)c1Nc1ccc(Br)cc1Cl ; O=C(NOCC1CC1)c1cc2ocnc2c(F)c1Nc1ccc(I)cc1F]

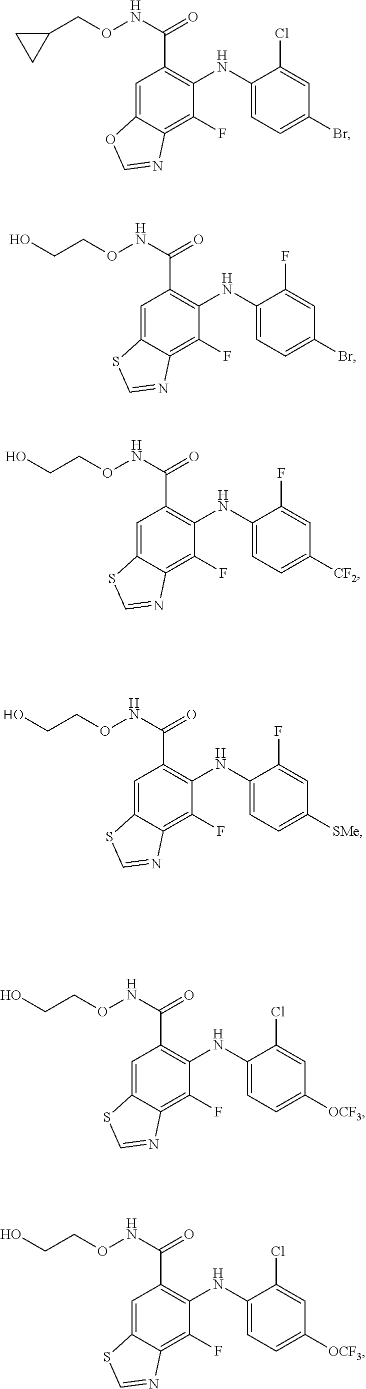

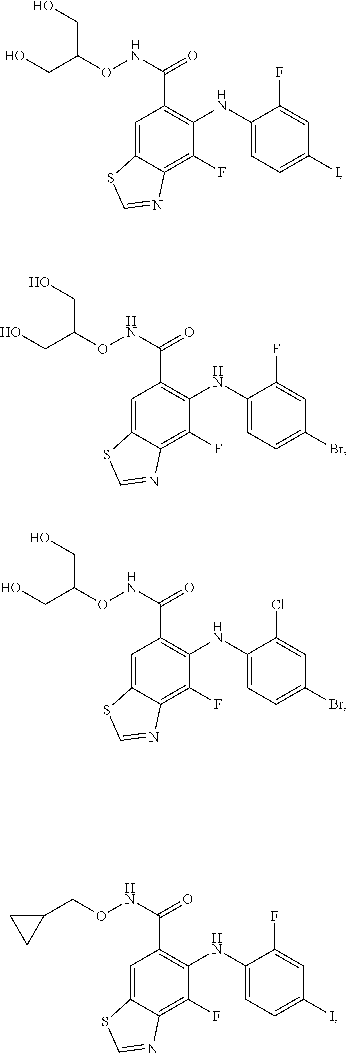 [SMILES: O=C(NOC(CO)CO)c1cc2scnc2c(F)c1Nc1ccc(I)cc1F ; O=C(NOC(CO)CO)c1cc2scnc2c(F)c1Nc1ccc(Br)cc1F ; O=C(NOC(CO)CO)c1cc2scnc2c(F)c1Nc1ccc(Br)cc1Cl ; O=C(NOCC1CC1)c1cc2scnc2c(F)c1Nc1ccc(I)cc1F]

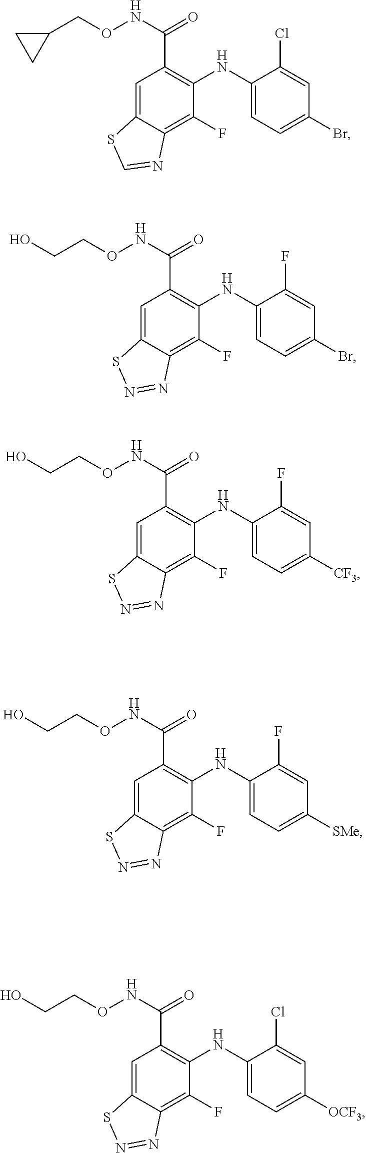 [SMILES: O=C(NOCC1CC1)c1cc2scnc2c(F)c1Nc1ccc(Br)cc1Cl ; O=C(NOCCO)c1cc2snnc2c(F)c1Nc1ccc(Br)cc1F ; O=C(NOCCO)c1cc2snnc2c(F)c1Nc1ccc(C(F)(F)F)cc1F ; CSc1ccc(Nc2c(C(=O)NOCCO)cc3snnc3c2F)c(F)c1 ; O=C(NOCCO)c1cc2snnc2c(F)c1Nc1ccc(OC(F)(F)F)cc1Cl]

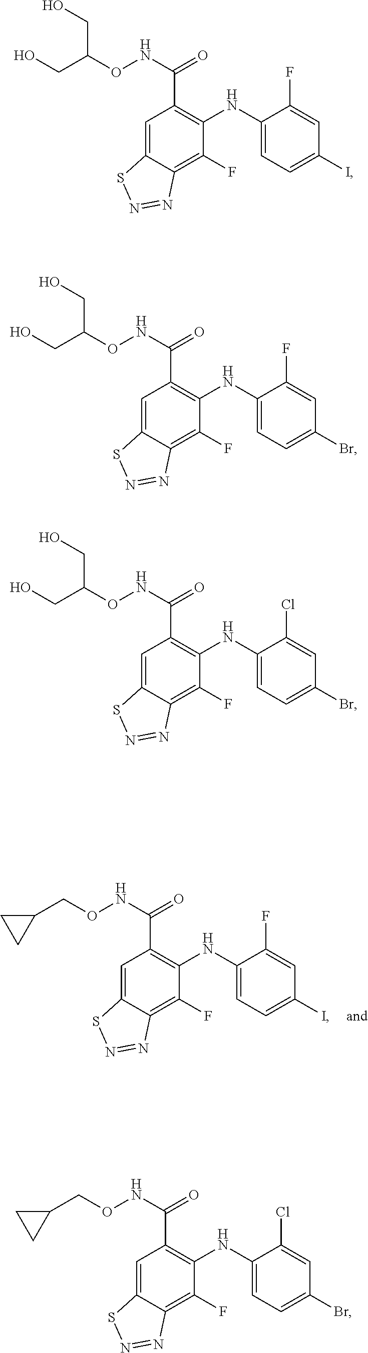 [SMILES: O=C(NOC(CO)CO)c1cc2snnc2c(F)c1Nc1ccc(I)cc1F ; O=C(NOC(CO)CO)c1cc2snnc2c(F)c1Nc1ccc(Br)cc1F ; O=C(NOC(CO)CO)c1cc2snnc2c(F)c1Nc1ccc(Br)cc1Cl ; O=C(NOCC1CC1)c1cc2snnc2c(F)c1Nc1ccc(I)cc1F ; O=C(NOCC1CC1)c1cc2snnc2c(F)c1Nc1ccc(Br)cc1Cl]

and their pharmaceutically acceptable salts. In some embodiments, the MEK inhibitor is CIP-137401. In some embodiments, the MEK inhibitor has a CAS No. 1404099-63-3. In some embodiments, the MEK inhibitor is selumetinib.

In some embodiments, the MEK inhibitor or a salt thereof is administered to a subject at about 5 mg to about 500 mg. In some embodiments, the MEK inhibitor or a salt thereof is administered to a subject at about 10 mg to about 150 mg. In some embodiments, the MEK inhibitor or a salt thereof is administered to a subject at about 10 mg to about 125 mg. In some embodiments, the MEK inhibitor or a salt thereof is administered to a subject at about 10 mg to about 100 mg. In some embodiments, the MEK inhibitor or a salt thereof is administered to a subject at about 25 mg to about 100 mg. In some embodiments, the MEK inhibitor or a salt thereof is administered to a subject at about 50 mg to about 100 mg. In some embodiments, the MEK inhibitor or a salt thereof is administered to a subject at about 5 mg to about 75 mg. In some embodiments, the MEK inhibitor or a salt thereof is administered to a subject at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 105 mg, about 110 mg, about 115 mg, about 120 mg, about 125 mg, about 130 mg, about 135 mg, about 140 mg, about 145 mg, or about 150 mg. In some embodiments, the MEK inhibitor or a salt thereof is administered to a subject at about 15 mg, about 30 mg, about 45 mg, or about 60 mg. In some embodiments, the MEK inhibitor or a salt thereof is administered to a subject at about 15 mg. In some embodiments, the MEK inhibitor or a salt thereof is administered to a subject at about 30 mg. In some embodiments, the MEK inhibitor or a salt thereof is administered to a subject at about 45 mg. In some embodiments, the MEK inhibitor or a salt thereof is administered to a subject at about 60 mg. In some embodiments, the subject is between 12 years old to 18 years old. In some embodiments, the subject is between greater than or equal 12 years old to less than or equal to 18 years. In some embodiments, the subject is an adult. In some embodiments, the subject is greater than or equal to 18 years old.

In some embodiments, a MEK inhibitor or a salt thereof described herein is administered once daily. In some embodiments, the MEK or a salt thereof is administered twice daily. In some embodiments, the MEK or a salt thereof is administered 3 times daily. In some embodiments, the MEK or a salt thereof is administered once weekly. In some embodiments, the MEK or a salt thereof is administered every other day. In some embodiments, the MEK or a salt thereof is administered every 3 days.

In some embodiments, the MEK inhibitor or a salt thereof is administered to a subject at 10 mg to 150 mg. In some embodiments, the MEK inhibitor or a salt thereof is administered to a subject at 10 mg to 125 mg. In some embodiments, the MEK inhibitor or a salt thereof is administered to a subject at 10 mg to 100 mg. In some embodiments, the MEK inhibitor or a salt thereof is administered to a subject at 25 mg to 100 mg. In some embodiments, the MEK inhibitor or a salt thereof is administered to a subject at 50 mg to 100 mg. In some embodiments, the MEK inhibitor or a salt thereof is administered to a subject at 5 mg to 75 mg. In some embodiments, the MEK inhibitor or a salt thereof is administered to a subject at 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 100 mg, 105 mg, 110 mg, 115 mg, 120 mg, 125 mg, 130 mg, 135 mg, 140 mg, 145 mg, or 150 mg. In some embodiments, the MEK inhibitor or a salt thereof is administered to a subject at 15 mg, 30 mg, 45 mg, or 60 mg. In some embodiments, the MEK inhibitor or a salt thereof is administered to a subject at 15 mg. In some embodiments, the MEK inhibitor or a salt thereof is administered to a subject at 30 mg. In some embodiments, the MEK inhibitor or a salt thereof is administered to a subject at 45 mg. In some embodiments, the MEK inhibitor or a salt thereof is administered to a subject at 60 mg. In some embodiments, the subject is between 12 years old to 18 years old. In some embodiments, the subject is between greater than or equal 12 years old to less than or equal to 18 years. In some embodiments, the subject is an adult. In some embodiments, the subject is greater than or equal to 18 years old.

In some embodiments, the MEK inhibitor or a salt thereof is administered to a subject at about 10 mg to about 150 mg, daily. In some embodiments, the MEK inhibitor or a salt thereof is administered to a subject at about 10 mg to about 125 mg, daily. In some embodiments, the MEK inhibitor or a salt thereof is administered to a subject at about 10 mg to about 100 mg, daily. In some embodiments, the MEK inhibitor or a salt thereof is administered to a subject at about 25 mg to about 100 mg, daily. In some embodiments, the MEK inhibitor or a salt thereof is administered to a subject at about 50 mg to about 100 mg, daily. In some embodiments, the MEK inhibitor or a salt thereof is administered to a subject at about 5 mg to about 75 mg, daily. In some embodiments, the MEK inhibitor or a salt thereof is administered to a subject at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 105 mg, about 110 mg, about 115 mg, about 120 mg, about 125 mg, about 130 mg, about 135 mg, about 140 mg, about 145 mg, or about 150 mg, daily. In some embodiments, the MEK inhibitor or a salt thereof is administered to a subject at about 15 mg, about 30 mg, about 45 mg, or about 60 mg, daily. In some embodiments, the MEK inhibitor or a salt thereof is administered to a subject at about 15 mg, daily. In some embodiments, the MEK inhibitor or a salt thereof is administered to a subject at about 30 mg, daily. In some embodiments, the MEK inhibitor or a salt thereof is administered to a subject at about 45 mg, daily. In some embodiments, the MEK inhibitor or a salt thereof is administered to a subject at about 60 mg, daily. In some embodiments, the MEK inhibitor or a salt thereof is administered once daily. In some embodiments, the subject is between 12 years old to 18 years old. In some embodiments, the subject is between greater than or equal 12 years old to less than or equal to 18 years. In some embodiments, the subject is an adult. In some embodiments, the subject is greater than or equal to 18 years old.

In some embodiments, the MEK inhibitor or a salt thereof is administered to a subject at about 10 mg to about 150 mg, twice daily. In some embodiments, the MEK inhibitor or a salt thereof is administered to a subject at about 10 mg to about 125 mg, twice daily. In some embodiments, the MEK inhibitor or a salt thereof is administered to a subject at about 10 mg to about 100 mg, twice daily. In some embodiments, the MEK inhibitor or a salt thereof is administered to a subject at about 25 mg to about 100 mg, twice daily. In some embodiments, the MEK inhibitor or a salt thereof is administered to a subject at about 50 mg to about 100 mg, twice daily. In some embodiments, the MEK inhibitor or a salt thereof is administered to a subject at about 5 mg to about 75 mg, twice daily. In some embodiments, the MEK inhibitor or a salt thereof is administered to a subject at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 105 mg, about 110 mg, about 115 mg, about 120 mg, about 125 mg, about 130 mg, about 135 mg, about 140 mg, about 145 mg, or about 150 mg, twice daily. In some embodiments, the MEK inhibitor or a salt thereof is administered to a subject at about 15 mg, about 30 mg, about 45 mg, or about 60 mg, twice daily. In some embodiments, the MEK inhibitor or a salt thereof is administered to a subject at about 15 mg, twice daily. In some embodiments, the MEK inhibitor or a salt thereof is administered to a subject at about 30 mg, twice daily. In some embodiments, the MEK inhibitor or a salt thereof is administered to a subject at about 45 mg, twice daily. In some embodiments, the MEK inhibitor or a salt thereof is administered to a subject at about 60 mg, twice daily. In some embodiments, the subject is between 12 years old to 18 years old. In some embodiments, the subject is between greater than or equal 12 years old to less than or equal to 18 years. In some embodiments, the subject is an adult. In some embodiments, the subject is greater than or equal to 18 years old.

In some embodiments, the MEK inhibitor or a salt thereof is Pimasertib or a salt thereof. In some embodiments, the MEK inhibitor or a salt thereof is Pimasertib hydrocholoride. In some embodiments, the MEK inhibitor is Pimasertib. In some embodiments, the dosing described herein for a MEK inhibitor or a salt thereof is based on the weight of the MEK inhibitor. In some embodiments, the dosing described herein for a MEK inhibitor or a salt thereof corresponds to the weight of the free base form of the MEK inhibitor. For example, in some embodiments, the dosing of the pimasertib or a salt thereof describes the weight of the pimasertib in such dosing. In some embodiments, the dosing described herein for a MEK inhibitor or a salt thereof corresponds to the weight of the salt of the MEK inhibitor.

In some embodiments, Pimasertib (e.g., as a salt of pimasertib or pimasertib free base) is administered to a subject at about 10 mg to about 150 mg. In some embodiments, Pimasertib is administered to a subject at about 10 mg to about 125 mg. In some embodiments, Pimasertib is administered to a subject at about 10 mg to about 100 mg. In some embodiments, Pimasertib is administered to a subject at about 25 mg to about 100 mg. In some embodiments, Pimasertib is administered to a subject at about 50 mg to about 100 mg. In some embodiments, Pimasertib is administered to a subject at about 5 mg to about 75 mg. In some embodiments, Pimasertib is administered to a subject at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 105 mg, about 110 mg, about 115 mg, about 120 mg, about 125 mg, about 130 mg, about 135 mg, about 140 mg, about 145 mg, or about 150 mg. In some embodiments, Pimasertib is administered to a subject at about 15 mg, about 30 mg, about 45 mg, or about 60 mg. In some embodiments, Pimasertib is administered to a subject at about 15 mg. In some embodiments, Pimasertib is administered to a subject at about 30 mg. In some embodiments, Pimasertib is administered to a subject at about 45 mg. In some embodiments, Pimasertib is administered to a subject at about 60 mg. In some embodiments, the subject is 12 years old to 18 years old. In some embodiments, the subject is between greater than or equal 12 years old to less than or equal to 18 years. In some embodiments, the subject is an adult. In some embodiments, the subject is greater than or equal to 18 years old. In some embodiments, Pimasertib is administered in the form of a salt of pimasertib such as Pimasertib HCl.

In some embodiments, Pimasertib (e.g., as a salt of pimasertib or pimasertib free base) is administered to a subject at 10 mg to 150 mg. In some embodiments, Pimasertib is administered to a subject at 10 mg to 125 mg. In some embodiments, Pimasertib is administered to a subject at 10 mg to 100 mg. In some embodiments, Pimasertib is administered to a subject at 25 mg to 100 mg. In some embodiments, Pimasertib is administered to a subject at 50 mg to 100 mg. In some embodiments, Pimasertib is administered to a subject at 5 mg to 75 mg. In some embodiments, Pimasertib is administered to a subject at 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 100 mg, 105 mg, 110 mg, 115 mg, 120 mg, 125 mg, 130 mg, 135 mg, 140 mg, 145 mg, or 150 mg. In some embodiments, Pimasertib is administered to a subject at 15 mg, 30 mg, 45 mg, or 60 mg. In some embodiments, Pimasertib is administered to a subject at 15 mg. In some embodiments, Pimasertib is administered to a subject at 30 mg. In some embodiments, Pimasertib is administered to a subject at 45 mg. In some embodiments, Pimasertib is administered to a subject at 60 mg. In some embodiments, the subject is 12 years old to 18 years old. In some embodiments, the subject is between greater than or equal 12 years old to less than or equal to 18 years. In some embodiments, the subject is an adult. In some embodiments, the subject is greater than or equal to 18 years old. In some embodiments, Pimasertib is administered in the form of a salt of pimasertib such as Pimasertib HCl.

In some embodiments, Pimasertib (e.g., as a salt of pimasertib or pimasertib free base) is administered to a subject at about 10 mg to about 150 mg, daily. In some embodiments, Pimasertib is administered to a subject at about 10 mg to about 125 mg, daily. In some embodiments, Pimasertib is administered to a subject at about 10 mg to about 100 mg, daily. In some embodiments, Pimasertib is administered to a subject at about 25 mg to about 100 mg, daily. In some embodiments, Pimasertib is administered to a subject at about 50 mg to about 100 mg, daily. In some embodiments, Pimasertib is administered to a subject at about 5 mg to about 75 mg, daily. In some embodiments, Pimasertib is administered to a subject at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 105 mg, about 110 mg, about 115 mg, about 120 mg, about 125 mg, about 130 mg, about 135 mg, about 140 mg, about 145 mg, or about 150 mg, daily. In some embodiments, Pimasertib is administered to a subject at about 15 mg, about 30 mg, about 45 mg, or about 60 mg, daily. In some embodiments, Pimasertib is administered to a subject at about 15 mg, daily. In some embodiments, Pimasertib is administered to a subject at about 30 mg, daily. In some embodiments, Pimasertib is administered to a subject at about 45 mg, daily. In some embodiments, Pimasertib is administered to a subject at about 60 mg, daily. In some embodiments, pimasertib is administered once daily In some embodiments, the subject is between 12 years old to 18 years old. In some embodiments, the subject is between greater than or equal 12 years old to less than or equal to 18 years. In some embodiments, the subject is an adult. In some embodiments, the subject is greater than or equal to 18 years old. In some embodiments, Pimasertib is administered in the form of a salt of pimasertib such as Pimasertib HCl.

In some embodiments, Pimasertib (e.g., as a salt of pimasertib or pimasertib free base) is administered to a subject at about 10 mg to about 150 mg, twice daily. In some embodiments, Pimasertib is administered to a subject at about 10 mg to about 125 mg, twice daily. In some embodiments, Pimasertib is administered to a subject at about 10 mg to about 100 mg, twice daily. In some embodiments, Pimasertib is administered to a subject at about 25 mg to about 100 mg, twice daily. In some embodiments, Pimasertib is administered to a subject at about 50 mg to about 100 mg, twice daily. In some embodiments, Pimasertib is administered to a subject at about 5 mg to about 75 mg, twice daily. In some embodiments, Pimasertib is administered to a subject at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 105 mg, about 110 mg, about 115 mg, about 120 mg, about 125 mg, about 130 mg, about 135 mg, about 140 mg, about 145 mg, or about 150 mg, twice daily. In some embodiments, Pimasertib is administered to a subject at about 15 mg, about 30 mg, about 45 mg, or about 60 mg, twice daily. In some embodiments, Pimasertib is administered to a subject at about 15 mg, twice daily. In some embodiments, Pimasertib is administered to a subject at about 30 mg, twice daily. In some embodiments, Pimasertib is administered to a subject at about 45 mg, twice daily. In some embodiments, Pimasertib is administered to a subject at about 60 mg, twice daily. In some embodiments, the subject is 12 years old to 18 years old. In some embodiments, the subject is between greater than or equal 12 years old to less than or equal to 18 years. In some embodiments, the subject is an adult. In some embodiments, the subject is greater than or equal to 18 years old. In some embodiments, Pimasertib is administered in the form of a salt of pimasertib such as Pimasertib HCl.

In some embodiments, Pimasertib (e.g., as a salt of pimasertib or pimasertib free base) is administered to a subject at about 10 mg to about 150 mg, every other day. In some embodiments, Pimasertib is administered to a subject at about 10 mg to about 125 mg, every other day. In some embodiments, Pimasertib is administered to a subject at about 10 mg to about 100 mg, every other day. In some embodiments, Pimasertib is administered to a subject at about 25 mg to about 100 mg, every other day. In some embodiments, Pimasertib is administered to a subject at about 50 mg to about 100 mg, every other day. In some embodiments, Pimasertib is administered to a subject at about 5 mg to about 75 mg, every other day. In some embodiments, Pimasertib is administered to a subject at about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 105 mg, about 110 mg, about 115 mg, about 120 mg, about 125 mg, about 130 mg, about 135 mg, about 140 mg, about 145 mg, or about 150 mg, every other day. In some embodiments, Pimasertib is administered to a subject at about 15 mg, about 30 mg, about 45 mg, or about 60 mg, every other day. In some embodiments, Pimasertib is administered to a subject at about 15 mg, every other day. In some embodiments, Pimasertib is administered to a subject at about 30 mg, every other day. In some embodiments, Pimasertib is administered to a subject at about 45 mg, every other day. In some embodiments, Pimasertib is administered to a subject at about 60 mg, every other day. In some embodiments, the subject is between 12 years old to 18 years old. In some embodiments, the subject is between greater than or equal 12 years old to less than or equal to 18 years. In some embodiments, the subject is an adult. In some embodiments, the subject is greater than or equal to 18 years old. In some embodiments, Pimasertib is administered in the form of a salt of pimasertib such as Pimasertib HCl.

In some embodiments, selumetinib (e.g., as a salt of selumetinib or selumetinib free base) is administered to a subject at about 2 mg to about 15 mg. In some embodiments, selumetinib is administered to a subject at about 5 mg to about 15 mg. In some embodiments, selumetinib is administered to a subject at about 10 mg to about 15 mg. In some embodiments, selumetinib is administered to a subject at about 2 mg to about 8 mg. In some embodiments, selumetinib is administered to a subject at about 2 mg, about 3 mg, about 4 mg about 5 mg, about 6 mg, about 7 mg, about 8 mg, about 9 mg, about 10 mg, about 11 mg, about 12 mg, about 13 mg, about 14 mg, or about 15 mg. In some embodiments, selumetinib is administered to a subject at about 2 mg, about 3 mg, about 4 mg about 5 mg, or about 6 mg. In some embodiments, selumetinib is administered to a subject at about 7 mg, about 8 mg, about 9 mg, about 10 mg, or about 11 mg. In some embodiments, selumetinib is administered to a subject at about 12 mg, about 13 mg, about 14 mg, or about 15 mg. In some embodiments, the subject is between 12 years old to 18 years old. In some embodiments, the subject is between greater than or equal 12 years old to less than or equal to 18 years. In some embodiments, the subject is an adult. In some embodiments, the subject is greater than or equal to 18 years old.

In some embodiments, selumetinib (e.g., as a salt of selumetinib or selumetinib free base) is administered to a subject at about 2 mg to about 15 mg, daily. In some embodiments, selumetinib is administered to a subject at about 5 mg to about 15 mg, daily. In some embodiments, selumetinib is administered to a subject at about 10 mg to about 15 mg, daily. In some embodiments, selumetinib is administered to a subject at about 2 mg to about 8 mg, daily. In some embodiments, selumetinib is administered to a subject at about 2 mg, about 3 mg, about 4 mg about 5 mg, about 6 mg, about 7 mg, about 8 mg, about 9 mg, about 10 mg, about 11 mg, about 12 mg, about 13 mg, about 14 mg, or about 15 mg, daily. In some embodiments, selumetinib is administered to a subject at about 2 mg, about 3 mg, about 4 mg about 5 mg, or about 6 mg, daily. In some embodiments, selumetinib is administered to a subject at about 7 mg, about 8 mg, about 9 mg, about 10 mg, or about 11 mg, daily. In some embodiments, selumetinib is administered to a subject at about 12 mg, about 13 mg, about 14 mg, or about 15 mg, daily. In some embodiments, the subject is between 12 years old to 18 years old. In some embodiments, the subject is between greater than or equal 12 years old to less than or equal to 18 years. In some embodiments, the subject is an adult. In some embodiments, the subject is greater than or equal to 18 years old.

In some embodiments, selumetinib (e.g., as a salt of selumetinib or selumetinib free base) is administered to a subject at about 2 mg to about 15 mg, twice daily. In some embodiments, selumetinib is administered to a subject at about 5 mg to about 15 mg, twice daily. In some embodiments, selumetinib is administered to a subject at about 10 mg to about 15 mg, twice daily. In some embodiments, selumetinib is administered to a subject at about 2 mg to about 8 mg, twice daily. In some embodiments, selumetinib is administered to a subject at about 2 mg, about 3 mg, about 4 mg about 5 mg, about 6 mg, about 7 mg, about 8 mg, about 9 mg, about 10 mg, about 11 mg, about 12 mg, about 13 mg, about 14 mg, or about 15 mg, twice daily. In some embodiments, selumetinib is administered to a subject at about 2 mg, about 3 mg, about 4 mg about 5 mg, or about 6 mg, twice daily. In some embodiments, selumetinib is administered to a subject at about 7 mg, about 8 mg, about 9 mg, about 10 mg, or about 11 mg, twice daily. In some embodiments, selumetinib is administered to a subject at about 12 mg, about 13 mg, about 14 mg, or about 15 mg, twice daily. In some embodiments, the subject is between 12 years old to 18 years old. In some embodiments, the subject is between greater than or equal 12 years old to less than or equal to 18 years. In some embodiments, the subject is an adult. In some embodiments, the subject is greater than or equal to 18 years old.

### C. Mutations and Indications

The methods presented herein may be used to treat a high unmet medical need cancer. In some embodiments, the method is used to treat a genetically defined subset of cancer. In some embodiments, the cancer has one or more of the following mutations: RAS positive mutation, RAF positive mutation, MEK positive mutation, and ERK positive mutation. In some embodiments, the cancer has an NRAS mutation, a KRAS mutation, or HRAS mutation. In some embodiments, the cancer has a BRAF mutation, a BRAF fusion, or a CRAF fusion. In some embodiments, the BRAF mutation is a non-V600 BRAF mutation. In some embodiments, the BRAF mutation is V600 BRAF mutation. In some embodiments, the cancer has a Class I BRAF mutation, a Class II BRAF mutation, or a Class III BRAF mutation. In some embodiments, the subject has a Class I BRAF mutation, a Class II BRAF mutation, or Class III BRAF mutation. In some embodiments, the cancer has a Class I BRAF mutation. In some embodiments, the cancer has a Class II BRAF mutation. In some embodiments, the cancer has a Class III BRAF mutation. In some embodiments, the subject has a Class I BRAF mutation or a Class II BRAF mutation. In some embodiments, the subject lacks V600E mutation, V600K mutation, or both. In some embodiments, the subject has a non-V600 BRAF mutation. In some embodiments, the methods disclosed herein have anti-proliferative activity in a subject. In some embodiments, the cancer has a genomic alteration resulting in a dependency on signaling through the MAPK pathway.

In some embodiments, the cancer is a recurrent, progressive, or refractory solid tumor with mitogen-activated protein kinase (MAPK) pathway aberration. In some embodiments, the cancer is recurrent with mitogen-activated protein kinase (MAPK) pathway aberration. In some embodiments, the cancer is progressive with mitogen-activated protein kinase (MAPK) pathway aberration. In some embodiments, the cancer is refractory with mitogen-activated protein kinase (MAPK) pathway aberration. In some embodiments, the cancer is a recurrent or progressive solid tumor with aberrations in the key proteins of the mitogen-activated protein kinase (MAPK) pathway, such as tumors that harbor RAS or RAF alterations. In some embodiments, the cancer is a recurrent or progressive solid tumor with aberrations in the key proteins of the mitogen-activated protein kinase (MAPK) pathway, such as tumors that harbor a BRAF fusion or a CRAF fusion.

In some embodiments, the cancer has a mitogen-activated protein kinase (MAPK) aberration. In some embodiments, the cancer has a mitogen-activated protein kinase (MAPK) aberration selected from a mutation or gene fusion. In some embodiments, the MAPK aberration is selected from a RAS positive mutation, a RAF positive mutation, a MEK positive mutation, a ERK positive mutation and a gene fusion. In some embodiments, the cancer has a MAPK aberration selected from a NRAS mutation, a KRAS mutation, or a HRAS mutation. In some embodiments, the cancer has a MAPK aberration selected from a BRAF mutation, a BRAF fusion, and a CRAF fusion.

In some embodiments, the subject is identified having one or more BRAF fusions. In some embodiments, the subject is identified having one or more of the following fusions: KIAA1549:BRAF, STARD3NL:BRAF, BCASI:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, AGK:BRAF, AGAP3:BRAF, TNS3:BRAF, TARDBP:BRAF, ARMC10:BRAF, CUL1:BRAF, TRIM24:BRAF, AKAP9:BRAF, FKBP15:BRAF, SKAP2:BRAF, ZKSCAN1:BRAF, KLHL7:BRAF, SEPT3:BRAF, SRGAP3:RAF1, QK1:RAF1, FYCO:RAF1, ATG7:RAF1, and NFIA:RAF1. In some embodiments, the subject is identified having KIAA1549:BRAF fusion. In some embodiments, the methods disclosed herein are BRAF fusions.

In some embodiments, the methods disclosed herein may be used to modulate RAF monomers or dimers. In some embodiments, RAF monomers are modulated. In some embodiments, RAF dimers are modulated. In some embodiments, the modulation disclosed herein is inhibition.

In some embodiments, the cancer is B-Raf mutation-positive cancer (i.e., the cancer has one or more B-Raf mutations). In some embodiments, the B-Raf mutation is in exon 11 or 15. In some embodiments, the B- Raf mutation is in codon 466, 469, 594, 600, or 601. In some embodiments, the B-Raf mutation is in codon 600. In some embodiments, the B-Raf mutation includes but is not limited to a V600E, V600D or V600K mutation. In some embodiments, the B-Raf mutation is V600E. In some embodiments, the B-Raf mutation is V600D. In some embodiments, the B-Raf mutation is V600K. In some embodiments, the B-Raf mutation is V600E+T5291. In some embodiments, the B-Raf mutation is V600E+G468A. “V600E mutation” means substitution of glutamic acid for valine at the amino acid position of 600. T529I is a threonine to isoleucine B-Raf gatekeeper mutation and G468A is a B-Raf secondary mutation at G1403C in exon 11. “V600K mutation” means substitution of lysine for valine at the amino acid position of 600. “V600D mutation” means substitution of aspartic acid for valine at the amino acid position of 600. The V600K mutation results in an amino acid substitution at position 600 in B-Raf, from a valine (V) to a lysine (K) The V600K mutation results in an amino acid substitution at position 600 in B-Raf, from a valine (V) to a lysine (K)).

In some embodiments, the cancer is a non-V600 B-Raf mutation positive cancer (i.e., the cancer has one or more B-Raf mutations and the one or more mutations is not B-Raf V600). In some embodiments, the B-Raf mutation is in exon 11 or 15. In some embodiments, the B-Raf mutation is in codon 466, 469, 594, or 601. In one aspect, one or more non-V600E mutation is G466A, G466V, N581S,D594H, R146W, L613F, D565_splice, S394*, P367R, G469A, G469V, G469*, G466V, G464V, G397S, SI 131, A762E, G469L, D594N, G596S, G596R, D594N, D594H, K601E, K601N, L597Q, L597V, G469R, D594G, or G327_splice. In one aspect, one or more non-V600E mutations are G469R, R95T, A621_splice, V639I, Q609H, G464V, or G466V. The asterisk “*” means a stop codon.

In some embodiments, a cancer described herein has a V600 BRAF mutation. In some embodiments, a cancer described herein has a gene mutation or fusion described in Tables 1-7.

In some embodiments, the cancer is identified as having a non V600 BRAF mutation is selected from: V600E, G469A, G464V, G466V, K601E, G469R, and L597R. In some embodiments, the non V600 BRAF mutation is selected from: V600E, G464A, G464V, K601E, and G469R. In some embodiments, the non V600 BRAF mutation is selected from: G464V, K601E, G469A, and G466V.

In some embodiments, the cancer is K-Ras mutation-positive cancer (i.e., the cancer has one or more K-Ras mutations). In some embodiments, the K-Ras mutation is in exon 2. In some embodiments, the K-Ras mutation is in codon 12 or 13. In some embodiments, the cancer is identified as having a RAS mutation. In some embodiments, the RAS mutation is a KRAS mutation. In some embodiments, the KRAS mutation is selected from: KRAS G12C, KRAS G12V, KRAS G12D, KRAS Q61K, KRAS Q61H, KRAS G13D, and KRAS G12S. In some embodiments, the KRAS mutation is selected from: KRAS G12C, KRAS G12D, KRAS G13D, and KRAS G12S.

In some embodiments, the cancer is N-Ras mutation-positive cancer (i.e., the cancer has one or more N-Ras mutations). In some embodiments, the N-Ras mutation is in exon 2, 3, or 4. In some embodiments, the N-Ras mutation is in exon 2. In some embodiments, the N-Ras mutation is in exon 3. In some embodiments, the N-Ras mutation is in exon 4. In some embodiments, the N-Ras mutation is Q61R, Q61K, Q61L, Q61H, or Q61P. In some embodiments, the N-Ras mutation is Q61R mutation.

The present disclosure provides a method of treating a subject suffering from cancer. In some embodiments, the cancer is selected from lung cancer, colorectal cancer, pancreatic cancer, skin cancer, glioma, nonglioma brain cancer, bone sarcomas, gastrointestinal cancer, breast cancer, thyroid cancer, acute lymphocytic leukemia (ALL), acute myeloid leukemia (AML), and multiple myeloma (MM). In some embodiments, lung cancer includes non-small cell lung cancer (NSCLC) and small cell lung cancer (SCLC). In some embodiments, the cancer is not NSCLC. In some embodiments, the cancer is a urothelial tumor. In some embodiments, the cancer is a low-grade glioma (LGG). In some embodiments, the cancer is a pediatric low-grad glioma (PLGG). In some embodiments, the LGG is newly diagnosed. In some embodiments, the cancer is a pediatric brain tumor. In some embodiments, the cancer is neuroblastoma. In some embodiments, the cancer is a urothelial tumor with focal amplification of the RAF1 kinase gene. In some embodiments, the cancer is a RAF1 amplified tumor. In some embodiments, the cancer is a RAF1-amplified tumor that exhibits activation of the MAPK signaling pathway and exhibits a luminal gene expression pattern. In some embodiments, the cancer an advanced solid tumor.

In some embodiments, the cancer is a recurrent, progressive, or refractory. In some embodiments, the cancer is recurrent. In some embodiments, the cancer is progressive. In some embodiments, the cancer is refractory.

In some embodiments, a cancer described herein is newly diagnosed. In some embodiments, a cancer described herein has not received any prior cancer treatment. Accordingly, in some embodiments, the methods of treatment described herein can be used as a front-line therapy.

In some embodiments, the cancer is a hematological malignancy. In some embodiments, the hematological malignancy is selected from acute myelogenous leukemia (AML), chronic myelogenous leukemia (CML), chronic lymphoblastic leukemia (CLL), and myelodysplasia syndrome. In some embodiments, the hematogical malignancy is selected from acute myelogenous leukemia (AML) and chronic lymphocytic leukemia (CLL).

In some embodiments, the cancer is selected from thyroid cancer, ovarian cancer, melanoma, acute myelogenous leukemia (AML), and colon cancer. In some embodiments, the cancer is melanoma or colon cancer.

In some embodiments, the cancer is selected from skin cancer and gastrointestinal cancer. In some embodiments, the cancer is skin cancer. In some embodiments, the skin cancer is melanoma. In some embodiments, the melanoma is B-Raf-mutated melanoma. In some embodiments, the melanoma is N-Ras-mutated melanoma. In some embodiments, the cancer is gastrointestinal cancer. As used herein, “gastrointestinal cancer” includes cancer of the esophagus, stomach (also known as gastric cancer), biliary system, pancreas, small intestine, large intestine, rectum and anus). In some embodiments, the gastrointestinal cancer is adenocarcinoma of the esophagus, adenocarcinoma of the gastroesophageal junction or adenocarcinoma of the stomach. In some embodiments, the gastrointestinal cancer is stomach cancer.

In some embodiments, the cancer is a lung cancer, colorectal cancer or pancreatic cancer. In some embodiments, the cancer is lung cancer. In some embodiments, the cancer is non-small cell lung cancer (NSCLC). In some embodiments, the cancer is squamous NSCLC. In some embodiments, the cancer is non-squamous NSCLC.

In some embodiments, the cancer is colon cancer. Colon cancer is also known as colorectal (CRC), bowel, or rectum cancer.

In some embodiments, the cancer is a central nervous system cancer. In some embodiments, the central nervous system cancer is brain cancer. In some embodiments, thyroid cancer is thyroid carcinoma. In some embodiments, genitourinary tract cancer is bladder cancer.

In some embodiments, the cancer is a solid tumor. In some embodiments, the cancer is an advanced solid tumor. In some embodiments, the cancer is a non-small cell lung cancer.

In some embodiments, the cancer is a recurrent cancer. In some embodiments, a subject described herein has received at least one prior therapy that is considered standard of care treatment prior to the administration of Compound A or a pharmaceutically acceptable salt thereof, or the MEK inhibitor. In some embodiments, the prior therapy is a systemic therapy. In some embodiments, the prior therapy is chemotherapy therapy, hormone therapy, immunotherapy, or radiation therapy. In some embodiments, the methods disclosed herein may target MAPK signaling. In some embodiments, the methods disclosed herein may have anti-tumor activity against solid tumors.

### Methods of Identifying

Provided herein is a method of treating cancer, comprising administering an amount of Compound A or a pharmaceutically acceptable salt thereof, and a MEK inhibitor (e.g., pimasertib). In some embodiments, provided herein is a method of treating cancer, comprising administering an amount of Compound A or a pharmaceutically acceptable salt thereof, and a MEK inhibitor (e.g., pimasertib), wherein a total amount of the Compound A or a pharmaceutically acceptable salt thereof and the MEK inhibitor is therapeutically effective in treating the cancer. The present disclosure further provides identifying a subject suffering from cancer. In some embodiments, the methods provided herein provide identifying a subject suffering from cancer, the cancer having one or more of: a RAF alteration, a RAS mutation, an NF-1 mutation or a genomic alteration that results in a dependence on signaling through the MAPK pathway. In some embodiments, the identifying a subject occurs before administering to the subject a RAF inhibitor and MEK inhibitor. In some embodiments, the method of treating a subject suffering from cancer comprises:
(a) identifying a subject suffering from cancer, wherein the cancer has one or more of: a RAF alteration, a RAS mutation, an NF-1 mutation or a genomic alteration that results in a dependence on signaling through the MAPK pathway; and
(b) administering to the subject:
   (i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof; and
   (ii) a MEK inhibitor as provided herein;
      wherein a total amount of the Compound A or a pharmaceutically acceptable salt thereof and the MEK inhibitor is therapeutically effective in treating the cancer. In some embodiments, the MEK inhibitor is an MEK inhibitor as described herein. In some embodiments, the method provides a synergistic effect when administered. The methods of identifying described herein can be combined with any other aspect or embodiment as disclosed herein. In some embodiments, the identifying comprises genomic testing (e.g., mutational testing). In some embodiments, the genetic testing is conducted on a cancer sample of a subject. In some embodiments, the cancer sample of the subject has been subjected to BRAF, KRAS, CRAF, HRAS, NF-1 and/or NRAS mutational testing prior to the administering of Compound A or a pharmaceutically acceptable salt thereof or the MEK inhibitor. In some embodiments, the cancer sample of the subject has been subjected to genomic testing prior to the administering of Compound A or a pharmaceutically acceptable salt thereof or the MEK inhibitor, wherein the genomic testing demonstrates that genomic alteration creates a dependence on MPAK signaling. In some embodiments, the subject is diagnosed with histologically confirmed non-hematologic tumor. In some embodiments, the subject is diagnosed with histologically confirmed hematologic tumor.

In some embodiments, the identifying step comprises identifying the subject with one or more cancer mutations or gene fusion described herein. In some embodiments, the cancer has one or more of the following mutations: RAS positive mutation, RAF positive mutation, MEK positive mutation, ERK positive mutation or any combination thereof. In some embodiments, the identifying comprises identifying a cancer mutation as disclosed herein. In some embodiments, the cancer has a RAS mutation. In some embodiments, the RAS mutation is an HRAS mutation, a KRAS, or an NRAS mutation. In some embodiments, the cancer has a mutation in NF-1 resulting in NF-1 loss-of function.

In some embodiments, the identifying step comprises identifying the subject with a RAF alteration. In some embodiments, the RAF alteration is a BRAF mutation, a BRAF fusion, or a CRAF fusion. In some embodiments, the cancer has a non-V600 BRAF mutation. In some embodiments, the subject has a Class I BRAF mutation or a Class II BRAF mutation. In some embodiments, the subject lacks V600E mutation, V600K mutation, or both.

In some embodiments, the identifying step comprises identifying the subject with a fusion. In some embodiments, the subject is identified having one or more of the following fusions: KIAA1549:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, AGK:BRAF, AGAP3:BRAF, TNS3:BRAF, TARDBP:BRAF, ARMC10:BRAF, CUL1:BRAF, TRIM24:BRAF, AKAP9:BRAF, FKBP15:BRAF, SKAP2:BRAF, ZKSCAN1:BRAF, KLHL7:BRAF, SEPT3:BRAF, SRGAP3:RAF1, QK1:RAF1, FYCO:RAF1, ATG7:RAF1, and NFIA:RAF1. In some embodiments, the subject is identified as having one or more of the following fusions: AGK:BRAF, AGAP3:BRAF, TNS3:BRAF, or KIAA1549:BRAF. In some embodiments, the subject is identified as having a AGAP3:BRAF fusion. In some embodiments, the subject is identified as having a SRGAP3:RAF1 fusion. In some embodiments, the subject is identified having KIAA1549:BRAF fusion. In some embodiments, the MEK inhibitor is N-((S)-2,3-Dihydroxy-propyl)-3-(2-fluoro-4-iodo-phenylamino)-isonicotinamide (pimasertib) or a pharmaceutically acceptable salt thereof. In some embodiments, the MEK inhibitor is a compound as disclosed herein.

In some embodiments, the identifying step comprises, identifying the subject with a non V600 BRAF mutation. In some embodiments, the non V600 BRAF mutation is selected from: V600E, G469A, G464V, G466V, K601E, G469R, and L597R. In some embodiments, the non V600 BRAF mutation is selected from: V600E, G464A, G464V, K601E, and G469R. In some embodiments, the non V600 BRAF mutation is selected from: G464V, K601E, G469A, and G466V.

In some embodiments, the identifying step comprises, identifying the subject with a RAS mutation. In some embodiments, the RAS mutation is a KRAS mutation. In some embodiments, the KRAS mutation is selected from: KRAS G12C, KRAS G12V, KRAS G12D, KRAS Q61K, KRAS Q61H, KRAS G13D, and KRAS G12S. In some embodiments, the KRAS mutation is selected from: KRAS G12C, KRAS G12D, KRAS G13D, and KRAS G12S.

In some embodiments, the identifying step comprises, identifying the subject with a low-grade glioma (LGG). In some embodiments, the identifying step comprises, identifying the subject with a newly diagnosed LGG.

In some embodiments, the subject has not had a current or previous central serous retinopathy, retinal vein occlusion, or ophthalmopathy, unstable neurological condition, uncontrolled cardiovascular condition, or administered any pan-RAF inhibitor. In some embodiments, the subject has not been previously administered a pan-RAF therapy. In some embodiments, the subject is not concurrently receiving other chemotherapeutic agents (traditional chemotherapy, targeted agents, monoclonal antibodies, etc.), drugs with immunosuppressant properties (other than steroids).

In some embodiments, the subject has not received any prior therapies for treating cancer. In some embodiments, a method described herein is used as a front-line therapy for treating cancer.

In some embodiments, the subject in need thereof is from about 6 months to 25 years old. In some embodiments, the subject in need thereof is from about 1 year to 25 years old. In some embodiments, a subject in need thereof is 25 years of age of less. In some embodiments, a subject in need thereof is 20 years of age or less. In some embodiments, a subject in need thereof is 15 years of age or less. In some embodiments, a subject in need thereof is 10 years of age or less. In some embodiments, a subject in need thereof is 20, 19, 18, 17, 16, 15, 14, 13, 12, 11, 10 years of age or less. In some embodiments, the subject in need thereof is 1, 2, 3, 4, 5, 6, 7, 8 , 9, 10, 11, 12, 13, 14, 15, 16, 17, 18, 19, 20, 21, 22, 23, 24, 25 years old. In some embodiments, the subject in need thereof is less than 18 years old. In some embodiments, the subject in need thereof is at least 18 years old. In some embodiments, the subject in need thereof is older than 18 years old.

In some embodiments, a dose of Compound A or a pharmaceutically acceptable salt thereof required to achieve IC80 of pERK inhibition as measured by PMA-induced peripheral blood mononuclear cell (PBMC) is at least about 5%, about 10%, about 15%, about 20%, about 30%, about 40%, about 50%, about 60%, about 70%, about 80%, or about 90% lower than the dose of Compound A or a pharmaceutically acceptable salt thereof that is required in a monotherapy to achieve the same IC80 of pERK inhibition. In some embodiments, a dose of Compound A or a pharmaceutically acceptable salt thereof required to achieve IC80 of pERK inhibition as measured by PMA-induced peripheral blood mononuclear cell (PBMC) is at least about 20% lower than the dose of Compound A or a pharmaceutically acceptable salt thereof that is required in a monotherapy to achieve the same IC80 of pERK inhibition. In some embodiment, the dose of Compound A or a pharmaceutically acceptable salt thereof is a daily dose. In some embodiment, the dose of Compound A or a pharmaceutically acceptable salt thereof is weekly dose. In some embodiments, a weekly dose of Compound A or a pharmaceutically acceptable salt thereof required to achieve IC80 of pERK inhibition as measured by PMA-induced peripheral blood mononuclear cell (PBMC) is at least about 5%, about 10%, about 15%, about 20%, about 30%, about 40%, or about 50% lower than the weekly dose of Compound A or a pharmaceutically acceptable salt thereof that is required in a monotherapy to achieve the same IC80 of pERK inhibition. In some embodiments, a dose of the MEK inhibitor or a pharmaceutically acceptable salt thereof required to achieve IC80 of pERK inhibition as measured by PMA-induced peripheral blood mononuclear cell (PBMC) is at least about 5%, about 10%, about 15%, about 20%, about 30%, about 40%, about 50%, about 60%, about 70%, about 80%, or about 90% lower than the dose of the MEK inhibitor or a pharmaceutically acceptable salt thereof that is required in a monotherapy to achieve the same IC80 of pERK inhibition. In some embodiments, a dose of the MEK inhibitor or a pharmaceutically acceptable salt thereof required to achieve IC80 of pERK inhibition as measured by PMA-induced peripheral blood mononuclear cell (PBMC) is at least about 20% lower than the dose of the MEK inhibitor or a pharmaceutically acceptable salt thereof that is required in a monotherapy to achieve the same IC80 of pERK inhibition. In some embodiments, a dose of the MEK inhibitor or a pharmaceutically acceptable salt thereof required to achieve IC80 of pERK inhibition as measured by PMA-induced peripheral blood mononuclear cell (PBMC) is at least about 5%, about 10%, about 15%, about 20%, about 30%, about 40%, or about 50% lower than the dose of the MEK inhibitor or a pharmaceutically acceptable salt thereof that is required in a monotherapy to achieve the same IC80 of pERK inhibition. In some embodiment, the dose of the MEK inhibitor or a pharmaceutically acceptable salt thereof is daily dose. In some embodiment, the dose of the MEK inhibitor or a pharmaceutically acceptable salt thereof is weekly dose. IC80 of pERK inhibitor can be measured by a suitable method known in the art, e.g., as described in Adelmann et al, Oncotarget. 2016 May 24; 7(21): 30453-30460.

### EXAMPLES

The invention now being generally described, it will be more readily understood by reference to the following examples which are included merely for purposes of illustration of certain aspects and embodiments of the present invention, and are not intended to limit the invention in any way.

### Example 1: Identification of Markers

Markers with alterations or mutations can be identified through molecular assays as routinely performed at Clinical Laboratory Improvement Amendments of 1988 (CLIA) or through other similarly certified laboratories locally. Specifically, markers can be identified for any one of the following alterations or mutations: RAF alteration, a RAS mutation, an NF-1 mutation or a genomic alteration that results in a dependence on signaling through the MAPK pathway.

### Example 2: Treatment Using RAF Inhibitors and MEK Inhibitors

Compound A in combination with a MEK inhibitor as described herein, may be evaluated using a mutant cell model or mutant cancer cell model. An animal model may be inoculated with tumor cells for tumor development. Weight and tumor growth can be monitored during the tumor development. The inoculated subject may be treated with Compound A, a MEK inhibitor as described herein, or a combination of Compound A and the MEK inhibitor as described herein. A tumor suppression score can be determined using statistical tests to examine the differences between a control group and treatment group. The Bliss Independence Analysis may be used for both the 2D and 3D combination assays, in which a score above 0 indicates synergy whereas a score below 0 indicated antagonism. A score of 0 indicates additive.

### Example 3: Synergistic Effect Using RAF Inhibitors and MEK Inhibitors in Non V600 BRAF Mutant Tumor Cell Lines

For the human tumor cell lines, synergy was assessed in a 5×5 matrix combination format in a CellTiter-Glo based 2D monolayer assay. All compounds were added once at the start of the experiment, 24 hr after cell seeding. Cultures were incubated at 37° C. and 5% CO2 in a humidified incubator. The duration of compound treatment was 72 hr. Viability of the cells were analyzed by using CellTiter-Glo reagent in which luminescence was measured by using the EnVision Xcite multilabel plater reader. Synergy was measured by using Bliss independence Analysis.

In the 3D clonogenic assay, tumor cell suspensions were prepared directly from human non V600 BRAF mutant tumor xenografts growing in nude mice. These ex vivo PDX models were assessed in a 5×5 matrix combination format in a 3D clonogenic assay using ultra low attachment plates in which cells were mixed with cell culture media and soft agar. Compounds were added 24 hr after cell seeding. Cultures were incubated at 37 C and 7.5% CO % in a humidified incubator for 8-13 days and monitored closely for colony growth using an inverted microscope. Compound A was added every 2-3 days. After 8-13 days, vital colonies were stained for 48 hr with a sterile aqueous solution of 2-(4-iodophenyl)-3-(4-nitrophenyl)-5-phenyltetrazolium chloride (INT, 1 mg/ml, 25 μl/well), and colony counts were performed with an automatic image analysis system (Bioreader 5000 Vα, BIO-SYS). Synergy was measured using Bliss Independent Analysis.

The Bliss Independence Analysis was used for both the 2D and 3D combination assays, in which a score above 0 indicated synergy whereas a score below 0 indicated antagonism.

Table 1 demonstrates the observed response between Compound A and MEK inhibitors in non V600 BRAF mutant tumor cell lines.

TABLE 1
Synergy observed in response to Compound A and MEK inhibitors in non V600 BRAF
mutant tumor cell lines in vitro or PDX models ex vivo

| | | | BRAF | | Compound | Compound |
|---|---|---|---|---|---|---|
| | | BRAF | mutation | Tumor | A + | A + |
| Models | Name | mutation | class | type | binimetinib | selumetinib |
| Cell | A375 | V600E | 1 | Melanoma | 5 | 2 |
| lines | NCI- | G469A | 2 | Lung | 11 | 12 |
| (2D) | H1755 | | | | | |
| | MDA- | G464V | 2 | Breast | 20 | 14 |
| | MB- | | | | | |
| | 231 | | | | | |
| | NCI- | G466V | 3 | Lung | 1 | 2 |
| | H1666 | | | | | |
| PDX | MEXF | V600E | 1 | Melanoma | 3 | 3 |
| models | 2104 | | | | | |
| (3D) | MEXF | K601E | 2 | Melanoma | 12 | 8 |
| | 1870 | | | | | |
| | MEXF | G469R | 2 | Melanoma | 5 | 4 |
| | 622 | | | | | |

### Example 4: Synergistic Effect Using RAF Inhibitors and MEK Inhibitors in KRAS Mutant Tumor Cell Lines

Synergy was assessed in a 6×6 matrix combination format using a CellTiter-Glo based 2D monolayer assay. All compounds were added 24 hr after cell seeding. Compound A was added on days 2 and 3. The duration of compound treatment was 72 hr. Viability was measured using CellTitre-Glo in which luminescence was measured using an EnVision Multi Label Reader. Synergy was measured by Combination Index. Synergy scores were calculated both Bliss independence and Loewe additivity model. A score higher than 5 indicated synergy and a score less than −5 indicated antagonism.

Table 2 demonstrates the observed response between Compound A and MEK inhibitors in KRAS mutant tumor cell lines.

TABLE 2
Synergy observed in response to Compound A and MEK inhibitors
in KRAS mutant tumor cell lines in vitro.

| | | | Compound A + |
|---|---|---|---|
| Mutation | Cell lines | Tumor type | selumetinib |
| KRAS G12C | NCI-H358 | Lung | 5.585 |
| | NCI-H1792 | Lung | 40.876 |
| | Calu-1 | Lung | 7.57 |
| | NCI-H23 | Lung | 15.717 |
| | NCI-H2122 | Lung | 15.04 |
| | SW756 | Cervix | 17.091 |
| KRAS G12V | SW620 | Colon | 5.401 |
| | SW480 | Colon | 13.554 |
| | Capan-2 | Pancreas | 9.83 |
| | NCI-H441 | Lung | 5.012 |
| | NCI-H727 | Lung | 6.87 |
| KRAS G12D | LS513 | CRC | 22.344 |
| | HPAC-1 | Pancreas | 8.84 |
| KRAS Q61 | NCI-H460 | Lung | 11.098 |
| | Calu6 | Lung | 30.204 |
| KRAS G13D/ | HCT116 | Colon | 18.9 |
| PIK3CA-H1047R | | | |
| KRAS G12S | A549 | Lung | 9.212 |
| BRAF V600E | A375 | Melanoma | 6.21 |

### Example 5: Synergistic Effect Using Compound A and Pimasertib in 3D PDX Models Ex Vivo

In a 3D PDX ex vivo assay, tumor cell suspensions were prepared directly from human BRAF fusion tumor xenografts growing in nude mice. These ex vivo PDX models were assessed in a 5×5 matrix combination format in a 3D growth assay using ultra low attachment plates in which cells were mixed with cell culture media and 1% methylcellulose. A Day 0 luminescence reading following the addition of Cell-Titer Glo was taken 24 hours after seeding. Control and test Compounds were added 24 hr after cell seeding. Cultures were incubated at 37 C and 7.5% CO % in a humidified incubator for 7 days and monitored closely for growth and viability using an inverted microscope. Compound A and Pimasertib were added once at Day 0. After 7 days, Cell Titer Glo reagent (Promega) was added to each well, allowed to incubate and the luminescence was read. Synergy is calculated based on Crownsyn, which is an effect-based method developed by CrownBio under the assumption that both drugs act independently.

Table 3 demonstrates the observed synergy response between Compound A and pimasertib in BRAF fusion PDX models ex vivo.

TABLE 3
Synergy observed in response to Compound A and Pimasertib
in BRAF fusion PDX models

| CR6253 | | MU21652 | | PA3546 | | ME11971 | |
|---|---|---|---|---|---|---|---|
| AGAP3- | | KIAA1549- | | TNS3- | | AGK- | |
| BRAF, | | BRAF, | | BRAF, | | BRAF, | |
| CRC | | mixed mullerian | | panc | | melanoma | |
| Bliss | Loewe | Bliss | Loewe | Bliss | Loewe | Bliss | Loewe |
| 18.9 | 26.6 | 39.9 | 40.5 | 23.5 | 25.7 | 9.2 | 9.4 |

### Example 6: Synergistic Effect Using Compound A and Pimasertib in 3D Organoid Model

In a 3D organoid assay, the requisite number of organoids were processed 1:1 using 50% Matrigel to define the right size of organoids used in the screen. On Day 0 organoids were recovered from all the wells, filtered and seeded. Matrigel was added to a final concentration of 5%. Compounds were added at seeding after the organoids had settled. Cultures were incubated at 37 C and 7.5% CO % in a humidified incubator for 5 days and monitored closely for growth and viability using an inverted microscope. Compound A and Pimasertib were added once at Day 0. After 5 days, Cell Titer Glo reagent (Promega) was added to each well, allowed to incubate and the luminescence was read. Synergy is calculated based on Crownsyn, which is an effect-based method under the assumption that both drugs act independently.

Table 4 demonstrates the observed response between Compound A and Pimasertib in PDX Organoid Model. The synergy score of Table 4 is calculated using Loewe algorithm where a score higher than 5 indicates synergy and a score less than 5 indicates antagonism.

TABLE 4
Synergy observed in response to Compound A and Pimasertib in
colon PDX organoid model harboring an AGAP3-BRAF fusion

| Combination | AGAP3-BRAF; BRAF-AGAP3 |
|---|---|
| Compound A and Pimasertib | 10.8 |

### Example 7: Synergistic Effect Using Compound A and Pimasertib in Non V600 BRAF Mutant Cell Lines in Vitro (2D) or PDX Models Ex Vivo (3D)

In the 2D cell proliferation monolayer assay, cells were seeded for and treated for 72 hours. All compounds were added once at the start of the experiment. Compound A was repeatedly applied every 24 hours. Cultures were incubated at 37 C and 7.5% CO % in a humidified incubator for 72 hours and monitored closely for growth and viability using an inverted microscope. After 72 hours Cell Titer Glo reagent (Promega) was added to each well, allowed to incubate and the luminescence was read.

In the 3D clonogenic assay, repeated application of Compound A was done every 2-3 days whereas pimasertib was only added once at the start of the experiment. At maximum colony formation, between 8-13 days, colony counts were performed.

Table 5 demonstrates the observed synergy response between Compound A and Pimasertib in non V600 BRAF mutant tumor cell lines in vitro (2D) or PDX models ex vivo (3D). A positive number indicated synergy as the number of combination pairs which achieved a Bliss Index≥0.15. A O score indicated additive effects with Bliss Index between −0.15 and +0.15 for all combination pairs. A negative number indicated antagonism as the number of combination pairs which achieved a Bliss Index of ≤−0.15.

Table 6 demonstrates the observed synergy response between Compound A and Pimasertib in non V600 BRAF mutant tumor cell lines in vitro (2D). Synergy was assessed in a 6×6 matrix combination format Combination Index synergy scoring was determined, and Loewe score is presented. A score higher than 5 indicated synergy and a score less than 5 indicated antagonism.

Table 7 demonstrates the observed synergy response between Compound A and Pimasertib in KRAS mutant cell lines in vitro (2D). Synergy was assessed in a 5×5 matrix combination format followed by Bliss independence analysis. A positive number indicated synergy as the number of combination pairs which achieved a Bliss Index≥0.15. A O score indicated additive effects with Bliss Index between −0.15 and +0.15 for all combination pairs. A negative number indicated antagonism as the number of combination pairs which achieved a Bliss Index of ≤−0.15.

TABLE 5
Synergy observed in response to Compound A and Pimasertib in
non V600 BRAF mutant tumor cell lines in vitro (2D) or PDX
models ex vivo (3D)

| | | | BRAF | | |
|---|---|---|---|---|---|
| Assay | | BRAF | mutation | Tumor | Synergy |
| Format | Cell Line | mutation | class | type | Score |
| Cell lines | NCI-H1755 | G469A | 2 | Lung | 20 |
| (2D) | MDA-MB-231 | G464V | 2 | Breast | 3 |
| | NCI-H1666 | G466V | 3 | Lung | −2 |
| | 22RV1 | L597R | 2 | Prostate | −2 |
| | Cal12T | G466V | 2 | Lung | −2 |
| | OV90 | Indel | 2 | Ovarian | 0 |
| | BxPC3 | Indel | 2 | Pancreatic | 0 |
| PDX | MEXF 2104 | V600E | 1 | Melanoma | N/A |
| models | MEXF 1870 | K601E | 2 | Melanoma | 13 |
| (3D) | MEXF 622 | G469R | 2 | Melanoma | 8 |
| | MEXF 1876 | G496V | 2 | Pancreatic | −2 |

TABLE 6
Synergy observed in response to Compound A and Pimasertib in
non V600 BRAF mutant tumor cell lines in vitro (2D)

| | | | BRAF | | |
|---|---|---|---|---|---|
| Assay | | BRAF | mutation | Tumor | Synergy |
| Format | Cell Line | mutation | class | type | Score |
| Cell lines | NCI-H1755 | G469A | 2 | Lung | −0.6 |
| (2D) | MDA-MB-231 | G464V | 2 | Breast | 11.8 |
| | BxPC3 | Indel | 2 | Pancreatic | 25.7 |
| | NCI-H2087 | L597R | 2 | Lung | 5.5 |
| | NCI-H1666 | G466V | 3 | Lung | 12.5 |

TABLE 7
Synergy observed in response to Compound A and Pimasertib in
KRAS mutant cell lines in vitro (2D) .

| | | Tumor | |
|---|---|---|---|
| Cell Line | Mutation | type | Synergy Score |
| NCI-H1755 | KRAS G12C | Lung | 15 |
| NCI-H2122 | KRAS G12C | Lung | 12 |
| NCI-H23 | KRAS G12C | Lung | 0 |
| SW1573 | KRAS G12C | Lung | 0 |
| Calu1 | KRAS G12C | Lung | −7 |
| NCI-H358 | KRAS G12C | Lung | −1 |
| SW756 | KRAS G12C | Cervix | −4 |
| MiaPaCa2 | KRAS G12C | Pancreas | −1,1 |
| SW480 | KRAS G12V | Colon | 0 |
| SW620 | KRAS G12V | Colon | 0 |
| NCI-H441 | KRAS G12V | Lung | −3 |
| NCI-H727 | KRAS G12V | Lung | 0 |
| Capan-2 | KRAS G12V | Pancreas | −5 |
| LS513 | KRAS G12D | Colon | 1 |
| HPAC | KRAS G12D | Pancreas | −3 |
| A549 | KRAS G12S | Lung | 3 |
| HCT116 | KRAS G12D | Colon | 13 |
| | PIK3CA H1047R | | |
| Calu6 | KRAS Q61K | Lung | −1,4 |
| NCI-H460 | KRAS Q61H | Lung | 0 |
| A375 | BRAF V600E | Melanoma | −5 |

### Example 8: Treatment Schedule, Inclusion and Exclusion Criteria for the Combination of Compound A and Pimasertib

### Part 1

This is a multi-center, open-label sub-study of patients≥12 years of age, with recurrent or progressive solid tumors with aberrations in the key proteins of the MAPK pathway, such as tumors that harbor RAS or RAF alterations. Patients with these alterations will be identified through molecular assays routinely performed at Clinical Laboratory Improvement Amendments of 1988 (CLIA) or other similarly certified laboratories locally.

The study will consist of a screening period, a treatment period, a safety follow-up period, and a long-term follow-up period where survival, and subsequent anticancer therapies will be collected.

Compound A will be administered once weekly (Days 1, 8, 15, and 22) and pimasertib will initially be administered twice daily (BID). Patients will undergo radiographic evaluation of their disease at the end of every 2 cycles for 1 year and then every 3 cycles thereafter. Patients will continue on Compound A plus pimasertib until radiographic evidence of disease progression by criteria as appropriate for their disease setting, unacceptable toxicity, patient withdrawal of consent, or death. Generally, response assessment will be performed according to RECIST version 1.1 for solid tumors. Alternative criteria may be used in specific disease settings, such as glioma, where response assessment will be assessed by RANO criteria. Patients who have radiographic evidence of disease progression may be allowed to continue Compound A plus pimasertib if, in the opinion of a medical professional, the patient is deriving clinical benefit from continuing study treatment of the combination.

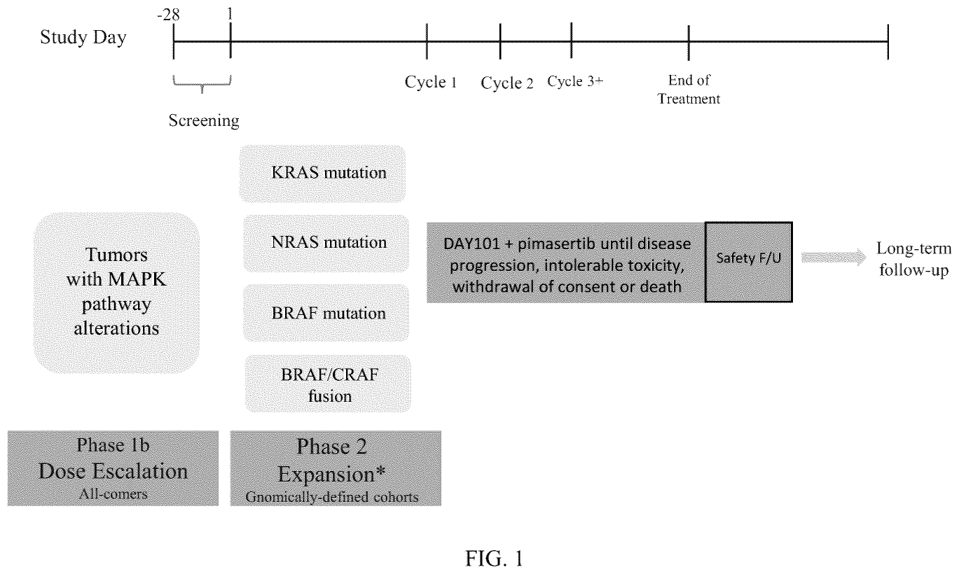

FIG. 1. provides the study design for the treatment of patients≥12 years of age, with recurrent or progressive solud tumors with aberrations in the key protein of the MAPK pathway, such as tumors that harbor RAS and RAF alterations. The study will consist of a screening period, a treatment period, a safety follow-up period, and a long-term follow-up period where survival, and subsequent anticancer therapies will be collected. Compound A will be administered once weekly (Days 1, 8, 15, and 22) and pimasertib will be administered once (QD) or twice daily (BID). The doses of Compound A and pimasertib will be determined by the dose cohort the patient is assigned to in the Phase 1b portion of the study. The doses and schedules of Compound A and pimasertib to be administered in the Phase 2 portion of the study will be determined during the Phase 1b portion. Cycles repeat every 28 days in the absence of disease progression or unacceptable toxicity. Patients will undergo radiographic evaluation of their disease at the end of every 2 cycles for 1 year and then every 3 cycles thereafter. Patients will continue on Compound A plus pimasertib until radiographic evidence of disease progression by criteria as appropriate for their disease setting, unacceptable toxicity, patient withdrawal of consent, or death.

A Bayesian optimal interval (BOIN) design with the 3+3 design run-in will be utilized for dose escalation of Compound A and pimasertib in the Dose escalation portion of the study.

The inclusion criteria can include one or more of the following: a confirmed MAPK pathway aberration, an ECOG performance status 0-1, and adequate organ function.

Exclusion criteria can include one or more of the following: current or previous central serous retinopathy, retinal vein occlusion, or ophthalmopathy; unstable neurological condition, despite adequate treatment; uncontrolled cardiovascular condition; and prior receipt of any pan-RAF inhibitor.

Exclusion criteria can also include: (a) Prior receipt of any pan-RAF inhibitor therapy (e.g., LXH254/naporafenib, BGB-283, BGB-3245, belvarafenib), and/or (b) concomitant medications which are strong inhibitors of cytochrome P450 CYP3A4 or CYP2C19, strong inducers of CYP3A4, or substrates of CYP2C9 with a narrow therapeutic index.

### Part 2

The recommended doses of Compound A and pimasertib in combination will be determined after review of the part 1 safety, efficacy and pharmacodynamic assessments.

In this part 2 dose-expansion portion, patients will be enrolled into biomarker-defined expansion cohorts to receive the combination, with the number of cohorts determined after analysis of the part 1 data. Patients will be enrolled into biomarker-defined (For example, NRAS, KRAS, HRAS, BRAF mutation, BRAF/CRAF fusion-positive, CRAF-amplified solid tumors, and the like) expansion cohorts to receive the combination of Compound A and Pimasertib. The primary endpoint will be the overall response rate, estimated for each cohort, as assessed by medical professionals. Secondary endpoints include safety and tolerability, duration of response, progression-free survival, overall survival and pharmacokinetics.

Table 8 provides treatment regimens for the Combination of Compound A and Pimasertib.

TABLE 8
Dosing Levels For The Combination of Compound A and Pimasertib

| Dose level | Compound A | Pimasertib |
|---|---|---|
| −2 | Adults: 200 mg QW | 15 mg QD |
| | Adolescents: 140 mg/m² | |
| −1 | Adults: 200 mg QW | 15 mg BID |
| | Adolescents: 140 mg/m² | |
| 1 | Adults: 400 mg QW | 15 mg BID |
| (starting dose level) | Adolescents: 280 mg/m² | |
| 2 | Adults: 600 mg QW | 15 mg BID |
| | Adolescents: 420 mg/m² | |
| 3 | Adults: 600 mg QW | 30 mg BID |
| | Adolescents: 420 mg/m² | |
| 4 | Adults: 600 mg QW | 45 mg BID |
| | Adolescents: 420 mg/m² | |
| 5 | Adults: 600 mg QW | 60 mg BID |
| | Adolescents: 420 mg/m² | |

BID = twice daily;
QD = once daily;
QW = once weekly.
Adults: ≥ 18 years of age.
Adolescents: ≥ 12 to < 18 years of age, BSA ≥ 1.3 m².

### Example 9: Dose Modification for Compound A and Pimasertib

The doses for Compound A in a method described herein can modified, e.g., as illustrated in the tables below. Dose levels may be modified for individual patients to manage toxicity.

Table 8 provides dose modification of Compound A for adults (≥18 Years of Age). Up to two dose reductions may be permitted from the starting dose.

Table 9 provides dose modifications of Compound A for adolescents (≥12-17 Years of Age). BSA should be calculated, and an updated dose should be provided on Day 1 of each cycle. BSA can be determined by any suitable calculation method. In some embodiments, the BSA is determined by Mosteller Formula (√((height×weight)/3600)). In some embodiments, the BSA is determined at the start of each cycle of administration.

Table 10 provides dose modifications of Pimasertib. Up to two dose reductions may be permitted from the starting dose.

TABLE 8
Compound A Dose Levels for Dose Modification in
Adults ≥ 18 Years of Age

| Compound A Starting Dose | Dose Modification |
|---|---|
| 600 mg QW | 1^{st} dose reduction: 500 mg QW |
| | 2^{nd} dose reduction: 400 mg QW |
| 400 mg QW | 1^{st} dose reduction: 300 mg QW |
| | 2^{nd} dose reduction: 200 mg QW |
| 200 mg QW | 1^{st} dose reduction: 100 mg QW |
| | 2^{nd} dose reduction: 60 mg QW |

QW = every week.

TABLE 9
Compound A Dose Rounding Guidelines in Adolescents (>12-17 Years of Age)

| | 420 | 350 | 280 | 210 | 140 | 70 | 35 |
|---|---|---|---|---|---|---|---|
| BSA | mg/m² | mg/m² | mg/m² | mg/m² | mg/m² | mg/m² | mg/m² |
| (m²) | (mg) | (mg) | (mg) | (mg) | (mg) | (mg) | (mg) |
| 0.9-1.0 | 400 | 300 | 300 mg | 200 | 100 | 60 | 20 mg |
| | | | alternating | | | | alternating |
| | | | with 200 | | | | with 40 |
| | | | mg QOW | | | | mg QOW |
| 1.1-1.2 | 500 | 400 | 300 | 300 mg | 200 mg | 100 mg | 40 |
| | | | | alternating | alternating | alternating | |
| | | | | with 200 | with 100 | with 60 | |
| | | | | mg QOW | mg QOW | mg QOW | |
| ≥1.3 | 600 | 500 | 400 | 300 | 200 | 100 | 60 |

BSA = body surface area. BSA can be determined by any suitable calculation method. In some embodiments, the BSA is determined by Mosteller Formula (√((height × weight)/3600)). In some embodiments, the BSA is determined at the start of each cycle of administration.
QOW = every other week.

TABLE 10
Pimasertib Dose Levels for Dose Level Modification

| Pimasertib Starting Dose | Dose Modification |
|---|---|
| 60 mg BID | 1^{st} dose reduction: 45 mg BID |
| | 2^{nd} dose reduction: 30 mg BID |
| 45 mg BID | 1^{st} dose reduction: 30 mg BID |
| | 2^{nd} dose reduction: 15 mg BID |
| 30 mg BID | 1^{st} dose reduction: 15 mg BID |
| | 2^{nd} dose reduction: 15 mg BID 5 days |
| | on, 2 days off |
| 15 mg BID | 1^{st} dose reduction: 15 mg BID 5 days |
| | on, 2 days off |
| | 2^{nd} dose reduction: 15 mg QD |
| 15 mg BID 5 days on, 2 days off | 1^{st} dose reduction: 15 mg QD |
| | 2^{nd} dose reduction: 15 mg QD 5 days |
| | on, 2 days off |

In some cases, when administered in a form of pimasertib salt such as pimasertib HCl, the dose described in Table 10 represents the weight of the pimasertib free base present in the salt.

### Example 10. Evaluation of the Preclinical Efficacy of Compound A and Pimasertib as Single Agents in a BRAF Fusion Model of Melanoma (AGK-BRAF Fusion)

The preclinical evaluation will be conducted using the in vivo therapeutic efficacy of Compound A and Pimasertib as single agents in the treatment of melanoma cancer xenograft model ME11971 (AGK-BRAF fusion) in female NOD/SCID mice. Tumor fragments from stock mice will be harvested and used for inoculation into the mice. Each mouse will be inoculated subcutaneously in the right flank with ME11971 model tumor fragment (2-3 mm in diameter) for tumor development.

Table 11 provides the study mice study parameters: a total of 100 mice will be enrolled in the study and randomly allocated to 10 study groups with 10 mice per group. The randomization will start when the mean tumor size reaches approximately 150 (100-200) mm³. Randomization will be performed based on “Matched distribution” method/“Stratified” method (StudyDirector™ software, version 3.1.399.19) randomized block design. The date of randomization will be designated as day 0.

After tumor inoculation, the animals will be checked daily for morbidity and mortality. During routine monitoring, the animals will be checked for any effects of tumor growth and treatments on behavior such as mobility, food and water consumption, body weight gain/loss (Body weights will be measured twice per week after randomization), eye/hair matting and any other abnormalities. Mortality and observed clinical signs will be recorded for individual animals in detail. Tumor volumes will be measured twice per week after randomization. Dosing as well as tumor and body weight measurements will be conducted in a Laminar Flow Cabinet. The body weight of all animals will be monitored throughout the study. Animals will be euthanized if they lose over 15% of their body weight relative to the weight at the first day of treatment for 3 consecutive days or lose over 20% of their body weight relative to the weight at the first day of treatment.

The body weights and tumor volumes will be measured by using StudyDirector™ software (version 3.1.399.19). The treatment will be initiated one day post grouping (day 1) or on the same day of randomization (Day 0).

Mice will be dosed in groups of 10 as per Table 11 above for a period of 14 days. The three doses chosen for Compound A are 12.5 mg/kg, 25 mg/kg, and 50 mg/kg and for Pimasertib 10 mg/kg, 30 mg/kg, and 60 mg/kg. The study will be terminated when the mean tumor volume of the vehicle control group reaches 2000 mm³ or upon tumor and plasma samples collection after the final dose, whichever comes first. The treatment will be performed for 14 days. If there is no extension of the treatment, the study will be terminated 4 hours (Group 1, 2, 3, 4)/0.5 hour (Group 5, 6, 7, 8) after final dose.

TABLE 11
Preclinical Efficacy Study Design Of Compound A And Pimasertib As Single Agents
In Melanoma Cancer Xenograft Model ME11971 (AGK-BRAF fusion) In Female
NOD/SCID Mice

| | | | Dose | Dose | Dose | | | |
|---|---|---|---|---|---|---|---|---|
| | | | Level | Conc. | Vol. | | Dose | Endpoint |
| Group | N | Test Agent | (mg/kg) | (mg/ml) | (ml/kg) | ROA | Schedule | Collection |
| 1 | 10 | Vehicle 1 | NA | NA | 5 | PO | QD | Tumor, |
| | | | | | | | | plasma (4 hr) |
| 2 | 10 | Compound A | 12.5 | 2.5 | 5 | PO | QD | Tumor, |
| | | | | | | | | plasma (4 hr) |
| 3 | 10 | Compound A | 25 | 5.0 | 5 | PO | QD | Tumor, |
| | | | | | | | | plasma (4 hr) |
| 4 | 10 | Compound A | 50 | 10 | 5 | PO | QD | Tumor, |
| | | | | | | | | plasma (4 hr) |
| 5 | 10 | Vehicle 2 | NA | NA | 5 | PO | BID | Tumor, |
| | | | | | | | | plasma (4 hr) |
| 6 | 10 | Pimasertib | 10 | 2 | 5 | PO | BID | Tumor, |
| | | | | | | | | plasma (4 hr) |
| 7 | 10 | Pimasertib | 30 | 6 | 5 | PO | BID | Tumor, |
| | | | | | | | | plasma (4 hr) |
| 8 | 10 | Pimasertib | 60 | 12 | 5 | PO | BID | Tumor, |
| | | | | | | | | plasma (4 hr) |
| 9 | 10 | Vehicle 3 | NA | | 5 | IP | QW | NA |
| 10 | 10 | Cisplatin | NA | | 5 | IP | QW | NA |

### Example 11. Evaluation of the Preclinical Efficacy of Compound A and Pimasertib in Combination in a BRAF Fusion Model of Melanoma (AGK-BRAF Fusion)

The preclinical evaluation will be conducted using the in vivo therapeutic efficacy of Compound A and Pimasertib in combination in the treatment of melanoma cancer xenograft model ME11971 (AGK-BRAF fusion) in female NOD/SCID mice. Tumor fragments from stock mice will be harvested and used for inoculation into the mice. Each mouse will be inoculated subcutaneously in the right flank with ME11971 model tumor fragment (2-3 mm in diameter) for tumor development.

Table 11 provides the mice study parameters: a total of 70 mice will be enrolled in the study and randomly allocated to 10 study groups with 10 mice per group. The randomization will start when the mean tumor size reaches approximately 150 (100-200) mm³. Randomization will be performed based on “Matched distribution” method/“Stratified” method(StudyDirector™ software, version 3.1.399.19) randomized block design. The date of randomization will be designated as day 0.

After tumor inoculation, the animals will be checked daily for morbidity and mortality. During routine monitoring, the animals will be checked for any effects of tumor growth and treatments on behavior such as mobility, food and water consumption, body weight gain/loss (Body weights will be measured twice per week after randomization), eye/hair matting and any other abnormalities. Mortality and observed clinical signs will be recorded for individual animals in detail.

Tumor volumes will be measured twice per week after randomization. Dosing as well as tumor and body weight measurements will be conducted in a Laminar Flow Cabinet. The body weight of all animals will be monitored throughout the study. Animals will be euthanized if they lose over 15% of their body weight relative to the weight at the first day of treatment for 3 consecutive days or lose over 20% of their body weight relative to the weight at the first day of treatment.

The body weights and tumor volumes will be measured by using StudyDirector™ software (version 3.1.399.19). The treatment will be initiated one day post grouping (day 1) or on the same day of randomization (Day 0).

Mice will be dosed in groups of 10 as per the table above for a period of 14 days. The two doses chosen for Compound A are 12.5 mg/kg and 25 mg/kg, and for Pimasertib 10mg/kg. The study will be terminated when the mean tumor volume of the vehicle control group reaches 2000 mm³ or upon tumor and plasma samples collection after the final dose, whichever comes first. The treatment will be performed for 14 days. If there is no extension of the treatment, the study will be terminated at an endpoint that is gated on the single agent efficacy study.

TABLE 12
Preclinical Efficacy Study Design Using A Combination of Compound A And
Pimasertib In Melanoma Cancer Xenograft Model ME11971 (AGK-BRAF fusion)
In Female NOD/SCID Mice

| | Group | Group | Group | Group | Group | Group | Group |
|---|---|---|---|---|---|---|---|
| | 1 | 2 | 3 | 4 | 5 | 6 | 7 |
| N | 10 | 10 | 10 | 10 | 10 | 10 | 10 |
| Test | Vehicle | Compound | Compound | — | — | Compound | Compound |
| Article 1 | 1 | A | A | | | A | A |
| Test | — | — | — | Vehicle | Pimasertib | Pimasertib | Pimasertib |
| Article 2 | | | | 1 | | | |
| Test | NA | 12.5 | 25 | — | — | 12.5 | 25 |
| Article 1 | | | | | | | |
| Dose | | | | | | | |
| Level | | | | | | | |
| (mg/kg) | | | | | | | |
| Test | — | — | — | NA | 10 | 10 | 10 |
| Article 2 | | | | | | | |
| Dose | | | | | | | |
| Level | | | | | | | |
| (mg/kg) | | | | | | | |
| Test | 5 | 5 | 5 | — | — | 5 | 5 |
| Article 1 | | | | | | | |
| Dose Vol. | | | | | | | |
| (ml/kg) | | | | | | | |
| Test | — | — | — | 5 | 5 | 5 | 5 |
| Article 2 | | | | | | | |
| Dose Vol. | | | | | | | |
| (ml/kg) | | | | | | | |
| Test | — | — | — | — | — | 2.5 | 5 |
| Article 1 | | | | | | | |
| Dose | | | | | | | |
| Conc. | | | | | | | |
| (mg/ml) | | | | | | | |
| Test | — | — | — | — | — | 2 | 2 |
| Article 2 | | | | | | | |
| Dose | | | | | | | |
| Conc. | | | | | | | |
| (mg/ml) | | | | | | | |
| ROA | PO | PO | PO | PO | PO | PO | PO |
| Test | | | | | | | |
| Article | | | | | | | |
| 1/2 | | | | | | | |
| Dose | QD | QD | QD | BID | BID | QD/BID | QD/BID |
| Schedule | | | | | | | |
| Test | | | | | | | |
| Article | | | | | | | |
| 1/2 | | | | | | | |
| Endpoint | Tumor, | Tumor, | Tumor, | Tumor, | Tumor, | Tumor, | Tumor, |
| Collection | plasma | plasma | plasma | plasma | plasma | plasma | plasma |

Disclosure of the present application is further illustrated in the following list of embodiments, which are given for illustration purposes only and are not intended to limit the disclosure in any way:

### Additional Embodiments

Embodiment 1. A method of treating a subject suffering from cancer, comprising administering to the subject:
(i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof; and
(ii) a MEK inhibitor, wherein the MEK inhibitor is a compound having a structure of Formula (I) or a pharmaceutically acceptable salt thereof,

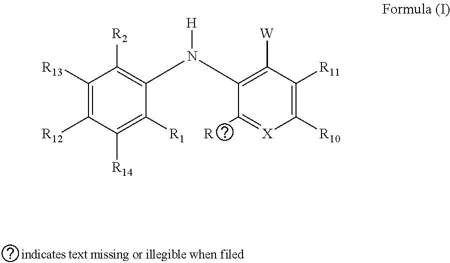

wherein,
R₁, R₂, R₉, R₁₀, R₁₁ R₁₂, R₁₃ and R₁₄ are independently selected from: hydrogen, halogen, cyano, nitro, azido, —OR₃, —NR₄C(O)OR₆, —OC(O)R₃, —NR₄S(O)ⱼR₆, —S(O)ⱼNR₃R₄, —S(O)ⱼNR₄C(O)R₃, —C(O)NR₄S(O)ⱼR₆, —S(O)ⱼR₆, —NR₄C(O)R₃, —C(O)NR₃R₄, —NR₅C(O)NR₃R₄, —NR₅C(NCN)NR₃R₄, —NR₃R₄, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR₄R₅)ₘ-aryl, aryl, arylalkyl, heteroaryl, heteroarylalkyl, heterocyclyl, heterocyclylalkyl, —O(CR₄R₅)ₘ-aryl, —NR₄(CR₄R₅)ₘ-aryl, —O(CR₄R₅)ₘ-heteroaryl, —NR₄(CR₄R₅)ₘ, heteroaryl, —O(CR₄R₅)ₘ-heterocyclyl, —NR₄(CR₄R₅)ₘ-heterocyclyl and —S(C₁-C₂ alkyl) substituted with 1 to 5 fluorines, wherein each alkyl, alkenyl, alkynyl, cycloalkyl, aryl, heteroaryl and heterocyclyl is independently substituted or unsubstituted;
R₃ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, arylalkyl, heteroaryl, heteroarylalkyl, heterocyclyl, heterocyclylalkyl, and aryl; wherein each alkyl, alkenyl, alkynyl, cycloalkyl, heteroaryl and heterocyclyl is independently substituted or unsubstituted; and wherein aryl is optionally substituted with 1 to 5 groups independently selected from: oxo, halogen, nitro, CF₃, CHF₂, CH₂F, OCF₃, OCHF₂, OCH₂F, azido, NR′SO₂R″″, SO₂N″, C(O)R′, C(O)OR′, OC(0)R′, NR′C(O)OR″″, NR′C(O)R″, C(O)NR′R″, SR″″, S(0)R″″, SO₂R′, NR′R″, NR′C(O)NR″R′, NR′C(NCN)N″R′″, OR′, aryl, heteroaryl, arylalkyl, heteroarylalkyl, heterocyclyl, and heterocyclylalkyl;
R₄ is selected from hydrogen or C₁₋₆ alkyl, wherein alkyl may be substituted or unsubstituted; or
R₃ and R₄ can be taken together with the atom to which they are attached to form a 4 to 10 membered heteroaryl or heterocyclic ring, each of which is substituted or unsubstituted;
R₅ is hydrogen or C₁-C₆ alkyl, wherein alkyl may be substituted or unsubstituted; or
R₄ and R₅ can be taken together with the atom to which they are attached to form a 4 to 10 membered carbocyclic, heteroaryl or heterocyclic ring, each of which is substituted or unsubstituted;
R₆ is selected from: trifluoromethyl, C₁-C₁₀ alkyl, C₃-C₁₀ cycloalkyl, aryl, arylalkyl, heteroaryl, heteroarylalkyl, heterocyclyl, and heterocyclylalkyl, wherein each alkyl, cycloalkyl, aryl, heteroaryl and heterocyclyl is independently substituted or unsubstituted;
R′, R″ and R″′ are independently selected from: hydrogen, C₁-C₄ alkyl, C₂-C₄ alkenyl, aryl and arylalkyl;
R″″ is selected from C₁-C₄ alkyl, C₂-C₄ alkenyl, aryl and arylalkyl;
W is selected from 1) heteroaryl containing 1-4 heteroatoms or herterocyclyl containing 1-4 heteroatoms each of which is unsubstituted or substituted by 1 to 5 substituents ZR₁₅; and 2) —C(O)OR₁₅, —C(O)NR₄R₁₅, —C(O)NR₄OR₁₅, —C(O)NR₄S(O)ⱼR₆, —C(O)NR₄NR₄NR₁₅, —NR′R″, —NR′C(O)R′, —NR′S(O)ⱼR′, —NRC(O)NR′R″, NR′S(O)ⱼNR′R″, or —C(O)NR₄NR₄C(O)R₁₅; provided that W is not —C(O)OH;
Z is a bond, NR₁₆, O, NR₁₆SO₂ or S;
R₁₅ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl; wherein each alkyl, alkenyl, alkynyl, cycloalkyl, aryl, heteroaryl and heterocyclyl is independently substituted or unsubstituted;
R₁₆ is selected from hydrogen or C₁-C₁₀ alkyl; or R₁₅ and R₁₆ taken together with the atom to which they are attached form a 4 to 10 membered cyclic ring with 1 or 2 nitrogen atoms and optionally an oxygen atom, said ring being substituted or unsubstituted;
X is N or N⁺O⁻
m is 0, 1, 2, 3,4 or 5; and
j is 1 or 2;
   wherein a total amount of the Compound A or a pharmaceutically acceptable salt thereof and the MEK inhibitor is therapeutically effective in treating the cancer.

Embodiment 2. A method of treating a subject suffering from cancer, comprising
(a) identifying a subject suffering from cancer, wherein the cancer has one or more of: a RAF alteration, a RAS mutation, an NF-1 mutation, or a genomic alteration that results in a dependence on signaling through the MAPK pathway; and
(b) administering to the subject
   (i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof; and
   (ii) a MEK inhibitor, wherein the MEK inhibitor is a compound having a structure of Formula (I) or a pharmaceutically acceptable salt thereof,

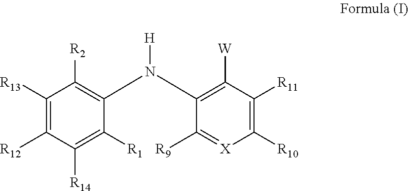

wherein,
R₁, R₂, R₉, R₁₀, R₁₁ R₁₂, R₁₃ and R₁₄ are independently selected from: hydrogen, halogen, cyano, nitro, azido, —OR₃, —NR₄C(O)OR₆, —OC(O)R₃, —NR₄S(O)ⱼR₆, —S(O)ⱼNR₃R₄, —S(O)ⱼNR₄C(O)R₃, —C(O)NR₄S(O)ⱼR₆, —S(O)ⱼR₆, —NR₄C(O)R₃, —C(O)NR₃R₄, —NR₅C(O)NR₃R₄, —NR₅C(NCN)NR₃R₄, —NR₃R₄, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR₄R₅)ₘ-aryl, aryl, arylalkyl, heteroaryl, heteroarylalkyl, heterocyclyl, heterocyclylalkyl, —O(CR₄R₅)ₘ-aryl, —NR₄(CR₄R₅)ₘ-aryl, —O(CR₄R₅)ₘ-heteroaryl, —NR₄(CR₄R₅)ₘ, heteroaryl, —O(CR₄R₅)ₘ-heterocyclyl, —NR₄(CR₄R₅)ₘ-heterocyclyl and —S(C₁-C₂ alkyl) substituted with 1 to 5 fluorines, wherein each alkyl, alkenyl, alkynyl, cycloalkyl, aryl, heteroaryl and heterocyclyl is independently substituted or unsubstituted;
R₃ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, arylalkyl, heteroaryl, heteroarylalkyl, heterocyclyl, heterocyclylalkyl, and aryl; wherein each alkyl, alkenyl, alkynyl, cycloalkyl, heteroaryl and heterocyclyl is independently substituted or unsubstituted; and wherein aryl is optionally substituted with 1 to 5 groups independently selected from: oxo, halogen, nitro, CF₃, CHF₂, CH₂F, OCF₃, OCHF₂, OCH₂F, azido, NR′SO₂R″″, SO₂N″, C(O)R′, C(O)OR′, OC(0)R′, NR′C(O)OR″″, NR′C(O)R″, C(O)NR′R″, SR″″, S(0)R″″, SO₂R′, NR′R″, NR′C(O)NR″R″′, NR′C(NCN)N″R″′, OR′, aryl, heteroaryl, arylalkyl, heteroarylalkyl, heterocyclyl, and heterocyclylalkyl;
R₄ is selected from hydrogen or C₁₋₆ alkyl, wherein alkyl may be substituted or unsubstituted; or
R₃ and R₄ can be taken together with the atom to which they are attached to form a 4 to 10 membered heteroaryl or heterocyclic ring, each of which is substituted or unsubstituted;
R₅ is hydrogen or C₁-C₆ alkyl, wherein alkyl may be substituted or unsubstituted; or R₄ and R⁵ can be taken together with the atom to which they are attached to form a 4 to 10 membered carbocyclic, heteroaryl or heterocyclic ring, each of which is substituted or unsubstituted;
R₆ is selected from: trifluoromethyl, C₁-C₁₀ alkyl, C₃-C₁₀ cycloalkyl, aryl, arylalkyl, heteroaryl, heteroarylalkyl, heterocyclyl, and heterocyclylalkyl, wherein each alkyl, cycloalkyl, aryl, heteroaryl and heterocyclyl is independently substituted or unsubstituted;
R′, R″ and R″′ are independently selected from: hydrogen, C₁-C₄ alkyl, C₂-C₄ alkenyl, aryl and arylalkyl;
R″″ is selected from C₁-C₄ alkyl, C₂-C₄ alkenyl, aryl and arylalkyl;
W is selected from 1) heteroaryl containing 1-4 heteroatoms or herterocyclyl containing 1-4 heteroatoms each of which is unsubstituted or substituted by 1 to 5 substituents ZR₁₅; and 2) —C(O)OR₁₅, —C(O)NR₄R₁₅, —C(O)NR₄OR₁₅, —C(O)NR₄S(O)ⱼR₆, —C(O)NR₄NR₄NR₁₅, —NR′R″, —NR′C(O)R′, —NR′S(O)ⱼR′, —NRC(O)NR′R″, NR′S(O)ⱼNR′R″, or —C(O)NR₄NR₄C(O)R₁₅; provided that W is not —C(O)OH;
Z is a bond, NR₁₆, O, NR₁₆SO₂ or S;
R₁₅ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, aryl, arylalkyl, heteroaryl, heteroarylalkyl, heterocyclyl, and heterocyclylalkyl; wherein each alkyl, alkenyl, alkynyl, cycloalkyl, aryl, heteroaryl and heterocyclyl is independently substituted or unsubstituted;
R₁₆ is selected from hydrogen or C₁-C₁₀ alkyl; or R₁₅ and R₁₆ taken together with the atom to which they are attached form a 4 to 10 membered cyclic ring with 1 or 2 nitrogen atoms and optionally an oxygen atom, said ring being substituted or unsubstituted;
X is N or N⁺O⁻
m is 0, 1, 2, 3,4 or 5; and
j is 1 or 2;
   wherein a total amount of the Compound A or a pharmaceutically acceptable salt thereof and the MEK inhibitor is therapeutically effective in treating the cancer.

Embodiment 3. A method of treating a subject suffering from cancer, comprising administering to the subject:
(i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof; and
   (ii) a MEK inhibitor or a pharmaceutically acceptable salt thereof selected from: cobimetinib, selumetinib, pimasertib, PD0325901, refametinib, binimetinib, BI-847325, trametinib, GDC-0623, G-573, and CH5126766,
      wherein a total amount of the Compound A or a pharmaceutically acceptable salt thereof and the MEK inhibitor or a pharmaceutically acceptable salt thereof is therapeutically effective in treating the cancer.

Embodiment 4. The method of any prior embodiment, wherein the cancer has one or more of the following mutations: RAS positive mutation, RAF positive mutation, MEK positive mutation, and ERK positive mutation.

Embodiment 5. The method of any prior embodiment 1 to 4, wherein the cancer has an NRAS mutation, a KRAS mutation, or HRAS mutation.

Embodiment 6. The method of any prior embodiment 1 to 4, wherein the cancer has a BRAF mutation, a BRAF fusion, or a CRAF fusion.

Embodiment 7. The method of embodiment 6, wherein the BRAF mutation is a non-V600 BRAF mutation.

Embodiment 8. The method of embodiment 7, wherein the non V600 BRAF mutation is selected from: V600E, G469A, G464V, G466V, K601E, G469R, and L597R.

Embodiment 9. The method of embodiment 8, wherein the non V600 BRAF mutation is selected from: V600E, G464A, G464V, K601E, and G469R.

Embodiment 10. The method of embodiment 9, wherein the non V600 BRAF mutation is selected from: G464V, K601E, G469A, and G466V.

Embodiment 11. The method of embodiment 5, wherein the RAS mutation is a KRAS mutation.

Embodiment 12. The method of embodiment 11, wherein the KRAS mutation is selected from: KRAS G12C, KRAS G12V, KRAS G12D, KRAS Q61K, KRAS Q61H, KRAS G13D, and KRAS G12S.

Embodiment 13. The method of any prior embodiment 11 or 12, wherein the KRAS mutation is selected from: KRAS G12C, KRAS G12D, KRAS G13D, and KRAS G12S.

Embodiment 14. The method of embodiment 6, wherein the BRAF mutation is a V600 BRAF mutation.

Embodiment 15. The method of any prior embodiment 1 to 8, wherein the cancer has a genomic alteration resulting in a dependency on signaling through the MAPK pathway.

Embodiment 16. The method of any prior embodiment 1 to 3, wherein the cancer has a mutation in NF-1 resulting in NF-1 loss-of function.

Embodiment 17. The method of any prior embodiment 1 to 3, wherein a cancer sample is taken from the subject.

Embodiment 18. The method of embodiment 17, wherein the cancer sample of the subject has been subjected to BRAF, KRAS, CRAF, HRAS, NF-1 and/or NRAS mutational testing prior to the administering of Compound A or a pharmaceutically acceptable salt thereof or the MEK inhibitor.

Embodiment 19. The method of embodiment 18, wherein the cancer sample of the subject has been subjected to genomic testing prior to the administering of Compound A or a pharmaceutically acceptable salt thereof or the MEK inhibitor, wherein the genomic testing demonstrates that genomic alteration creates a dependence on MPAK signaling.

Embodiment 20. The method of any prior embodiment 17 to 19, wherein the subject is diagnosed with histologically confirmed non-hematologic tumor.

Embodiment 21. The method of any prior embodiment 17 to 19, wherein the subject is diagnosed with histologically confirmed hematologic tumor.

Embodiment 22. The method of any prior embodiment 18 to 21, wherein the cancer has a RAS mutation.

Embodiment 23. The method of any prior embodiment 18 to 22, wherein the RAS mutation is an HRAS mutation, a KRAS, or an NRAS mutation.

Embodiment 24. The method of any prior embodiment 18 to 21, wherein the cancer has a mutation in NF-1 resulting in NF-1 loss-of function.

Embodiment 25. The method of any prior embodiment 18 to 21, wherein the cancer has a RAF alteration.

Embodiment 26. The method of embodiment 25, wherein the RAF alteration is a BRAF mutation, a BRAF fusion, or a CRAF fusion.

Embodiment 27. The method of embodiment 26, wherein the cancer has a non-V600 BRAF mutation.

Embodiment 28. The method of embodiment 27, wherein the subject is identified having a non V600 BRAF mutation selected from: V600E, G469A, G464V, G466V, K601E, G469R, and L597R.

Embodiment 29. The method of embodiment 28, wherein the subject is identified having a non V600 BRAF mutation selected from: V600E, G464A, G464V, K601E, and G469R.

Embodiment 30. The method of embodiment 29, wherein the subject is identified having a non V600 BRAF mutation selected from: G464V, K601E, G469A, and G466V.

Embodiment 31. The method of embodiment 23, wherein the subject is identified having a KRAS mutation.

Embodiment 32. The method of embodiment 31, wherein the subject is identified having a KRAS mutation selected from: KRAS G12C, KRAS G12V, KRAS G12D, KRAS Q61K, KRAS Q61H, KRAS G13D, and KRAS G12S.

Embodiment 33. The method of any prior embodiment 31 or 32, wherein the subject is identified having a KRAS mutation selected from: KRAS G12C, KRAS G12D, KRAS G13D, and KRAS G12S.

Embodiment 34. The method of any prior embodiment 1 to 21, wherein the subject has a Class I BRAF mutation or a Class II BRAF mutation.

Embodiment 35. The method of any prior embodiment 1 to 21, wherein the subject lacks V600E mutation, V600K mutation, or both.

Embodiment 36. The method of any prior embodiment 6 or 26, wherein the subject is identified having one or more of the following fusions: KIAA1549:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, AGK:BRAF, AGAP3:BRAF, TNS3:BRAF, TARDBP:BRAF, ARMC10:BRAF, CUL1:BRAF, TRIM24:BRAF, AKAP9:BRAF, FKBP15:BRAF, SKAP2:BRAF, ZKSCAN1:BRAF, KLHL7:BRAF, SEPT3:BRAF, SRGAP3:RAF1, QK1:RAF1, FYCO:RAF1, ATG7:RAF1, or NFIA:RAF1.

Embodiment 37. The method of embodiment 36, wherein the subject is identified having one or more of the following fusions: AGK:BRAF, AGAP3:BRAF, TNS3:BRAF, or KIAA1549:BRAF.

Embodiment 38. The method of any prior embodiment 36 or 37, wherein the subject is identified having KIAA1549:BRAF fusion.

Embodiment 39. The method of any prior embodiment 36 or 37, wherein the subject is identified as having AGAP3:BRAF fusion

Embodiment 40. The method of any prior embodiment 1 to 39, wherein the cancer is a solid tumor.

Embodiment 41. The method of embodiment 40, wherein the cancer is an advanced solid tumor.

Embodiment 42. The method of any prior embodiment 1 to 41, wherein the cancer is selected from lung cancer, colorectal cancer, pancreatic cancer, skin cancer, glioma, nonglioma brain cancer, bone sarcomas, gastrointestinal cancer, breast cancer, thyroid cancer, acute lymphocytic leukemia (ALL), acute myeloid leukemia (AML), and multiple myeloma (MM).

Embodiment 43. The method any prior embodiment 1 to 41, wherein the cancer is a lung cancer, colorectal cancer or pancreatic cancer.

Embodiment 44. The method of any prior embodiment 1 to 41, wherein the cancer is a non-small cell lung cancer.

Embodiment 45. The method of any prior embodiment 1 to 44, wherein the cancer is a recurrent cancer.

Embodiment 46. The method of any prior embodiment 1 to 45, wherein the subject has received at least one prior therapy that is considered standard of care treatment prior to the administration of Compound A or a pharmaceutically acceptable salt thereof, or the MEK inhibitor.

Embodiment 47. The method of embodiment 46, wherein the prior therapy is a systemic therapy.

Embodiment 48. The method of embodiment 46, wherein the prior therapy is chemotherapy therapy, hormone therapy, immunotherapy, or radiation therapy.

Embodiment 49 The method of any prior embodiment 1 to 48, wherein the MEK inhibitor is N-((S)-2,3-Dihydroxy-propyl)-3-(2-fluoro-4-iodo-phenylamino)-isonicotinamide (pimasertib) or a pharmaceutically acceptable salt thereof.

Embodiment 50. The method of any prior embodiment 1 to 48, wherein the MEK inhibitor is a compound having a structure of Formula (Ia) or a pharmaceutically acceptable salt thereof,

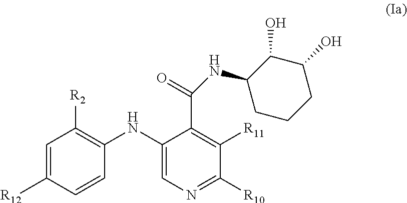

wherein,
R₂, R₁₂, R₁₀, and R₁₁ are independently selected from: hydrogen, halogen, cyano, nitro, azido, —OR₃, —NR₄C(O)OR₆, —OC(O)R₃, —NR₄S(O)ⱼR₆, —S(O)ⱼNR₃R₄, —S(O)ⱼNR₄C(O)R₃, —C(O)NR₄S(O)ⱼR₆, —S(O)ⱼR₆, —NR₄C(O)R₃, —C(O)NR₃R₄, —NR₅C(O)NR₃R₄, —NR₅C(NCN)NR₃R₄, —NR₃R₄, C₁-C₁₀ alkyl, C₂-C₁₀ alkenyl, C₂-C₁₀ alkynyl, C₃-C₁₀ cycloalkyl, C₃-C₁₀ cycloalkylalkyl, —S(O)ⱼ(C₁-C₆ alkyl), —S(O)ⱼ(CR₄R₅)ₘ-aryl, aryl, arylalkyl, heteroaryl, heteroarylalkyl, heterocyclyl, heterocyclylalkyl, —O(CR₄R₅)ₘ-aryl, —NR₄(CR₄R₅)ₘ-aryl, —O(CR₄R₅)ₘ-heteroaryl, —NR₄(CR₄R₅)ₘ, heteroaryl, —O(CR₄R₅)ₘ-heterocyclyl, —NR₄(CR₄R₅)ₘ-heterocyclyl and —S(C₁-C₂ alkyl) substituted with 1 to 5 fluorines, wherein each alkyl, alkenyl, alkynyl, cycloalkyl, aryl, heteroaryl and heterocyclyl is independently substituted or unsubstituted;
R₃ is selected from: hydrogen, trifluoromethyl, C₁-C₁₀ alkyl, C₂-C₁₀ alkenyl, C₂-C₁₀ alkynyl, C₃-C₁₀ cycloalkyl, C₃-C₁₀ cycloalkylalkyl, arylalkyl, heteroaryl, heteroarylalkyl, heterocyclyl, heterocyclylalkyl, and aryl; wherein each alkyl, alkenyl, alkynyl, cycloalkyl, heteroaryl and heterocyclyl is independently substituted or unsubstituted; and wherein aryl is optionally substituted with 1 to 5 groups independently selected from: oxo, halogen, nitro, CF₃, CHF₂, CH₂F, OCF₃, OCHF₂, OCH₂F, azido, NR′SO₂R″″, SO₂N″, C(O)R′, C(O)OR′, OC(0)R′, NR′C(O)OR″″, NR′C(O)R″, C(O)NR′R″, SR″″, S(0)R″″, SO₂R′, NR′R″, NR′C(O)NR″R′″, NR′C(NCN)N″R″′, OR′, aryl, heteroaryl, arylalkyl, heteroarylalkyl, heterocyclyl, and heterocyclylalkyl;
R₄ is selected from hydrogen or C₁₋₆ alkyl, wherein alkyl may be substituted or unsubstituted; or
R₃ and R₄ can be taken together with the atom to which they are attached to form a 4 to 10 membered heteroaryl or heterocyclic ring, each of which is substituted or unsubstituted;
R₅ is hydrogen or C₁-C₆ alkyl, wherein alkyl may be substituted or unsubstituted; or
R₄ and R₅ can be taken together with the atom to which they are attached to form a 4 to 10 membered carbocyclic, heteroaryl or heterocyclic ring, each of which is substituted or unsubstituted;
R₆ is selected from: trifluoromethyl, C₁-C₁₀ alkyl, C₃-C₁₀ cycloalkyl, aryl, arylalkyl, heteroaryl, heteroarylalkyl, heterocyclyl, and heterocyclylalkyl, wherein each alkyl, cycloalkyl, aryl, heteroaryl and heterocyclyl is independently substituted or unsubstituted;
R″, R″ and R″′ are independently selected from: hydrogen, C₁-C₄ alkyl, C₂-C₄ alkenyl, aryl and arylalkyl;
R″″ is selected from C₁-C₄ alkyl, C₂-C₄ alkenyl, aryl and arylalkyl;
m is 0, 1, 2, 3,4 or 5; and
j is 1 or 2.

Embodiment 51. The method of embodiment 50, wherein the MEK inhibitor is

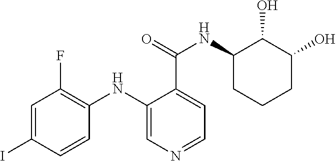 [SMILES: O=C(N[C@@H]1CCC[C@@H](O)[C@H]1O)c1ccncc1Nc1ccc(I)cc1F]

or a pharmaceutically acceptable salt thereof.

Embodiment 52. The method of any prior embodiment 1 to 48, wherein the MEK inhibitor or a pharmaceutically acceptable salt thereof is selected from:

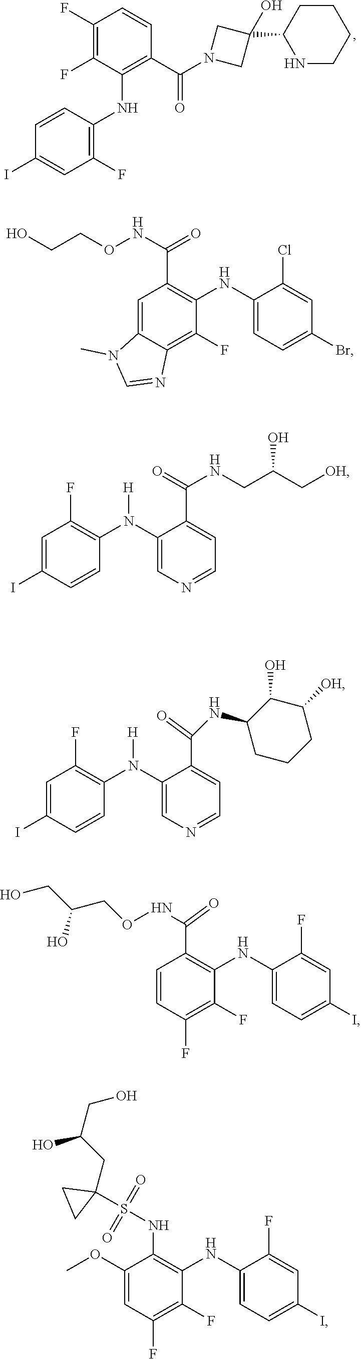 [SMILES: O=C(c1ccc(F)c(F)c1Nc1ccc(I)cc1F)N1CC(O)([C@@H]2CCCCN2)C1 ; Cn1cnc2c(F)c(Nc3ccc(Br)cc3Cl)c(C(=O)NOCCO)cc21 ; O=C(NC[C@H](O)CO)c1ccncc1Nc1ccc(I)cc1F ; O=C(N[C@@H]1CCC[C@@H](O)[C@H]1O)c1ccncc1Nc1ccc(I)cc1F ; O=C(NOC[C@H](O)CO)c1ccc(F)c(F)c1Nc1ccc(I)cc1F ; COc1cc(F)c(F)c(Nc2ccc(I)cc2F)c1NS(=O)(=O)C1(C[C@@H](O)CO)CC1]

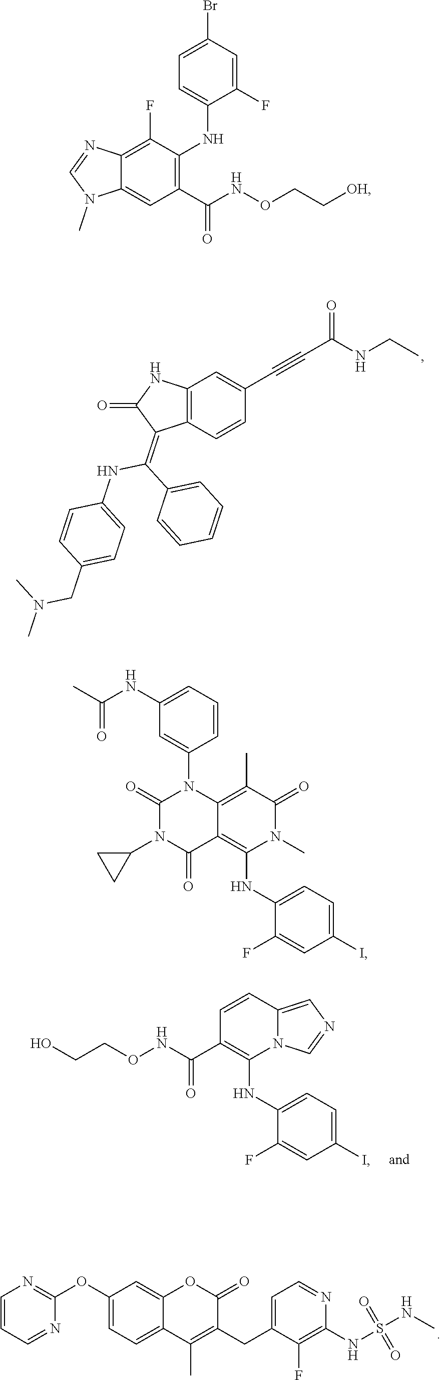

Embodiment 53. The method of any prior embodiment 1 to 48 or 52, wherein the MEK inhibitor or a pharmaceutically acceptable salt thereof is selected from:

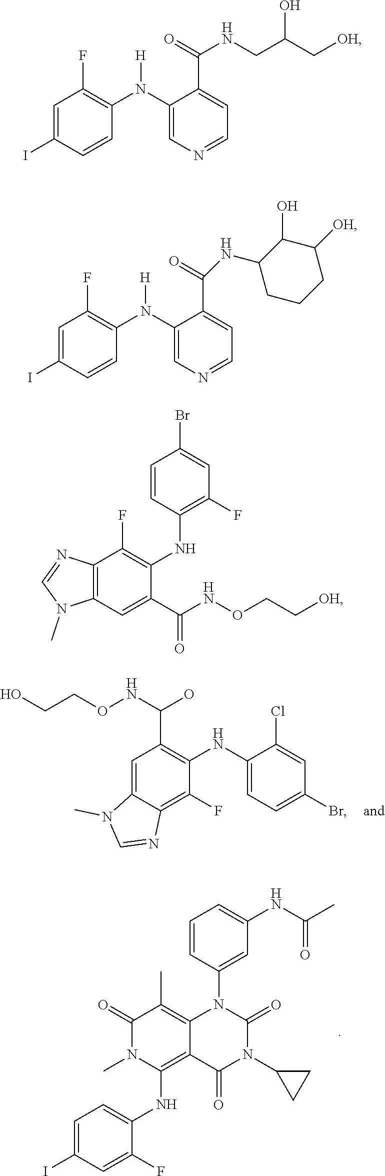

Embodiment 54. The method of any prior embodiment 1 to 53, wherein Compound A is administered in an amount of between about 100 mg to about 700 mg per week.

Embodiment 55. The method of embodiment 54, wherein Compound A is administered at about 200 mg, about 400 mg, or 600 mg per week.

Embodiment 56. The method of any prior embodiment 1 to 53, wherein Compound A is administered in an amount between about 100 mg/m² to about 500 mg/m² per week.

Embodiment 57. The method of embodiment 56, Compound A is administered at about 140 mg/m², about 280 mg/m², or about 420 mg/m² per week.

Embodiment 58. The method of any prior embodiment 1 to 57, wherein Compound A is administered once weekly.

Embodiment 59. The method of any prior embodiment 1 to 58, wherein the MEK inhibitor is administered in an amount between about 10 mg to about 150 mg daily.

Embodiment 60. The method of any prior embodiment 1 to 58, wherein the MEK inhibitor is administered in an amount between about 5 mg to about 75 mg twice daily.

Embodiment 61. The method of embodiment 60, wherein the MEK inhibitor is administered at about 15 mg, about 30 mg, about 45 mg, or about 60 mg twice daily.

Embodiment 62. The method of any prior embodiment 1 to 61, wherein the subject has not been previously administered a pan-RAF therapy.

Embodiment 63. The method of any prior embodiment 1 to 62, wherein the subject has not been previously administered a cytochrome P450 CYP3A4 inhibitor, a cytochrome P450 CYP2C19 inhibitor, a P450 CYP3A4 inducer, or a substrate of CYP2C9.

1. A method of treating a subject suffering from cancer, comprising administering to the subject:
(i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof; and
(ii) a MEK inhibitor or a pharmaceutically acceptable salt thereof, wherein the MEK inhibitor is pimasertib,
wherein Compound A or a pharmaceutically acceptable salt thereof and pimasertib or a pharmaceutically acceptable salt thereof are administered in a therapeutically effective amount for treating the cancer.

2. The method of claim 1, wherein the Compound A or a pharmaceutically acceptable salt thereof is administered to the subject in an amount of about 50 mg to about 800 mg per week or in an amount of about 100 mg/m² to about 600 mg/m² per week, and wherein the pimasertib or a pharmaceutically acceptable salt thereof is administered to the subject in an amount of about 5 mg to about 150 mg daily.

3. The method of claim 2, wherein the Compound A or a pharmaceutically acceptable salt thereof is administered to the subject in an amount of about 200 mg to about 600 mg per week or in an amount of about 140 mg/m² to about 420 mg/m² per week, and wherein the pimasertib or a pharmaceutically acceptable salt thereof is administered to the subject in an amount of about 10 mg to about 60 mg daily.

4. The method of claim 1, comprising administering Compound A, and a HCl salt of pimasertib.

5. (canceled)

6. The method of claim 1, wherein the subject is identified as having one or more of the following fusions: AGK:BRAF, BRAF-AGAP3, AGAP3:BRAF, TNS3:BRAF, or KIAA1549:BRAF.

7. (canceled)

8. The method of claim 1, wherein the subject is identified as having a mutation selected from: KRAS G12C, KRAS G12D, and KRAS G12S.

9. (canceled)

10. The method of claim 1, wherein the subject is identified as having a BRAF mutation selected from: BRAF G464V, BRAF Indel, BRAF L597R, BRAF G466V, BRAF G469A, BRAF K601E, and BRAF G469R.

11. (canceled)

12. The method of claim 1, wherein the cancer is a recurrent, progressive, or refractory solid tumor with mitogen-activated protein kinase (MAPK) pathway aberration.

13. A method of treating a subject suffering from cancer, comprising administering to the subject:
(i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof; and
(ii) a MEK inhibitor or a pharmaceutically acceptable salt thereof,
wherein Compound A or a pharmaceutically acceptable salt thereof and the MEK inhibitor or a pharmaceutically acceptable salt thereof are administered in a therapeutically effective amount for treating the cancer, and
wherein the Compound A or a pharmaceutically acceptable salt thereof is administered to the subject in an amount of about 50 mg to about 800 mg per week or in an amount of about 100 mg/m² to about 600 mg/m² per week.

14. A method of treating a subject suffering from cancer, comprising administering to the subject:
(i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof; and
(ii) a MEK inhibitor or a pharmaceutically acceptable salt thereof,
wherein Compound A or a pharmaceutically acceptable salt thereof and the MEK inhibitor or a pharmaceutically acceptable salt thereof are administered in a therapeutically effective amount for treating the cancer, and
wherein the subject has one or more mitogen-activated protein kinase (MAPK) pathway aberration.

15. The method of claim 14, wherein the MAPK pathway aberration is selected from one or more BRAF mutations or fusions and KRAS mutations or fusions.

16. The method of claim 15, wherein the BRAF mutations or fusions and KRAS mutations for fusions is selected from the following gene mutations or gene fusions: BRAF V600E, BRAF G464V, BRAF G466V, BRAF G464V, BRAF K601E, KRAS Q61, KRAS G12S, BRAF G464V, BRAF Indel, BRAF L597R, BRAF G466V, BRAF G469A, BRAF K601E, BRAF G469R, KRAS G12C, KRAS G12D, KRAS G12S, AGK:BRAF, BRAF-AGAP3, AGAP3:BRAF, TNS3:BRAF, or KIAA1549:BRAF.

17. (canceled)

18. (canceled)

19. The method of claim 14, wherein the MEK inhibitor is pimasertib.

20-22. (canceled)

23. The method of claim 1, wherein the cancer has a BRAF mutation, a BRAF fusion, or a CRAF fusion.

24-31. (canceled)

32. The method of claim 1, wherein the subject is identified having one or more of the following fusions: KIAA1549:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, AGK:BRAF, AGAP3:BRAF, TNS3:BRAF, TARDBP:BRAF, ARMC10:BRAF, CUL1:BRAF, TRIM24:BRAF, AKAP9:BRAF, FKBP15:BRAF, SKAP2:BRAF, ZKSCAN1:BRAF, KLHL7:BRAF, SEPT3:BRAF, SRGAP3:RAF1, QK1:RAF1, FYCO:RAF1, ATG7:RAF1, or NFIA:RAF1.

33-35. (canceled)

36. The method of claim 23, wherein the BRAF mutation is a non-V600 BRAF mutation and wherein the non-V600 BRAF mutation is selected from: V600E, G469A, G464V, G466V, K601E, G469R, and L597R.

37-41. (canceled)

42. The method of claim 14, wherein Compound A is administered in an amount of about 100 mg to about 700 mg per week, wherein the subject is at least 18 years of age.

43. (canceled)

44. (canceled)

45. The method of claim 14, wherein Compound A is administered in an amount between about 100 mg/m² to about 500 mg/m² per week, wherein the subject is 12, 13, 14, 15, 16, or 17 years of age.

46-48. (canceled)

49. The method of claim 14, wherein the MEK inhibitor or a pharmaceutically acceptable salt thereof is administered in an amount of about 10 mg to about 150 mg daily.

50-52. (canceled)

53. The method of claim 1, wherein the subject has not been previously administered a cytochrome P450 CYP3A4 inhibitor, a cytochrome P450 CYP2C19 inhibitor, a P450 CYP3A4 inducer, or a substrate of CYP2C9.

54. The method of claim 1, wherein the cancer is a solid tumor.

55. (canceled)

56. The method of claim 1, wherein the cancer is selected from lung cancer, colorectal cancer, pancreatic cancer, skin cancer, glioma, nonglioma brain cancer, bone sarcomas, gastrointestinal cancer, breast cancer, thyroid cancer, acute lymphocytic leukemia (ALL), acute myeloid leukemia (AML), and multiple myeloma (MM).

57. The method of claim 56, wherein the cancer is melanoma.

58-59. (canceled)

60. The method of claim 1, wherein the subject has received at least one prior therapy that is considered standard of care treatment prior to the administration of Compound A or a pharmaceutically acceptable salt thereof, or the MEK inhibitor.

61-65. (canceled)

66. The method of claim 1, wherein the subject is identified as having a BRAF fusion that is TRIM:BRAF or AGK:BRAF.
